SEQUENCE LISTING (56 sequences)

SEQ ID NO: 1 - DNA, 5400 bp, Homo sapiens
ccgccaagcatattgctaggcacagagcaggtgtgcaacaaaagttatttctcaggctttccctcctctgagcgccgtcctccagagggtccggagtgtagctgggggttggagcagcagcctcctaggcgatgggacagagcccacagggtccggtatgccacggtttcttcgtcagaccctgggaatccaacgtcgcaaaataaacacggccgcgccgctaatcgccagttcggaggaaacaaaacagcgctgcgctgggggatctgggcaaaatcagccctccctcctcccgctccttcgccgcggccctcccctcctcgcgctgctctcgttcgcttggctcagctcagctcagctcagcgcagctccgcggccgccaagccgaggcgggcacggtctccgagtcgcggacgccagctccgagctccctctctccgccgcgcctccgccaggtcgcgccttcgtcgggaccacttcgggcaggagtcgcgtggcgaaggcctgcggccgcggcacaaagttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccaatggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggctccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtgcagcgccacctgcggcatgggcatgaagaagcggcaccgcatgatcaagatgaaccccgcagatggctccatgtgcaaagccgagacatcacaggcagagaagtgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagctacgctggagggaggcccgagagagccggcggagtgagcagctgaaggaagagtctgaaggggagcagttcccaggttgtaggatgcgcccatggacggcctggtcagaatgcaccaaactgtgcggaggtggaattcaggaacgttacatgactgtaaagaagagattcaaaagctcccagtttaccagctgcaaagacaagaaggagatcagagcatgcaatgttcatccttgttagcaagggtacgagttccccagggctgcactctagattccagagtcaccaatggctggattatttgcttgtttaagacaatttaaattgtgtacgctagttttcatttttgcagtgtggttcgcccagtagtcttgtggatgccagagacatcctttctgaatacttcttgatgggtacaggctgagtggggcgccctcacctccagccagcctcttcctgcagaggagtagtgtcagccaccttgtactaagctgaaacatgtccctctggagcttccacctggccagggaggacggagactttgacctactccacatggagaggcaaccatgtctggaagtgactatgcctgagtcccagggtgcggcaggtaggaaacattcacagatgaagacagcagattccccacattctcatctttggcctgttcaatgaaaccattgtttgcccatctcttcttagtggaactttaggtctcttttcaagtctcctcagtcatcaatagttcctggggaaaaacagagctggtagacttgaagaggagcattgatgttgggtggcttttgttctttcactgagaaattcggaatacatttgtctcacccctgatattggttcctgatgcccccccaacaaaaataaataaataaattatggctgctttatttaaatataaggtagctagtttttacacctgagataaataataagcttagagtgtatttttcccttgcttttgggggttcagaggagtatgtacaattcttctgggaagccagccttctgaactttttggtactaaatccttattggaaccaagacaaaggaagcaaaattggtctctttagagaccaatttgcctaaattttaaaatcttcctacacacatctagacgttcaagtttgcaaatcagtttttagcaagaaaacatttttgctatacaaacattttgctaagtctgcccaaagcccccccaatgcattccttcaacaaaatacaatctctgtactttaaagttattttagtcatgaaattttatatgcagagagaaaaagttaccgagacagaaaacaaatctaagggaaaggaatattatgggattaagctgagcaagcaattctggtggaaagtcaaacctgtcagtgctccacaccagggctgtggtcctcccagacatgcataggaatggccacaggtttacactgccttcccagcaattataagcacaccagattcagggagactgaccaccaagggatagtgtaaaaggacattttctcagttgggtccatcagcagtttttcttcctgcatttattgttgaaaactattgtttcatttcttcttttataggccttattactgcttaatccaaatgtgtaccattggtgagacacatacaatgctctgaatacactacgaatttgtattaaacacatcagaatatttccaaatacaacatagtatagtcctgaatatgtacttttaacacaagagagactattcaataaaaactcactgggtctttcatgtctttaagctaagtaagtgttcagaaggttcttttttatattgtcctccacctccatcattttcaataaaagatagggcttttgctcccttgttcttggagggaccattattacatctctgaactacctttgtatccaacatgttttaaatccttaaatgaattgctttctcccaaaaaaagcacaatataaagaaacacaagatttaattatttttctacttggggggaaaaaagtcctcatgtagaagcacccacttttgcaatgttgttctaagctatctatctaactctcagcccatgataaagttccttaagctggtgattcctaatcaaggacaagccaccctagtgtctcatgtttgtatttggtcccagttgggtacattttaaaatcctgattttggagacttaaaaccaggttaatggctaagaatgggtaacatgactcttgttggattgttattttttgtttgcaatggggaatttataagaagcatcaagtctctttcttaccaaagtcttgttaggtggtttatagttcttttggctaacaaatcattttggaaataaagattttttactacaaaaatgaaatttgtttggacttccacttgagacagtaaagagagtattagacacccagtaaaaactgccatataaagaagttgtaattgtttgttgtgtatgtatttttttcaatgccaaaccagctgtgatccaatttacatccacattttaggtccaacagcaagaagttcagagagagatttcccaaccagacattgggtcactcactggtcaccttgccagtgcattttattagaagggaatctgttgtagcaaatgggaataaacctgggtttctatagacccagaactgaaaaaataaaaaaaaaaaaaaaaaa

SEQ ID NO: 2 - protein, 807 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGSTCTMSEWITWSPCSISCGMGMRSRERYVKQFPEDGSVCTLPTEETEKCTVNEECSPSSCLMTEWGEWDECSATCGMGMKKRHRMIKMNPADGSMCKAETSQAEKCMMPECHTIPCLLSPWSEWSDCSVTCGKGMRTRQRMLKSLAELGDCNEDLEQVEKCMLPECPIDCELTEWSQWSECNKSCGKGHVIRTRMIQMEPQFGGAPCPETVQRKKCRIRKCLRNPSIQKLRWREARESRRSEQLKEESEGEQFPGCRMRPWTAWSECTKLCGGGIQERYMTVKKRFKSSQFTSCKDKKEIRACNVHPC

SEQ ID NO: 3 - DNA, 4029 bp, Rattus norvegicus
ccctccctcttcgcgctccttcgccaccgcccgcccctcagctccgctgctcggctccgctcagagcagcgcagctccgcagccaaagcgaggcgggctcgggctccccaccgccagtgccacccgggctcctccagctttcgcctctgcagctcccgtcacttggagtaaaagtgtcctgacaggggtctgcaacatcagcagaaagttgggaggtcctcgagaatgaggctatctcccgcgcccctgaggcttagccggggtccggcgctgctggccctggcgctgcccctggccgcagcgctcgctttctcggatgagaccctggacaaagtggccaagtcggagggctactgcagccgcatcttgcgcgcccagggcacacggcgtgagggatacacagagttcagcctccgcgtggaaggcgaccctgacttctataagccaggaagcagctaccgagtgacactctcggctgcccctccctcctacttcagaggcttcacgttaattgctctcaaagagaaccgcgaaggcgataaggaagaagaccacgcgggcaccttccagatcatagatgaagaagaaacccagtttatgagtaactgtcctgtggcagtcactgaaagcacccctcggaggaggacacggatccaggtgttttggatagcgccacccacagggacaggctgtgtgattctgaaggccagcattgtacagaaacgcattatctattttcaagacgagggctccctgaccaagaagctgtgtgaacaggatcccacacttgatggagtgacggacagaccgatcttagactgctgcgcctgcggaactgccaagtacagactcacgttttatgggaactggtcggagaagactcatccaaaggattaccctcgtcgggctaatcactggtctgccatcattggcggatcccactccaagaactacgtgctgtgggagtacggagggtatgccagtgaaggggtcaagcaagttgctgaacttggctcaccagtaaaaatggaggaagaaattcgacaacagagtgatgaagtcctcactgtcatcaaagccaaagcccagtggccatcctggcagcctgtcaatgtgagagcagcaccctcagccgaattctcagtggacaggacacgccacttgatgtccttcctaaccatgatgggccccagtcctgactggaacgtgggcctatctgcagaggatctgtgcaccaaggagtgtggctgggtccagaaagtggtgcaggacctaattccctgggatgctggcacggacagcggggtgacctacgagtcaccaaacaagcccacaattcctcaggaaaaaatccgacccctgactagtctggaccatcctcagagtcctttctatgacccggaaggtgggtccatcacacaagtggccagagtcgtcatcgagagaattgcccggaagggagaacaatgcaacattgtacctgacaatgtggatgatattgtagccgacctggctccagaagagaaagatgaagatgacacccctgaaacctgcatctactccaactggtccccatggtcggcctgcagctcttccacttgtgaaaagggtaagaggatgcggcaacgcatgctgaaggcacagctggacctcagtgtcccctgtcctgacacccaggacttccagccctgcatgggccccggctgcagcgatgaagatggctccacctgtaccatgtcggagtggatcacctggtcaccctgcagtgtctcgtgtggcatgggtatgaggtcccgggagaggtacgtgaagcagttcccggaagacggctcggtgtgcatgctgcccacggaagagacagagaagtgcacggtcaacgaggagtgctctcctagcagctgcctggtgactgagtggggtgagtgggatgactgcagcgccacctgtggaatgggcatgaagaagcggcaccgcatggtcaagatgagccccgcggacggctccatgtgcaaggcggagacttcgcaggcggagaaatgcatgatgcctgagtgccataccatcccgtgcttgctgtctccttggtccgagtggagcgactgtagcgtgacctgtgggaagggcatgcggacgcgccagcggatgctcaagtctctggcagagctgggggactgtaatgaggatctggagcaggcggagaagtgtatgctgccagagtgccccattgactgcgaactcagtgagtggtcccagtggtctgaatgtaacaagtcctgtgggaaaggtcacatgattcgaacccggacaatccaaatggaacctcagtttggaggtgcaccctgcccagagactgtgcaacgcaagaagtgccgtgcccggaaatgccttcgcagcccatcgatccagaagctgcgctggagggaggcccgagagagcaggaggagtgagcagctgagggaagagtcagatggagagcagttcccaggctgtcggatgcgcccgtggacagcctggtcagagtgcaccaaactgtgcggaggtgggatccaagaacgctacatgactgtgaagaagaggttcaaaagctcccagtttaccagctgcaaagacaagaaggagatcagagcgtgcaacgtgcacccttgttagtaggggttcaactccccagggctgcattccagattctagtcaccaatggttgggtggtgtatttgcttgtttaagatgatttaaattgtgtccacatgttttcatttttaccggtgtggtttgcccaatagtcttatggaggccgagggacatcttgtctgaatacttcttggtgagtacaggccaagcggggcatcttgtccccaggcgccatcttcctgcactgagttgagtagtgttggttcaccttggtactaaactgaatcgtgtccctctggagcatcccctggtcaagcagggtggagactttggccatccacaaggagaagcaaccaggatgcagcatgcgggagacacagccattaattgcaaaggacagatcctcctctctcacctttggcctgctcactcttacagaaacctgtttgtccgcctccttttttatttagcacaactccaggcatcttggtaagtctccagggtcatgggttcttcggtgccctgaaggagaagccctgaggtgaggtggcatttgttacaaacctcccaatactgctttactggcatcacaaggtcagcaggtgatgatggctacttcatttcattgtgagccgtgatttccgttgagttttgattgttggtgccataaatgtcctaggatgctggacggacacatcagccttgtcagcagatccttctttgagccaatgtagacagtaagctgggcactggttccaaagccaacttaaaatcttcctacacatatccagacctttttttaggttgcccaaacttccttagaataaagcattttagctctgagaactacttgataagtctgccaggaagcccccaagtcaattcttcaacaaaaatactatcttccctacttaattttttttaagtcatgatattttatagttagaggagagagagacaatctattcccatgactaagacacaaacctacaagaaagggttactcagtcaagcctgtgcctgacttctggaccaggcccctgattttcatggatagtccaaaggaaggccaggggttcccactgactccaagccatcagcagcacccaaacccaggagcaacaaatattcagagaaagaggatgtttatctcagctatgagctcattggcaggttgtactcatgcatctgttaaaagcaccaccacatccttttgcaagtctgtttattaccgcttcatccaaatacattttgtggtcaagatcgacacagtgctatgaatacagtactttaaggtctgcattaaacacatcagaatatttcctgccacatctatgtacaacccctgaatatgtatttttccttaacacaagagagcctgttcaattaaaaaaaaaaaaa

SEQ ID NO: 4 - protein, 807 aa, Rattus norvegicus
MRLSPAPLRLSRGPALLALALPLAAALAFSDETLDKVAKSEGYCSRILRAQGTRREGYTEFSLRVEGDPDFYKPGSSYRVTLSAAPPSYFRGFTLIALKENREGDKEEDHAGTFQIIDEEETQFMSNCPVAVTESTPRRRTRIQVFWIAPPTGTGCVILKASIVQKRIIYFQDEGSLTKKLCEQDPTLDGVTDRPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPSWQPVNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCEKGKRMRQRMLKAQLDLSVPCPDTQDFQPCMGPGCSDEDGSTCTMSEWITWSPCSVSCGMGMRSRERYVKQFPEDGSVCMLPTEETEKCTVNEECSPSSCLVTEWGEWDDCSATCGMGMKKRHRMVKMSPADGSMCKAETSQAEKCMMPECHTIPCLLSPWSEWSDCSVTCGKGMRTRQRMLKSLAELGDCNEDLEQAEKCMLPECPIDCELSEWSQWSECNKSCGKGHMIRTRTIQMEPQFGGAPCPETVQRKKCRARKCLRSPSIQKLRWREARESRRSEQLREESDGEQFPGCRMRPWTAWSECTKLCGGGIQERYMTVKKRFKSSQFTSCKDKKEIRACNVHPC

SEQ ID NO: 5 - DNA, 4035 bp, Mus musculus
cggacgcgtgggcagctccgctgctcggctccgctcagagcagcgcagctccgcagccgccaaagcgaggcgggcacggtctccccaccgccagcgccacccgggctcctccaggtttcgcctctgcagctcccgtcacttggagcaggagtgtcctgacagggtctgcaacatccgcataaagttggggggccctcgaggatgaggctgtctcccgtgtccctgaggcttagccggggtccggcgctgctggccctcgcgctgcccctggccgcagcgctagctttctcagatgagaccctggacaaagtgaccaagtcggagggctactgcagccgcatcttgcgcgcccagggcacacggcgtgagggatacacggagttcagcctccgcgtggaaggcgaccctgacttctataagccaggaagcagctaccgagtgacactttcggctgctcctccctcctacttcagaggtttcacgttaattgccctcaaagagaaccaggagggagataaggaagaagatcatgcaggcaccttccagatcatagatgaggaagagacccagtttatgagtaactgtcctgtggccgtcactgaaagcacccctcggaggaggacacggatccaggtgttttggatagcgccaccaacagggacaggctgtgtgattctgaaggccagcattgtacagaaacgcataatttattttcaagacgagggctccctgaccaagaagctgtgtgaacaggatcccacacttgatggggtgacggacagacccatcttagactgctgcgcctgtggaactgccaagtacagactcacgttttatgggaactggtccgagaagactcatccgaaggattaccctcgtcgggctaaccactggtctgccatcattggtgggtcccactccaagaactacgtgctgtgggagtatggagggtatgccagcgaaggggtcaagcaagttgctgaactgggctcaccagtaaaaatggaggaagaaattcgacaacagagtgatgaagtcctcactgtcatcaaagccaaagcccagtggccagcctggcagcctgtcaatgtgagagcagcaccctcagctgaattctcagtggacaggacacgccacttgatgtccttcctcaccatgatgggccccagtccagactggaacgtgggcctatctgcagaggatctgtgcaccaaggagtgtggctgggtccagaaagtagtgcaggacctgattccctgggatgctggcactgacagcggcgtgacctatgagtcaccaaacaagcccacaattcctcaggaaaaaatccgacccctgactagtctggatcatcctcagagtcctttctatgacccagaaggtgggtccatcacacaagtggccagagtcgtcatcgagagaattgcccggaagggagaacagtgcaacattgtacctgacaacgtggacgatattgtagccgacctggctccagaagagaaagatgaagatgacacccctgaaacctgcatctactccaactggtccccatggtcggcctgcagctcttccacttgtgaaaagggcaagaggatgcggcagcgcatgctgaaggcacagctggacctcagtgtcccctgtcctgacacccaggacttccagccctgcatgggcccaggctgcagcgatgaagatggctccacctgtaccatgtcggagtggatcacctggtcgccctgtagcgtctcgtgcggcatgggcatgaggtcccgggagagatacgtgaagcagttcccggaagacggctcagtgtgcatgctgcccacggaggagacggagaagtgcacagtcaacgaggagtgctctcctagcagctgcctggtgactgagtggggtgagtgggacgactgcagtgccacctgtgggatgggtatgaagaagaggcaccgtatggtcaagatgagccccgcggacggctccatgtgcaaggcagagacatcgcaggcggagaaatgcatgatgcctgagtgccataccatcccgtgcttgctgtctccctggtccgagtggagcgactgtagcgtgacctgtgggaagggcatgcggacccgccagcggatgctcaaatctctggcagagctgggtgactgtaacgaggacctggagcaggcggagaagtgcatgctgccagaatgccccattgactgtgaactcagcgagtggtcccagtggtctgaatgtaacaagtcatgtgggaaaggtcacatgattcgaacccggacaatccaaatggaacctcagtttgggggtgtaccctgcccagagactgtgcaacgcaagaagtgccgcacccggaaatgccttcgcagcccatcagtccagaagctgcgctggagggaggcccgagagagcaggaggagtgagcagctgagagaagagtcagatggagagcagttcccaggctgtcgaatgcgcccgtggacagcctggtcagaatgcaccaaactgtgcggaggtgggatccaagagcgctacatgactgtgaagaagaggttcaaaagctcccagtttaccagctgcaaagacaagaaggagatcagagcgtgcaacgtgcacccttgttaatgggggttcaactccccacggccgcattccagattctagtcaccaatggttgggtggtttgtttgcttgtgtaagatgatttcaattgtgtccactagttttctttcttttttttttttattacgatgtggtttgcccaatagtcttatgatgccaagggacagcctgtccgaattcttcttggtgagtgcaggccaagtggggcatccttgtcctcaggtggccctcttcctacactgagtgagtagtgttggctcaccttgtgtccctctggaacatccccctggtcaagcagggtgaagaccctggccacatccacaaggaaaagcaaccatgttctcgggtgccagaatgcagcatgcaggagacacagccatcaaatgcagaggacagattctcctctctcatctgcggcctgctcactcttacagaaacccgttcacccatctctttttttatttagcacaactccaggtatcttggtaagtctccagggtcacgggttcttcagtgccctgaaggaggagctctaaggtaggggtggcgtttgttacaaacctcctgatactgctttgttgacaccacaagggtagcaggtgatgatggctactttatttgtgagccatgatttccgttgagttctgattgttggtgccataagtgtccgagggtgccggacggacgcactaaccttttctcggcagatccttctctgagccaatgcagacggtctactgggcactggttccaaagccaccctaaaatcttcctacacatatgcaaaccttttaggttccccaatcttccttagaataaaacattttagctctgagaactacctgataagtcggccagagagccaccaagtcaatcctcaacaaaaataccatcttccctacttaaaagttttttttaaagtcgtgatattttatagttagaggagagagagagagagacaaactattcccatgactaagatacaaacctacaagtaagggttattcagtcatgcctgtgcctgatttctggaccagggccctggttttcatggacagtccagaggaaagtcgggggtttccactgatttccaagctatcagcagcacactaaacccaggagtctaagaaccaacagtcaagagtaagaggatgttgatctcagctgtgagctcattagcaggttttactcatgcatctgttaaagctaccaccacatccttttgcaagtctgtttattaccgctttatccaaatacatactgtgggcaagatgcacacagtgctatgaatacagtactttaagaatctccattcaacatatcagaatatgtccagccacatctatgtacaacccctgaatatgtattttccttaacacacgagagcctgttcaattaaaaaaaaaaaaaaaa

SEQ ID NO: 6 - protein, 807 aa, Mus musculus
MRLSPVSLRLSRGPALLALALPLAAALAFSDETLDKVTKSEGYCSRILRAQGTRREGYTEFSLRVEGDPDFYKPGSSYRVTLSAAPPSYFRGFTLIALKENQEGDKEEDHAGTFQIIDEEETQFMSNCPVAVTESTPRRRTRIQVFWIAPPTGTGCVILKASIVQKRIIYFQDEGSLTKKLCEQDPTLDGVTDRPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPVNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCEKGKRMRQRMLKAQLDLSVPCPDTQDFQPCMGPGCSDEDGSTCTMSEWITWSPCSVSCGMGMRSRERYVKQFPEDGSVCMLPTEETEKCTVNEECSPSSCLVTEWGEWDDCSATCGMGMKKRHRMVKMSPADGSMCKAETSQAEKCMMPECHTIPCLLSPWSEWSDCSVTCGKGMRTRQRMLKSLAELGDCNEDLEQAEKCMLPECPIDCELSEWSQWSECNKSCGKGHMIRTRTIQMEPQFGGVPCPETVQRKKCRTRKCLRSPSVQKLRWREARESRRSEQLREESDGEQFPGCRMRPWTAWSECTKLCGGGIQERYMTVKKRFKSSQFTSCKDKKEIRACNVHPC

SEQ ID NO: 7 - DNA, 3935 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctacatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaagaaa

SEQ ID NO: 8 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 9 - DNA, 5572 bp, Homo sapiens
gtgtgaatgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcagatgatgacactgccacctctgacttctgcctctggccttccactctcagtaagaagagccaggcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgttaaaaaaaaaaaaaaaaaaaa

SEQ ID NO: 10 - protein, 589 aa, Homo sapiens
MLHSPHKQPQNHKCGANFLQEDSKKSLVFKWLISAGHYQPPRPTESFKAASSIYNRGYKFYLKKKGGTMASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 11 - DNA, 4465 bp, Homo sapiens
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 12 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 13 - DNA, 2346 bp, Homo sapiens
ccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatc

SEQ ID NO: 14 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 15 - DNA, 5087 bp, Homo sapiens
aacgggcgccgcggcggggagaagacgcagagcgctgctgggctgccgggtctcccgcttccccctcctgctccaagggcctcctgcatgagggcgcggtagagacccggacccgcgccgtgctcctgccgtttcgctgcgctccgcccgggcccggctcagccaggccccgcggtgagccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagtaca

SEQ ID NO: 16 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 17 - DNA, 7137 bp, Homo sapiens
cggccaggtgtgtgggactgaagttcttggagaagggagtccaactcttcaaggtgaactatgaccactttactctgggttttcgtgactctgagggtcatcactgcagctgtcactgtagaaacttcagaccatgacaactcgctgagtgtcagcatcccccaaccgtccccgctgagggtcctcctggggacctccctcaccatcccctgctatttcatcgaccccatgcaccctgtgaccaccgccccttctaccgccccactggccccaagaatcaagtggagccgtgtgtccaaggagaaggaggtagtgctgctggtggccactgaagggcgcgtgcgggtcaacagtgcctatcaggacaaggtctcactgcccaactacccggccatccccagtgacgccaccttggaagtccagagcctgcgctccaatgactctggggtctaccgctgcgaggtgatgcatggcatcgaggacagcgaggccaccctggaagtcgtggtgaaaggcatcgtgttccattacagagccatctctacacgctacaccctcgactttgacagggcgcagcgggcctgcctgcagaacagtgccatcattgccacgcctgagcagctgcaggccgcctacgaagacggcttccaccagtgtgacgccggctggctggctgaccagactgtcagataccccatccacactccccgggaaggctgctatggagacaaggatgagtttcctggtgtgaggacgtatggcatccgagacaccaacgagacctatgatgtgtactgcttcgccgaggagatggagggtgaggtcttttatgcaacatctccagagaagttcaccttccaggaagcagccaatgagtgccggcggctgggtgcccggctggccaccacgggccacgtctacctggcctggcaggctggcatggacatgtgcagcgccggctggctggccgaccgcagcgtgcgctaccccatctccaaggcccggcccaactgcggtggcaacctcctgggcgtgaggaccgtctacgtgcatgccaaccagacgggctaccccgacccctcatcccgctacgacgccatctgctacacaggtgaagactttgtggacatcccagaaaacttctttggagtggggggtgaggaggacatcaccgtccagacagtgacctggcctgacatggagctgccactgcctcgaaacatcactgagggtgaagcccgaggcagcgtgatccttaccgtaaagcccatcttcgaggtctcccccagtcccctggaacccgaggagcccttcacgtttgcccctgaaataggggccactgccttcgctgaggttgagaatgagactggagaggccaccaggccctggggctttcccacacctggcctgggccctgccacggcattcaccagtgaggacctcgtcgtgcaggtgaccgctgtccctgggcagccgcatttgccagggggggtcgtcttccactaccgcccgggacccacccgctactcgctgacctttgaggaggcacagcaggcctgccctggcacgggggcggtcattgcctcgccggagcagctccaggccgcctacgaagcaggctatgagcagtgtgacgccggctggctgcgggaccagaccgtcagataccccattgtgagcccacggaccccatgcgtgggtgacaaggacagcagcccaggggtcaggacctatggcgtgcgcccatcaacagagacctacgatgtctactgctttgtagacagacttgagggggaggtgttcttcgccacacgccttgagcagttcaccttccaggaagcactggagttctgtgaatctcacaatgccactgccaccacgggccagctctacgccgcctggagccgcggcctggacaagtgctatgccggctggctggccgacggcagcctccgctaccccatcgtcaccccaaggcctgcctgcggtggggacaagccaggcgtgagaacggtctacctctaccctaaccagacgggcctcccagacccactgtcccggcaccatgccttctgcttccgaggcatttcagcggttccttctccaggagaagaagagggtggcacacccacatcaccctctggtgtggaggagtggatcgtgacccaagtggttcctggtgtggctgctgtccccgtagaagaggagacaactgctgtaccctcaggggagactactgccatcctagagttcaccaccgagccagaaaaccagacagaatgggaaccagcctataccccagtgggcacatccccgctgccagggatccttcctacttggcctcctactggcgccgaaacagaggaaagtacagaaggcccttctgcaactgaagtgccctctgcctcagaggaaccatccccctcagaggtgccattcccctcagaggagccatccccctcagaggaaccattcccctcagtgaggccattcccctcagtggagctgttcccctcagaggagccattcccctccaaggagccatccccctcagaggaaccatcagcctcagaagagccgtatacaccttcaccccccgagcccagctggactgagctgcccagctctggggaggaatctggggcccctgatgtcagtggtgacttcacaggcagtggagatgtttcaggacaccttgacttcagtgggcagctgtcaggggacagggcaagtggactgccctctggagacctggactccagtggtcttacttccacagtgggctcaggcctgactgtggaaagtggactaccctcaggggatgaagagagaattgagtggcccagcactcctacggttggtgaactgccctctggagctgagatcctagagggctctgcctctggagttggggatctcagtggacttccttctggagaagttctagagacctctgcctctggagtaggagacctcagtgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactactgcccctggagtagaggagatcagcgggcttccttctggagaagttctagagactactgcccctggagtagatgagatcagtgggcttccttctggagaagttctagagactactgcccctggagtagaggagatcagcgggcttccttctggagaagttctagagacttctacctctgcggtaggggacctcagtggacttccttctggaggagaagttctagagatttctgtctctggagtagaggacatcagtgggcttccttctggagaggttgtagagacttctgcctctggaatagaggatgtcagtgaacttccttcaggagaaggtctagagacctctgcttctggagtagaggacctcagcaggctcccttctggagaagaagttctagagatttctgcctctggatttggggacctcagtggagttccttctggaggagaaggtctagagacctctgcttctgaagtagggactgacctcagtgggcttccttctggaagggagggtctagagacttcagcttctggagctgaggacctcagtgggttgccttctggaaaagaagacttggtggggtcagcttctggagacttggacttgggcaaactgccttctggaactctaggaagtgggcaagctccagaaacaagtggtcttccctctggatttagtggtgagtattctggggtggaccttggaagtggcccaccctctggcctgcctgactttagtggacttccatctggattcccaactgtttccctagtggattctacattggtggaagtggtcacagcctccactgcaagtgaactggaagggaggggaaccattggcatcagtggtgcaggagaaatatctggactgccctccagtgagctggacattagtgggagagctagtggactcccttcaggaactgaactcagtggccaagcatctgggtctcctgatgtcagtggggaaatacctggactctttggtgtcagtggacagccatcagggtttcctgacactagtggggaaacatctggagtgactgagcttagcgggctgtcctctggacaaccaggtgttagtggagaagcatctggagttctttatggcactagtcaaccctttggcataactgatctgagtggagaaacatctggggtccctgatctcagtgggcagccttcagggttaccagggttcagtggggcaacatcaggagtccctgacctggtttctggtaccacgagtggcagcggtgaatcttctgggattacatttgtggacaccagtttggttgaagtggcccctactacatttaaagaagaagaaggcttagggtctgtggaactcagtggcctcccttccggagaggcagatctgtcaggcaaatctgggatggtggatgtcagtggacagttttctggaacagtcgattccagtgggtttacatcccagactccggaattcagtggcctaccaagtggcatagctgaggtcagtggagaatcctccagagctgagattgggagcagcctgccctcgggagcatattatggcagtggaactccatctagtttccccacggtctctcttgtagacagaactttggtggaatctgtaacccaggctccaacagcccaagaggcaggagaagggccttctggcattttagaactcagtggtgctcattctggagcaccagacatgtctggggagcattctggatttctggacctaagtgggctgcagtccgggctgatagagcccagcggagagccaccaggtactccatattttagtggggattttgccagcaccaccaatgtaagtggagaatcctctgtagccatgggcaccagtggagaggcctcaggacttccagaagttactttaatcacttctgagttcgtggagggtgttactgaaccaactatttctcaggaactaggccaaaggccccctgtgacacacacaccccagctttttgagtccagtggaaaagtctccacagctggggacattagtggagctaccccagtgctccctgggtctggagtagaagtatcatcagtcccagaatctagcagtgagacgtccgcctatcctgaagctgggttcggggcatctgccgcccctgaggccagcagagaagattctgggtcccctgatctgagtgaaaccacctctgcattccacgaagctaaccttgagagatcctctggcctaggagtgagcggcagcactttgacatttcaagaaggcgaggcgtccgctgccccagaagtgagtggagaatccaccaccaccagtgatgtggggacagaggcaccaggcttgccttcagccactcccacggcttctggagacaggactgaaatcagcggagacctgtctggtcacacctcgcagctgggcgttgtcatcagcaccagcatcccagagtctgagtggacccagcagacccagcgccctgcagagacgcatctagaaattgagtcctcaagcctcctgtactcaggagaagagactcacacagtcgaaacagccacctccccaacagatgcttccatcccagcttctccggaatggaaacgtgaatcagaatcaactgctgcagaccaggaggtatgtgaggagggctggaacaagtaccagggccactgttaccgccacttcccggaccgcgagacctgggtggatgctgagcgccggtgtcgggagcagcagtcacacctgagcagcatcgtcacccccgaggagcaggagtttgtcaacaacaatgcccaagactaccagtggatcggcctgaacgacaggaccatcgaaggggacttccgctggtcagatggacaccccatgcaatttgagaactggcgccccaaccagcctgacaacttttttgccgctggagaggactgtgtggtgatgatctggcacgagaagggcgagtggaatgatgttccctgcaattaccacctccccttcacgtgtaaaaagggcacagccaccacctacaaacgcagactacagaagcggagctcacggcaccctcggaggagccgccccagcacagcccactgagaagagcttccaggacgcacccaggacgctgagcccaggagcctgccaggctgacgtgcatcccacccagacggtgtcctcttcttgtcgctttttgtcatataaggaatcccattaaaaaaaaaa

SEQ ID NO: 18 - protein, 2316 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGHVYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACPGTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAETEESTEGPSATEVPSASEEPSPSEVPFPSEEPSPSEEPFPSVRPFPSVELFPSEEPFPSKEPSPSEEPSASEEPYTPSPPEPSWTELPSSGEESGAPDVSGDFTGSGDVSGHLDFSGQLSGDRASGLPSGDLDSSGLTSTVGSGLTVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGVPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGVSGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAADQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTATTYKRRLQKRSSRHPRRSRPSTAH

SEQ ID NO: 19 - DNA, 2722 bp, Homo sapiens
caacagtccccaggcatcaccattcaagatgcatccaggggtcctggctgccttcctcttcttgagctggactcattgtcgggccctgccccttcccagtggtggtgatgaagatgatttgtctgaggaagacctccagtttgcagagcgctacctgagatcatactaccatcctacaaatctcgcgggaatcctgaaggagaatgcagcaagctccatgactgagaggctccgagaaatgcagtctttcttcggcttagaggtgactggcaaacttgacgataacaccttagatgtcatgaaaaagccaagatgcggggttcctgatgtgggtgaatacaatgttttccctcgaactcttaaatggtccaaaatgaatttaacctacagaattgtgaattacacccctgatatgactcattctgaagtcgaaaaggcattcaaaaaagccttcaaagtttggtccgatgtaactcctctgaattttaccagacttcacgatggcattgctgacatcatgatctcttttggaattaaggagcatggcgacttctacccatttgatgggccctctggcctgctggctcatgcttttcctcctgggccaaattatggaggagatgcccattttgatgatgatgaaacctggacaagtagttccaaaggctacaacttgtttcttgttgctgcgcatgagttcggccactccttaggtcttgaccactccaaggaccctggagcactcatgtttcctatctacacctacaccggcaaaagccactttatgcttcctgatgacgatgtacaagggatccagtctctctatggtccaggagatgaagaccccaaccctaaacatccaaaaacgccagacaaatgtgacccttccttatcccttgatgccattaccagtctccgaggagaaacaatgatctttaaagacagattcttctggcgcctgcatcctcagcaggttgatgcggagctgtttttaacgaaatcattttggccagaacttcccaaccgtattgatgctgcatatgagcacccttctcatgacctcatcttcatcttcagaggtagaaaattttgggctcttaatggttatgacattctggaaggttatcccaaaaaaatatctgaactgggtcttccaaaagaagttaagaagataagtgcagctgttcactttgaggatacaggcaagactctcctgttctcaggaaaccaggtctggagatatgatgatactaaccatattatggataaagactatccgagactaatagaagaagacttcccaggaattggtgataaagtagatgctgtctatgagaaaaatggttatatctattttttcaacggacccatacagtttgaatacagcatctggagtaaccgtattgttcgcgtcatgccagcaaattccattttgtggtgttaagtgtctttttaaaaattgttatttaaatcctgaagagcatttggggtaatacttccagaagtgcggggtaggggaagaagagctatcaggagaaagcttggttctgtgaacaagcttcagtaagttatctttgaatatgtagtatctatatgactatgcgtggctggaaccacattgaagaatgttagagtaatgaaatggaggatctctaaagagcatctgattcttgttgctgtacaaaagcaatggttgatgatacttcccacaccacaaatgggacacatggtctgtcaatgagagcataatttaaaaatatatttataaggaaattttacaagggcataaagtaaatacatgcatataatgaataaatcattcttactaaaaagtataaaatagtatgaaaatggaaatttgggagagccatacataaaagaaataaaccaaaggaaaatgtctgtaataatagactgtaacttccaaataaataattttcattttgcactgaggatattcagatgtatgtgcccttcttcacacagacactaacgaaatatcaaagtcattaaagacaggagacaaaagagcagtggtaagaatagtagatgtggcctttgaattctgtttaattttcacttttggcaatgactcaaagtctgctctcatataagacaaatattcctttgcatattataaaggataaagaaggatgatgtctttttattaaaatatttcaggttcttcagaagtcacacattacaaagttaaaattgttatcaaaatagtctaaggccatggcatccctttttcataaattatttgattatttaagactaaaagttgcattttaaccctattttacctagctaattatttaattgtccggtttgtcttggatatataggctattttctaaagacttgtatagcatgaaataaaatatatcttataaagtggaagtatgtatattaaaaaagagacatccaaatttttttttaaagcagtctactagattgtgatcccttgagatatggaaggatgcctttttttctctgcatttaaaaaaatcccccagcacttcccacagtgcctattgatacttggggagggtgcttggcacttattgaatatatgatcggccatcaagggaagaactattgtgctcagagacactgttgataaaaactcaggcaaagaaaatgaaatgcatatttgcaaagtgtattaggaagtgtttatgttgtttataataaaaatatattttcaacagaaaaaaaa

SEQ ID NO: 20 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 21 - DNA, 1973 bp, Homo sapiens
gggatattggagtagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgtttt

SEQ ID NO: 22 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 23 - DNA, 3150 bp, Homo sapiens
ccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtc

SEQ ID NO: 24 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 25 - DNA, 1666 bp, Homo sapiens
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 26 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 27 - DNA, 1125 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattc

SEQ ID NO: 28 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 29 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 30 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 31 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 32 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 33 - DNA, 42 bp, Artificial Sequence
  primer
cttcgtcgggaccacttcgggcaggagtcgcgtggcgaaggc

SEQ ID NO: 34 - protein, 49 aa, Homo sapiens
SNWSPWSACSSSTCDKGKRMRQRMLKAQLDLSVPCPDTQDFQPCMGPGC

SEQ ID NO: 35 - protein, 49 aa, Homo sapiens
MSEWITWSPCSISCGMGMRSRERYVKQFPEDGSVCTLPTEETEKCTVNE

SEQ ID NO: 36 - protein, 51 aa, Homo sapiens
MTEWGEWDECSATCGMGMKKRHRMIKMNPADGSMCKAETSQAEKCMMPECH

SEQ ID NO: 37 - protein, 49 aa, Homo sapiens
SPWSEWSDCSVTCGKGMRTRQRMLKSLAELGDCNEDLEQVEKCMLPECP

SEQ ID NO: 38 - protein, 49 aa, Homo sapiens
TEWSQWSECNKSCGKGHVIRTRMIQMEPQFGGAPCPETVQRKKCRIRKC

SEQ ID NO: 39 - protein, 48 aa, Homo sapiens
WTAWSECTKLCGGGIQERYMTVKKRFKSSQFTSCKDKKEIRACNVHPC

SEQ ID NO: 40 - protein, 23 aa, Homo sapiens
MRLSPAPLKLSRTPALLALALPL

SEQ ID NO: 41 - DNA, 24 bp, Artificial Sequence
  forward primer
agtgctgtcattgatctcattgga

SEQ ID NO: 42 - DNA, 25 bp, Artificial Sequence
  reverse primer
tcagaggaatagagaccattggatt

SEQ ID NO: 43 - DNA, 20 bp, Artificial Sequence
  forward primer
gccgtgacctcagacttagc

SEQ ID NO: 44 - DNA, 20 bp, Artificial Sequence
  reverse primer
ttttgtccttggggttcttg

SEQ ID NO: 45 - DNA, 20 bp, Artificial Sequence
  forward primer
aacatgttgaacatctcccc

SEQ ID NO: 46 - DNA, 20 bp, Artificial Sequence
  reverse primer
atctgcagacagacgctggc

SEQ ID NO: 47 - DNA, 58 bp, Artificial Sequence
  shRNA sequence
ccgggccaagtacagactcacatttctcgagaaatgtgagtctgtacttggctttttg

SEQ ID NO: 48 - DNA, 58 bp, Artificial Sequence
  shRNA sequence
ccggcagagttgtcatcgagagaatctcgagattctctcgatgacaactctgtttttg

SEQ ID NO: 49 - DNA, 58 bp, Artificial Sequence
  shRNA sequence
ccgggcagaacttggagactgcaatctcgagattgcagtctccaagttctgctttttg

SEQ ID NO: 50 - DNA, 58 bp, Artificial Sequence
  shRNA sequence
ccggcctgacaatgtcgatgatattctcgagaatatcatcgacattgtcaggtttttg

SEQ ID NO: 51 - DNA, 58 bp, Artificial Sequence
  shRNA sequence
ccgggctggattatttgcttgtttactcgagtaaacaagcaaataatccagctttttg

SEQ ID NO: 52 - DNA, 58 bp, Artificial Sequence
  shRNA sequence
ccgggacctatgagtcaccaaacaactcgagttgtttggtgactcataggtctttttg

SEQ ID NO: 53 - DNA, 58 bp, Artificial Sequence
  shRNA sequence
ccgggcgctacatgactgtgaagaactcgagttcttcacagtcatgtagcgctttttg

SEQ ID NO: 54 - DNA, 58 bp, Artificial Sequence
  shRNA sequence
ccgggccaagtacagactcacgtttctcgagaaacgtgagtctgtacttggctttttg

SEQ ID NO: 55 - DNA, 58 bp, Artificial Sequence
  shRNA sequence
ccggcgtgacctatgagtcaccaaactcgagtttggtgactcataggtcacgtttttg

SEQ ID NO: 56 - DNA, 58 bp, Artificial Sequence
  shRNA sequence
ccgggcaggtgatgatggctactttctcgagaaagtagccatcatcacctgctttttg

(19) United States
(12) Patent Application Publication
(10) Pub. No.: US 2014/0274766 A1
(43) Pub. Date: Sep. 18, 2014

(54) METHODS OF USING F-SPONDIN AS A BIOMARKER FOR CARTILAGE DEGENERATIVE CONDITIONS AND BONE DISEASES

(71) Applicants: Steven B. Abramson, Rye, NY (US); Mukundan Attur, Woodside, NY (US); Glyn Palmer, Brooklyn, NY (US); Ashok Amin, Union, NY (US)

(72) Inventors: Steven B. Abramson, Rye, NY (US); Mukundan Attur, Woodside, NY (US); Glyn Palmer, Brooklyn, NY (US); Ashok Amin, Union, NY (US)

(21) Appl. No.: 14/155,491

(22) Filed: Jan. 15, 2014

Related U.S. Application Data
(63) Continuation of application No. 12/455,296, filed on May 29, 2009, now abandoned.

(51) Int. Cl. C12Q 1/68; G01N 33/50; G01N 33/68

(52) U.S. Cl. CPC C12Q 1/6883; G01N 33/6893; G01N 33/6887; G01N 33/5041
USPC 506/9; 435/6.12; 435/6.11; 435/7.92; 436/501; 435/7.93; 435/7.94; 435/29; 435/7.4; 435/6.13

(57) ABSTRACT

Methods for identifying subjects having, or at risk for developing, osteoarthritis or other cartilage degenerative conditions by measuring levels of expression of F-spondin are provided. Assays, kits and methods for determining and assaying the presence of F-spondin in individual patients are disclosed. Oligonucleotide probes and primers for use in the assays, kits and methods are described. Assays and methods are disclosed for identifying candidate compounds that modulate F-spondin levels of expression and/or function or for determining and evaluating an individual's response to drugs and therapeutic agents, are provided. The invention further relates to the modulation of F-spondin, particularly the inhibition of F-spondin, for increasing or stimulating bone formation and/or growth and mediating the alleviation of bone disease, disorders or conditions. The use of F-spondin, an active fragment thereof, or a modulator thereof for enhancing cartilage repair or preventing or treating cartilage degeneration, degenerative diseases or arthritic conditions is also provided.

### CROSS-REFERENCE TO RELATED APPLICATIONS

The present application is a continuation-in-part application claiming the priority of copending application PCT/US2007/024658, filed Nov. 30, 2007, which in turn claims priority from provisional application Ser. No. 60/631,828, filed Nov. 30, 2004, the disclosures of which are incorporated by reference herein in their entirety. Applicants claim the benefits of 35 U.S.C. §120 as to the PCT application and priority under 35 U.S.C. §119 as to the provisional application.

### FIELD OF THE INVENTION

The invention relates to the identification and use of F-spondin as a biomarker for chondrocyte hypertrophy and more specifically, as a biomarker for osteoarthritis and other cartilage degenerative conditions. The invention further relates to the identification of F-spondin as involved in endochondral bone formation and growth and the modulation of F-spondin, particularly the inhibition of F-spondin, for increasing or stimulating bone formation and/or growth and mediating the alleviation of bone disease, disorders or conditions. The invention provides methods of screening, diagnosing or prognosing a cartilage degenerative condition in a subject by measuring the levels of the F-spondin gene or gene product in a tissue sample from the subject. Polynucleotides and proteins which specifically and/or selectively hybridize to F-spondin are also encompassed within the scope of the invention, as are kits containing said polynucleotides and proteins for use in diagnosing individuals as having a cartilage degenerative condition. The polynucleotides and proteins which specifically and/or selectively hybridize to F-spondin and kits containing said polynucleotides and proteins may also be used to monitor disease progression, or for monitoring the efficacy of therapeutic regimens. The invention also provides methods of using the F-spondin gene, or gene product, in the identification of compounds that bind to and/or modulate the expression and/or activity/function of the F-spondin gene. F-spondin and/or the candidate compounds identified by these methods can then be used for the prevention, treatment, management and/or amelioration of osteoarthritis, or a symptom thereof, as well as other cartilage degenerative conditions and in cartilage repair, and bone diseases or disorders.

### BACKGROUND OF THE INVENTION

While arthritis encompasses over 120 diseases and conditions that affect joints, the surrounding tissues, and other connective tissues, the most common types are osteoarthritis and rheumatoid arthritis. Arthritis involves both cartilage breakdown and new bone formation, and in many cases, may lead to the loss of joint function. More particularly, osteoarthritis (OA) is a chronic disease in which the articular cartilage, which is located on the end of a bone, and which forms the articulating surface of the joints, gradually degenerates over time. Articular cartilage, which is predominantly composed of chondrocytes, type II collagen, proteoglycans and water, has no blood or nerve supply. Chondrocytes are the cells that are responsible for manufacturing the type II collagen and proteoglycans, both of which form the cartilage matrix. The cartilage matrix has physical-chemical properties that allow for saturation of the matrix with water. In the absence of osteoarthritis, articular cartilage has exceptional wear characteristics and allows for almost frictionless movement between the articulating cartilage surfaces.

Chondrogenesis is the earliest well-orchestrated and controlled phase of skeletal development, involving mesenchymal cell recruitment and migration, condensation of progenitors, chondrocyte proliferation and differentiation, and maturation. This process is controlled exquisitely by cellular interactions with the growth factors, surrounding matrix proteins and other environmental factors that mediate cellular signaling pathways and transcription of specific genes in a temporal-spatial manner. Production of and response to different growth factors are observed at all times and autocrine and paracrine cell stimulations are key elements of the process. Particularly relevant is the role of the TGF-beta superfamily, and more specifically of the BMP subfamily. Other factors include retinoids, FGFs, GH, and IGFs. The growing evidences demonstrated that complicated cellular signaling language and informational content of chondrogenesis lie, not in an individual growth factor, but in the entire set of growth factors and others signals to which a cell is exposed. The ways in which growth factors exert their combinatorial effects are becoming clearer as the molecular mechanisms of growth factors actions are being investigated. Gene- and cell-based therapy of growth factors for cartilage disorders are under intensive study. The isolation of the growth factor(s) that regulating chondrogenesis is therefore of great importance from both a pathophysiological and a therapeutic standpoint.

There are many factors that may increase an individual's risk for developing osteoarthritis (OA). These include age, female gender, joint injury or overuse, obesity, joint misalignment, hereditary gene defects, accidental or athletic trauma, surgery, drugs and heavy physical demand, as well as other diseases that change the normal structure and function of cartilage. If an individual is experiencing swelling or stiffness in a joint for more than two weeks, arthritis is suspected and it is advisable to have a physician to assess whether OA is the cause of the discomfort, or whether there are other underlying problems that manifest themselves in a manner similar to OA. A series of X-rays may be taken to help in the diagnosis and these may be repeated over time to determine the progress of the disease, in particular, the progression of joint damage. Such x-rays of the affected joints can show cartilage loss, bone damage, and bone spurs. A needle aspirate of synovial fluid from the affected joint may be obtained to rule out an infection in the joint.

While the procedures noted above appear to be only modestly invasive, it would be advantageous to identify one or more biomarkers of OA or other cartilage degenerative conditions that could be used to assess various aspects of disease activity (Chevalier, X. (1997) Rev. Rhum. Engl. Ed. 64: 562-577). Current research in this area has identified a number of possible surrogate biomarkers of arthritis, which may reflect metabolic changes in the joint associated with cartilage destruction and remodeling. These include hyaluronate, cartilage oligomeric matrix protein, keratan sulfate, metalloproteinase activity, and various cytokines (Lohmander, L. S. et al. (2005), J. Rheumatol. June 32(6):1142-1143; Lohmander, L. S. et al. (1999), Arthritis Rheum, March, 42(3):534-544; Lohmander, L. S. et al. (1998), Osteoarthritis Cartilage, September 6(5):351-361; Panula, H. E. et al. (1998), Acta Orthop. Scand. April 69(2): 152-158; Lohmander, L. S. et al, (1997), J. Rheumatol. April 24(4):782-785; Lohmander, L. S. (1997) Baillieres Clin. Rheumatol. 11: 711-726; see also U.S. 20050221383).

Many studies are now being done to determine the mediators contributing to cartilage remodeling. However, to date, there is only limited knowledge of the underlying molecular mechanisms that play a role in the initiation and progression of arthritis. On the other hand, at a cellular level, the articular chondrocyte appears to play a key role in both cartilage breakdown, as well as, new bone growth, and this is likely to be reflected in changes in gene expression and cellular transcriptional activity. Whether the change in level of expression of any one or more genes in the chondrocyte, synovial fluid, or other body fluids can be used as a surrogate biomarker for osteoarthritis or other cartilage degenerative conditions remains to be seen. Accordingly, the understanding of the dysregulation of chondrocyte function, phenotype and extracellular matrix (ECM) interactions in osteoarthritis has been a major focus of investigation (Attur M G, Dave M N, Stuchin S, Kowalski A J, Steiner G, Abramson S B, et al. Osteopontin: an intrinsic inhibitor of inflammation in cartilage. Arthritis Rheum 2001; 44(3):578-84; Okazaki K, Yu H, Davies S R, Imamura T, Sandell L J. A promoter element of the CD-RAP gene is required for repression of gene expression in non-cartilage tissues in vitro and in vivo. J Cell Biochem 2006; 97(4):857-68; Attur M G, Dave M N, Clancy R M, Patel I R, Abramson S B, Amin A R. Functional genomic analysis in arthritis-affected cartilage: yin-yang regulation of inflammatory mediators by alpha 5 beta 1 and alpha V beta 3 integrins. J Immunol 2000; 164(5):2684-91; Attur M G, Dave M, Cipolletta C, Kang P, Goldring M B, Patel I R, et al. Reversal of autocrine and paracrine effects of interleukin 1 (IL-1) in human arthritis by type II IL-1 decoy receptor. Potential for pharmacological intervention. J Biol Chem 2000; 275 (51):40307-15; Homandberg G A. Cartilage damage by matrix degradation products: fibronectin fragments. Clin Orthop Relat Res 2001 (391 Suppl):S100-7; Attur M G, Dave M N, Tsunoyama K, Akamatsu M, Kobori M, Miki J, et al. “A system biology” approach to bioinformatics and functional genomics in complex human diseases: arthritis. Curr Issues Mol Biol 2002; 4(4):129-46).

In osteoarthritis (OA), synovium, cartilage and bone are each sites of increased cytokine, growth factor and inflammatory mediator production that are believed to contribute to disease pathogenesis (Petersson I F, Boegard T, Svensson B, Heinegard D, Saxne T. Changes in cartilage and bone metabolism identified by serum markers in early osteoarthritis of the knee joint. Br J Rheumatol 1998; 37(1):46-50; Reginster J P, J P; Martel-Pelletier, J; Henrotin, Y. Genetic and metabolic aspects. Berlin: Springer-Verlag; 1999). The role of bone in OA is of great interest, yet remains poorly understood (Reginster. J P, J P; Martel-Pelletier, J; Henrotin, Y. Genetic and metabolic aspects. Berlin: Springer-Verlag; 1999). Osteophyte formation and subchondral bone remodeling are early features of OA and appear to result from the local production of anabolic growth factors, such as insulin-like growth factor (IGF)-1 and transforming growth factor (TGF)-β Significant increase in bone turnover occurs in subchondral regions and biomarkers such as urine N-terminal cross linking telopeptides of type 1 collagen, a marker of type II collagen degradation indicative of bone resorption, are elevated in patients with knee OA. Synovial involvement is also recognized as an important feature of osteoarthritis. Arthroscopy has demonstrated localized synovial proliferative and inflammatory changes in up to 50% of patients with knee OA (Ayral X. Diagnostic and quantitative arthroscopy: quantitative arthroscopy. Baillieres Clin Rheumatol 1996; 10 (3):477-94). Proteases and cytokines produced by activated synovium have been suggested to accelerate deterioration of contiguous cartilage lesions (Ayral X. Diagnostic and quantitative arthroscopy: quantitative arthroscopy. Baillieres Clin Rheumatol 1996; 10 (3):477-94).

Although both bone and synovium have important roles in the pathogenesis of OA, most interest in disease modifying treatments has focused on molecular events within articular cartilage. OA chondrocytes undergo a series of complex changes, including hypertrophy, proliferation, catabolic alteration and, ultimately, death. The regulation of these phenotypic changes at different stages of disease is under intensive study, with focus on the biomechanical and biochemical signals that regulate each of these discrete chondrocyte responses (Petersson I F, Boegard T, Svensson B, Heinegard D, Saxne T. Changes in cartilage and bone metabolism identified by serum markers in early osteoarthritis of the knee joint. Br J Rheumatol 1998; 37(1):46-50; Ayral X, Gicquere C, Duhalde A, Boucheny D, Dougados M. Effects of video information on preoperative anxiety level and tolerability of joint lavage in knee osteoarthritis. Arthritis Rheum 2002; 47(4):380-2). Chondrocytes themselves are featured protagonists in this cascade of change, not only the target of external biomechanical and biochemical stimuli, but also themselves the cellular source of cytokines, chemokines, proteases and inflammatory mediators that promote the deterioration of articular cartilage (Petersson I F, Boegard T, Svensson B, Heinegard D, Saxne T. Changes in cartilage and bone metabolism identified by serum markers in early osteoarthritis of the knee joint. Br J Rheumatol 1998; 37(1):46-50; Reginster J P, J P; Martel-Pelletier, J; Henrotin, Y. Genetic and metabolic aspects. Berlin: Springer-Verlag; 1999). Pathogenic molecules produced by OA chondrocytes include matrix metalloproteinases (MMPs), interleukin (IL)-1, tumor necrosis factor (TNF), IL-6, IL-8, nitric oxide, prostaglandins and leukotrienes (Petersson I F, Boegard T, Svensson B, Heinegard D, Saxne T. Changes in cartilage and bone metabolism identified by serum markers in early osteoarthritis of the knee joint. Br J Rheumatol 1998; 37(1):46-50; Ayral X, Gicquere C, Duhalde A, Boucheny D, Dougados M. Effects of video information on preoperative anxiety level and tolerability of joint lavage in knee osteoarthritis. Arthritis Rheum 2002; 47(4):380-2). There is also evidence that OA chondrocytes exhibit increased anabolic activity, including increased synthesis of type II collagen, proteoglycan and other extracellular matrix proteins, as well as the expression of genes associated with the chondroprogenitor hypertrophic phenotype (Lippiello L, Hall D, Mankin H J. Collagen synthesis in normal and osteoarthritic human cartilage. J Clin Invest 1977; 59(4):593-600; Aigner T, Zhu Y, Chansky H H, Matsen F A, 3rd, Maloney W J, Sandell L J. Reexpression of type IIA procollagen by adult articular chondrocytes in osteoarthritic cartilage. Arthritis Rheum 1999; 42(7):1443-50; Sandell L J, Aigner T. Articular cartilage and changes in arthritis. An introduction: cell biology of osteoarthritis. Arthritis Res 2001; 3 (2):107-13).

TGF-β appears to play a dual role, involved both in anabolic repair processes, while at the same time promoting osteophyte formation. TGF-β suppresses expression of matrix metalloproteinase-13 and matrix metalloproteinase-9, and proinflammatory cytokines (interleukin-1 β, tumor necrosis factor-α). In contrast, isoforms of TGF-β up-regulate PGES-1 expression and prostaglandin E(2) release (Tchetina E V, Antoniou J, Tanzer M, Zukor D J, Poole A R. Transforming growth factor-beta2 suppresses collagen cleavage in cultured human osteoarthritic cartilage, reduces expression of genes associated with chondrocyte hypertrophy and degradation, and increases prostaglandin E(2) production. Am J Pathol 2006; 168(1):131-40).

Cartilage extracellular matrix is essential for the maintenance of chondrocyte differentiation and function. In addition to maintaining cell adhesion, cell-matrix interaction, which includes transmembrane signaling that integrates the cell with its external environment, regulating responses to growth factors, cytokines and mechanical stress (Chowdhury T T, Appleby R N, Salter D M, Bader D A, Lee D A. Integrin-mediated mechanotransduction in IL-1beta stimulated chondrocytes. Biomech Model Mechanobiol 2006). Normally the remodeling of articular matrix is highly regulated to maintain a balance of distinct macromolecular components. In osteoarthritis, there are characteristic changes in the ECM, including early depletion of proteoglycan due to the production of matrix metalloproteinases (MMPs), aggrecanases (ADAMTSs) and other proteases, which results in the mechanical loss of tissue resilience. Less well understood is the role of increased expression of ECM proteins in OA such as fibronectin, osteopontin, decorin, cartilage oligomeric matrix protein (COMP) and fibromodulin; these ECM proteins are likely to interact with articular chondrocytes through cell-surface receptors and further alter chondrocyte metabolism in disease (Attur M G, Dave M N, Clancy R M, Patel I R, Abramson S B, Amin A R. Functional genomic analysis in arthritis-affected cartilage: yin-yang regulation of inflammatory mediators by alpha 5 beta 1 and alpha V beta 3 integrins. J Immunol 2000; 164(5):2684-91). Moreover, unlike their intact “parent” molecules, degradative fragments of type II collagen, fibronectin and hyaluronan have each been shown to stimulate catabolic activity of chondrocytes, effects which are postulated to amplify disease progression in OA (Loeser R F. Molecular mechanisms of cartilage destruction: mechanics, inflammatory mediators, and aging collide. Arthritis Rheum 2006; 54(5):1357-60).

“F.-Spondin” (“Floor plate” and “thrombospondin” homology; also Spondin-1 and VSGP) is a 110 kDa, secreted, heparin-binding extracellular matrix glycoprotein. F-spondin was first identified as a novel protein secreted by neuronal cells that caused a rat hippocampal progenitor cell line and primary cortical neural cells to differentiate into cells with the morphological and biochemical features of neurons (Klar A, Baldassare M, Jessell T M. F-spondin: a gene expressed at high levels in the floor plate encodes a secreted protein that promotes neural cell adhesion and neurite extension. Cell 1992; 69(1):95-110). It is a member of a family of proteins that collectively belong to a subgroup of TSR (thrombospondin) type I class molecules, which include COMP, CTGF, ADAMTS-7&12 and CILP (Feinstein Y, Klar A. The neuronal class 2 TSR proteins F-spondin and Mindin: a small family with divergent biological activities. Int J Biochem Cell Biol 2004; 36(6):975-80; Tucker R P. The thrombospondin type 1 repeat superfamily. Int J Biochem Cell Biol 2004; 36(6):969-74). Human F-Spondin is synthesized as an 807 amino acid (aa) precursor that contains a 28 aa signal sequence and a 779 aa mature region. As depicted in FIG. 9, the mature region includes an N-terminal reelin-like domain (aa 1-200), a centrally placed F-spondin (FS) type segment (aa 201-440), and six C-terminal class 2 thrombospondin type I repeats. Class 1 and 2 repeats differ in the placement of their cysteine residues. The fifth and sixth TSP repeats (aa 668-806) apparently bind ECM, while TSP repeats 1-4 (aa 442-666), plus the spondin segment, are suggested to mediate either repulsive activity (on motor neurons), or outgrowth promoting activity (on sensory neurons).

At least two isoforms of F-spondin are known. Both are proteolytically-generated, one by plasmin, another by an unidentified protease. Plasmin cleaves the C-terminus at two points, generating a soluble, 95 kDa, 656 aa F-spondin that contains all but TSP repeats number 5 and 6. The unidentified protease appears to cleave F-Spondin between the FS segment and the first TSP repeat, generating 60 kDa and 50 kDa fragments, respectively (Tzarfaty-Majar V, Lopez-Alemany R, Feinstein Y, Gombau L, Goldshmidt O, Soriano E, et al. Plasmin-mediated release of the guidance molecule F-spondin from the extracellular matrix. J Biol Chem 2001; 276(30):28233-41). Experiments with various deletion variants of F-spondin have further demonstrated that plasmin releases the ECM-bound F-spondin protein.

Mature human F-spondin exhibits 98%, 97%, 98%, and 97% amino acid identity to mature canine, rat, bovine and mouse F-Spondin, respectively. Axotomy of adult sciatic nerve causes massive upregulation of F-spondin (Burstyn-Cohen T, Frumkin A, Xu Y T, Scherer S S, Klar A. Accumulation of F-spondin in injured peripheral nerve promotes the outgrowth of sensory axons. J Neurosci 1998; 18(21):8875-85). Mammalian cells known to express F-spondin include floor plate epithelium, ventral motor neurons, Schwann cells, fibroblasts, hippocampal pyramidal cells, endothelial cells, vascular smooth muscle cells and some tumor cells. Recombinant F-spondin stimulates proliferation of vascular smooth muscle cells, suggesting that, F-spondin also acts on non-neuronal cells via unidentified receptor. In human endothelial cells F-spondin has been shown to inhibit angiogenesis via alphaV beta3 interactions and this interaction is RGD independent. Thus, f-spondin may interact with multiple receptors and activates various signaling pathways (Terai Y, Abe M, Miyamoto K, Koike M, Yamasaki M, Ueda M, et al. Vascular smooth muscle cell growth-promoting factor/F-spondin inhibits angiogenesis via the blockade of integrin alphavbeta3 on vascular endothelial cells. J Cell Physiol 2001; 188(3):394-402).

In addition to its ability to promote neurite outgrowth and inhibit angiogenesis, F-spondin interacts with other proteins. For example, through co-immunoprecipitation experiments, Hoe et al., demonstrated that F-spondin interacts with an apoE receptor (apoE receptor 2 [ApoEr2]) through the thrombospondin domain of F-spondin and the ligand binding domain of ApoEr2. Full-length F-spondin, but none of the individual F-spondin domains, increased cleavage of APP and ApoEr2, resulting in more secreted forms of APP and ApoEr2 and more C-terminal fragments (CTF) of these proteins (Hoe H S, Wessner D, Beffert U, Becker A G, Matsuoka Y, Rebeck G W. F-spondin interaction with the apolipoprotein E receptor ApoEr2 affects processing of amyloid precursor protein. Mol Cell Biol 2005; 25(21):9259-68).

Thus, while the use of genomic or proteomic approaches for the understanding of the complexities of arthritis, as well as other cartilage degenerative conditions, promises to yield significant advances in the diagnosis and treatment of these diseases, there is still a need for a simple non-invasive diagnostic test for detecting the presence of osteoarthritis and other cartilage degenerative conditions and a prognostic test that effectively monitors a patient's response to therapy. Further, there remains a need for improved and effective therapies in treatment and prevention of arthritis and cartilage degenerative conditions, and for enhancement and promotion of cartilage repair. These needs are addressed by the present invention.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

In its broadest aspect, the present invention is based on the identification of enhanced levels of F-spondin in osteoarthritic cartilage and synovium. Further, the identification of F-spondin in articular cartilage and synovial fluid from patients suffering from osteoarthritis (OA) suggests that F-spondin may be used as a biomarker for the screening, diagnosis or prognosis of patients suspected of having OA or other cartilage degenerative conditions. The recognition and identification of factor(s) that regulate chondrogenesis and chondrocyte maturation is of great importance from both a pathophysiological and a therapeutic standpoint. Thus, one purpose of this invention is to utilize F-spondin, including fragments thereof, agonists and antagonists, and modulators thereof, that are for normal cartilage development and progression of cartilage disorders, including arthritis, to further understand chondrogenesis and cartilage degeneration and to provide new molecular targets for prediction, diagnosis and treatment of cartilage-related diseases. OA chondrocytes have been shown to express markers associated with growth plate chondrocyte maturation, and the present invention is further based on the recognition that F-spondin is expressed in embryonic growth plate cartilage and enhances the expression of chondrocyte maturation markers. Thus, F-spondin may be used to stimulate and enhance chondrocyte maturation, particularly in conditions where further or enhanced maturation is desired, such as in cartilage degeneration, cartilage repair, including as an adjunct to cell repair therapies and as a preventative after cartilage injury.

Accordingly, methods are proposed for determining the presence of OA or a cartilage degenerative condition in a subject, or for monitoring cartilage repair, or for assessing the risk of developing OA or a cartilage degenerative condition, or for determining a patient's response to therapies through use of F-spondin as a biomarker for these conditions. Based on these identifications, the present invention provides methods of detecting F-spondin as well as reagents needed to accomplish this task. The invention specifically provides nucleotide probes for detecting the F-spondin gene and antibodies for detecting the proteins encoded by this gene, and methods of detecting the F-spondin gene or gene product in a sample, methods of determining a risk of having or developing a disorder associated with the presence of the F-spondin gene, methods of screening for candidate compounds used to treat cartilage disorders associated with the presence of the F-spondin gene, methods of treating cartilage disorders associated with the presence of the F-spondin gene, and methods of using the probes and antibodies of the present invention for detection of OA or other related cartilage degenerative conditions.

In related aspects, the inventors have demonstrated that F-spondin regulates articular cartilage metabolism and is involved in chondrocyte maturation. Further, the present invention demonstrates that F-spondin plays a role in endochondral bone formation and growth. Using chondrocyte maturation models and bone culture systems, F-spondin is shown to be upregulated during chondrocyte maturation, is a late stage marker of chondrocyte terminal differentiation, and regulates bone growth, particularly endochondral bone growth. Modulation of F-spondin has application in both degenerative diseases of articular cartilage and tracheal cartilage or other permanent cartilage structures and in transient cartilage or at sites or under conditions wherein cartilage is resorbed and replaced with bone. Thus, in embryonic cartilaginous skeleton, the epiphyseal growth plates of long bones, the cartilaginous callus formed at fracture sites, and the tissue created during distraction osteogenesis F-spondin and modulation thereof may be utilized. F-spondin therefore has application in situations or under conditions wherein cartilage is replaced by bone or bone growth is warranted or necessary. Modulation of F-spondin may be used in the modulation, alleviation or treatment of diseases of the transient growth cartilage of the long bones, including chondrodysplasias and dwarfism, on in bone diseases, bone degeneration, bone fractures or bone cancer where replacement of bone or endochondral bone formation is warranted or helpful.

Accordingly, a first aspect of the invention provides a method for screening, diagnosing or prognosing a cartilage degenerative condition in a subject, or for measuring cartilage degeneration resulting from ageing, trauma or a sports related injury in a subject, or for monitoring the state of chondrocyte cell transplant to a lesioned area, wherein said condition or said cartilage degeneration is characterized by an increase in the level of expression of F-spondin, said method comprising:

(I) measuring an amount of an F-spondin gene or gene product, or a fragment thereof, in a tissue sample obtained from the subject, wherein said F-spondin gene or gene product is:

(a) a DNA corresponding to any one of SEQ ID NOs: 1, 3 or 5 or a nucleic acid derived therefrom;

(b) a protein comprising any one of SEQ ID NOs: 2, 4 or 6;

(c) a nucleic acid comprising a sequence hybridizable to any one of SEQ ID NOs: 1, 3 or 5, or their complements under conditions of high stringency, or a protein comprising a sequence encoded by said hybridizable sequence;

(d) a nucleic acid at least 90% homologous to any one of SEQ ID NOs: 1, 3 or 5, or their complements as determined using the NBLAST algorithm; or a protein encoded thereby; and

(II) comparing the amount of said F-spondin gene or gene product in said subject with the amount of F-spondin gene or gene product present in a normal tissue sample obtained from a subject who does not have a cartilage degenerative condition characterized by an increase in the level of expression of F-spondin, or in a predetermined standard, wherein an increase in the amount of said F-spondin gene or gene product in said subject compared to the amount in the normal tissue sample or pre-determined standard indicates the presence of a cartilage degenerative condition in said subject.

In another particular embodiment, the cartilage degenerative condition is selected from the group consisting of osteoarthritis, rheumatoid arthritis, psoriatic arthritis and chondrosarcomas.

A further aspect of the invention provides a method for screening, diagnosing or prognosing a bone condition, disease, disorder or degenerative condition in a subject, or for measuring bone degeneration or endochondral bone formation resulting from fracture, cancer, ageing, trauma or a sports related injury in a subject, wherein said condition, disorder or said degeneration is characterized by an alteration in the level of expression of F-spondin, said method comprising:

(I) measuring an amount of an F-spondin gene or gene product, or a fragment thereof, in a tissue sample obtained from the subject, wherein said F-spondin gene or gene product is:

(a) a DNA corresponding to any one of SEQ ID NOs: 1, 3 or 5 or a nucleic acid derived therefrom;

(b) a protein comprising any one of SEQ ID NOs: 2, 4 or 6;

(c) a nucleic acid comprising a sequence hybridizable to any one of SEQ ID NOs: 1, 3 or 5, or their complements under conditions of high stringency, or a protein comprising a sequence encoded by said hybridizable sequence;

(d) a nucleic acid at least 90% homologous to any one of SEQ ID NOs: 1, 3 or 5, or their complements as determined using the NBLAST algorithm; or a protein encoded thereby; and

(II) comparing the amount of said F-spondin gene or gene product in said subject with the amount of F-spondin gene or gene product present in a normal tissue sample obtained from a subject who does not have a bone condition, disease, disorder or degenerative condition or in a predetermined standard, wherein an alteration in the amount of said F-spondin gene or gene product in said subject compared to the amount in the normal tissue sample or pre-determined standard indicates the presence of a bone condition, disease, disorder or degenerative condition in said subject.

In another particular embodiment, the measuring of the F-spondin gene or gene product is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assay), immunohistochemistry, a Western blot, a dotblot analysis, a protein microarray, a flow cytometric technique, and proteomics analysis.

In another particular embodiment, the enzyme immunoassay is a competitive assay or a sandwich technique, and wherein antibody binding in combination with a reporter molecule is used to quantify the F-spondin gene product.

In another particular embodiment, the reporter molecule is selected from the group consisting of an enzyme, a fluorophore, a radiolabel, a colored dye, a light absorbing dye, a chemiluminescent molecule and a heavy metal. In another more particular embodiment, the heavy metal is colloidal gold. In another particular embodiment, the proteomics analysis is accomplished by 2-dimensional polyacrylamide gel electrophoresis (2DE) coupled to mass spectrometry (MS).

In another particular embodiment, the tissue sample is selected from the group consisting of whole blood, blood cells, whole blood cell lysates, serum, plasma, urine, bone marrow, cerebrospinal fluid, saliva, chondrocytes, cartilage, bone, osteoblasts, synovium and synovial fluid. In another particular embodiment, the blood cells are selected from the group consisting of white blood cells or red blood cells.

In another particular embodiment, the white blood cells are selected from the group consisting of lymphocytes, monocytes or macrophages, neutrophils, basophils and eosinophils.

In another particular embodiment, the method is used for monitoring the effect of therapy administered to a subject having a cartilage degenerative condition. In another embodiment, the method is used for monitoring the effect of therapy administered to a subject having a bone condition, disease, disorder or degenerative condition.

A second aspect of the invention provides a diagnostic method for determining the predisposition, the onset or the presence of a cartilage degenerative condition in a subject, or the likelihood for developing a cartilage degenerative condition following an injury, in a subject, said method comprising detecting in said subject the existence of a change in the level of the F-spondin gene or gene product, as set forth in any one of SEQ ID NOs: 1, 3 or 5 or in any one of SEQ ID NOs: 2, 4 or 6, respectively, or a fragment thereof, or detecting a polymorphism in the F-spondin gene that affects the function of the protein, said method comprising:
a) obtaining a tissue sample from said subject;
b) permeabilizing the cells in said tissue sample;
c) incubating said tissue sample or cells isolated from said tissue sample with one of the following:
   i) an antibody specific for the F-spondin gene product, or an antibody specific for the gene product of an F-spondin gene having a polymorphism that affects the function of the protein; or
   ii) a nucleic acid probe specific for the F-spondin gene or a nucleic acid probe that hybridizes with an F-spondin gene having a polymorphism that affects the function of the protein;
d) detecting and quantitating the amount of antibody or nucleic acid probe bound;
e) comparing the amount of antibody or nucleic acid probe bound in the tissue sample in said subject to the amount of antibody or nucleic acid probe bound in a normal tissue or cellular sample; and
   wherein the amount of labeled antibody or nucleic acid probe bound correlates directly with the predisposition, the likelihood for developing, or the onset or the presence of a cartilage degenerative condition in said subject.

In one particular embodiment, the cartilage degenerative condition is selected from the group consisting of osteoarthritis, rheumatoid arthritis, psoriatic arthritis, and chondrosarcomas.

In another particular embodiment, the measuring of said F-spondin gene or gene product is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assays), immunohistochemistry, a Western blot, a dotblot analysis, a protein microarray, a flow cytometric technique and proteomics analysis.

In another particular embodiment, the enzyme immunoassay is a competitive assay or a sandwich technique, and wherein antibody binding in combination with a reporter molecule is used to quantify the F-spondin gene product.

In another particular embodiment, the reporter molecule is selected from the group consisting of an enzyme, a fluorophore, a radiolabel, a colored dye, a light absorbing dye, a chemiluminescent molecule and a heavy metal. In a more particular embodiment, the heavy metal is colloidal gold.

In another particular embodiment, the proteomics analysis is accomplished by 2-dimensional polyacrylamide gel electrophoresis (2DE) coupled to mass spectrometry (MS).

In another particular embodiment, the tissue sample is selected from the group consisting of whole blood, blood cells, blood cell lysates, serum, plasma, urine, chondrocytes, cartilage, bone, osteoblasts, synovium and synovial fluid.

In another particular embodiment, the blood cells are selected from the group consisting of white blood cells or red blood cells.

In another particular embodiment, the white blood cells are selected from the group consisting of lymphocytes, monocytes or macrophages, neutrophils, basophils and eosiniphils.

In another particular embodiment, the method is for monitoring the effect of therapy administered to a subject having a cartilage degenerative condition. In a further particular embodiment, the method is for monitoring the effect of therapy administered to a subject having a bone condition, disease, disorder or degenerative condition.

In another particular embodiment, the method is used for evaluating the effectiveness of therapy with an agent useful for treating a cartilage degenerative condition, comprising collecting a series of tissue or cellular samples from a subject suffering from a cartilage degenerative condition, wherein the samples are obtained before the initiation of therapy and during treatment with the agent and measuring the level of F-spondin in the subject before and after the initiation of therapy, wherein a normalization of F-spondin correlates with the effectiveness of therapy with the agent. In another embodiment, the method is used for evaluating the effectiveness of therapy with an agent useful for treating a bone condition, disease, disorder or degenerative condition, comprising collecting a series of tissue or cellular samples from a subject suffering from a bone condition, disease, disorder or degenerative condition, wherein the samples are obtained before the initiation of therapy and during treatment with the agent and measuring the level of F-spondin in the subject before and after the initiation of therapy, wherein a normalization of F-spondin correlates with the effectiveness of therapy with the agent.

In another particular embodiment, the measuring of the F-spondin gene or gene product correlates with a change in the level of expression of at least one gene or gene product, which is a member of the PGE2, active TGF-β in αvβ3 dependent and independent pathways.

In another particular embodiment, the measuring of the F-spondin gene or gene product correlates with an increase in expression of at least one gene or gene product selected from the group consisting of COL2A, aggrecan, MMP-13, BMP2 and PGE2; or with a decrease in expression of at least one gene or gene product selected from the group consisting of MMP-1 and TNF-α; or with activation of latent TGF-β1.

A third aspect of the invention provides a method of measuring chondrocyte hypertrophy in a sample, wherein said hypertrophy is the result of an increase in the level of expression of F-spondin, the method comprising hybridizing a probe comprising the nucleic acid of any one of SEQ ID NOs: 1, 3 or 5, or a portion of at least 15-25 nucleotides thereof, or a full complement thereof, with a nucleic acid from said sample, wherein said hybridizing is indicative of chondrocyte hypertrophy resulting from an increase in the level of expression of F-spondin.

In one particular embodiment, the sample is a human sample. In another particular embodiment, the sample is selected from the group consisting of a non-human primate, a dog, a cat, a rodent, a horse, a cow, a pig, a goat, a sheep, rabbit, guinea pig and any other domestic or non-domestic animal suspected of having OA or a related cartilage degenerative condition or bone condition, disease, disorder or degenerative condition.

In another particular embodiment, the probe is labeled.

In another particular embodiment, the label is selected from the group consisting of a radionuclide, an enzyme, a fluorescent label, a chemiluminescent label, a chromogenic label, and combinations thereof.

In another particular embodiment, the method further comprises evaluating the cartilage degenerative condition using a method selected from the group consisting of X-ray analysis, ultrasound, CT SCAN, MRI or evaluation of synovial fluid aspirate.

In another particular embodiment, the method further comprises evaluating one or more risk factors associated with a cartilage degenerative condition. In another embodiment, the method further comprises evaluating one or more risk factors associated with a bone condition, disease, disorder or degenerative condition.

In another particular embodiment, the one or more risk factors are selected from the group consisting of age, female gender, joint injury or overuse caused by physical labor or sports, obesity, joint alignment, hereditary gene defects, and certain diseases or conditions that may increase the risk of a subject for developing a cartilage degenerative condition.

In another particular embodiment, the diseases or conditions that increase the risk of a subject for developing a cartilage degenerative condition are selected from the group consisting of peripheral neuropathies and neuromuscular disorders that put abnormal stress on a joint. Such neuromuscular disorders may be selected from, but not limited to, muscular dystrophy (ALS), spinal muscular atrophy, diabetes, and post polio syndrome.

A fourth aspect of the invention provides a method of screening for an agent or a candidate compound that blocks or inhibits F-spondin expression or activity/function. In one embodiment, the method comprises:

(a) contacting the F-spondin molecule, or fragments thereof, or cells containing the F-spondin molecule, with an agent or a candidate compound, wherein said F-spondin molecule comprises the nucleic acid sequence of any one of SEQ ID NOs: 1, 3 or 5 and/or the amino acid sequence of any one of SEQ ID NOs: 2, 4 or 6; and

(b) determining the level of F-spondin expression or activity/function in the presence or absence of the agent or candidate compound;

wherein the agent or candidate compound is considered to be effective if the level of F-spondin expression or activity/function is lower in the presence of the agent or candidate compound as compared to in the absence of the agent or candidate compound.

In another particular embodiment, the method further comprises:

(c) measuring the effect of the agent or candidate compound on the level of expression or activity/function of at least one gene or gene product, which is a member of the PGE2, TGF-β or αvβ3 pathways.

In another particular embodiment, the member of the PGE2, TGF-β or αvβ3 pathways is selected from the group consisting of COL2A, aggrecan, MMP-13, BMP2, PGE2, MMP-1, TNF-α, and TGF-β1, and the candidate compound is identified as a positive candidate compound if the expression or activity of one or more molecules selected from the group consisting of COL2A, aggrecan, MMP-13, BMP2 and PGE2 is decreased in the presence, but not the absence of the candidate compound; or if the expression or activity of one or more molecules selected from the group consisting of MMP-1 and TNF-α is increased in the presence, but not the absence of the candidate compound; or if activation of latent TGF-β1 is inhibited in the presence, but not the absence of the candidate compound.

A fifth aspect of the invention provides a method of screening for an agent or a candidate compound capable of modulating the expression or activity/function of F-spondin. In one embodiment, the method comprises:

(a) contacting the F-spondin molecule, or a cell containing F-spondin, with an agent or a candidate compound, wherein said F-spondin molecule is:
(i) a DNA corresponding to any one of SEQ ID NOs: 1, 3 or 5;
(ii) a protein comprising any one of SEQ ID NOs: 2, 4 or 6;
(iii) a nucleic acid comprising a sequence hybridizable to any one of SEQ ID NOs: 1, 3 or 5, or a complement thereof under conditions of high stringency, or a protein comprising a sequence encoded by said hybridizable sequence; or
(iv) a nucleic acid at least 90% homologous to any one of SEQ ID NOs: 1, 3 or 5, or a complement thereof as determined using an NBLAST algorithm or a protein encoded thereby;

(b) determining whether or not the agent or candidate compound modulates the expression or activity/function of the F-spondin molecule;

wherein an agent or a candidate compound that increases the expression or activity/function of the F-spondin molecule is considered to be an agonist of F-spondin, and wherein an agent or a candidate compound that decreases the expression or activity/function of the F-spondin molecule is considered to be an antagonist of F-spondin.

In another particular embodiment, the method further comprises:

(d) measuring the effect of the agent or candidate compound on the level of expression or activity/function of at least one gene or gene product, which is a member of the PGE2, TGF-β or αvβ3 pathways.

In another particular embodiment, the member of the PGE2, TGF-β or αvβ3 pathways is selected from the group consisting of COL2A, aggrecan, MMP-13, BMP2, PGE2, MMP-1, TNF-α, and TGF-β1; and an agent or a candidate compound is identified as an agonist of F-spondin if the candidate compound increases the expression or activity/function of one or more of the molecules selected from the group consisting of COL2A, aggrecan, MMP-13, BMP2 and PGE2; and/or decreases the expression or activity/function of one or more of the molecules selected from the group consisting of MMP-1 and TNF-α; and/or activates latent TGF-β1; and wherein an agent or a candidate compound is identified as an antagonist of F-spondin if the agent or candidate compound decreases the expression or activity/function of one or more of the molecules selected from the group consisting of COL2A, aggrecan, MMP-13, BMP2 and PGE2; and/or increases the expression or activity/function of one or more of the molecules selected from the group consisting of MMP-1 and TNF-α; and/or prevents or inhibits activation of latent TGF-β1.

In another particular embodiment, the candidate compound is further tested for an effect in an animal model for arthritis, or a cartilage degenerative condition, wherein said arthritis or cartilage degenerative condition is characterized by elevated levels of F-spondin. Examples of animal models for studying arthritis and cartilage degenerative conditions may be found in the following publications, which are incorporated in their entireties: Ameye, L. G. et al., Current Opinion in Rheumatology (2006), 18(5):537-547; Warskyj, M. and Hukins, D W, Br. J. of Rheumatology (1990), 29:219-221; Bolter, S. M. et al., Biorheology, (2006), 43(3-4):379-388; Carlson, C. S. et al., J. Bone Miner. Res. (1996), September; 11(9):1209-1217; Moreau, M. et al., J. Rheumatology (2006), June; 33(6):1176-1183; Laurent, D. et al. Skeletal Radiol., (2006), August; 35(8):555-564; Hotta, H. et al. J. Orthop. Sci. (2005), November; 10(6):595-607; Mastbergen, S. C. et al. Rheumatology (Oxford), (2006), April; 45(4):405-413; and Mastbergen, S. C. et al., Osteoarthritis Cartilage (2006), January; 14(1):39-46. Chambers M. G. et al., Arthritis and Rheumatism (2001) June 44(6):1455-1465. In another particular embodiment, the potential inducers of F-spondin in cartilage/chondrocytes for use in a therapeutic setting may be selected from the group consisting of prostaglandin E2 (PGE2), cAMP inducers, Bone morphogenic protein 2 (BMP-2), Insulin-like growth factor (IGF), Fibroblast growth factor basic (FGFbasic) and Transforming Growth factor b1 (TGF-b1). These agents induced F-spondin expression in human chondrocytes as analyzed by TaqMan quantitative PCR. Accordingly, it is envisioned that these agents may be used for treating conditions as described herein.

In another particular embodiment, the determining expression or activity/function is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assays), immunohistochemistry, a Western blot, a dotblot analysis, a protein microarray, a flow cytometric technique and proteomics analysis.

In a further aspect, the invention provides uses of F-spondin, active fragments thereof, or modulators thereof including agents which modulate the expression or activity of F-spondin, in stimulating chondrocyte maturation and enhancing cartilage repair or in preventing or treating cartilage degeneration, including arthritic conditions.

In accordance with the present invention, a method for modulating chondrogenesis and cartilage degenerative disease is provided comprising modulating the expression or activity of F-spondin. In a particular such aspect, the maturation or differential growth of cartilage or chondrocytes is enhanced by modulation of F-spondin. The invention provides a method for producing cartilage at a cartilage defect site or of preventing or reducing cartilage degeneration including in an arthritic condition, comprising administering, including at the defect site, F-spondin, an active fragment thereof, or a modulating agent, such that the production or maturation of cartilage is stimulated or the degeneration of cartilage is affected.

In accordance with the present invention, a method for modulating bone formation or growth, including endochondral bone formation, or for modulating a bone disease, bone disorder, or bone degenerative disease is provided comprising modulating the expression or activity of F-spondin. In a particular such aspect, the differential growth of bone is enhanced by modulation, particularly by inhibition or blocking of F-spondin. The invention provides a method for producing bone at a defect site or fracture site or growth site or of preventing or reducing bone degeneration, comprising administering, including at the defect, fracture or growth site, F-spondin, an active fragment thereof, or a modulating agent, such that the production, formation or generation of bone is stimulated or the degeneration of bone is affected.

Other objects and advantages will become apparent to those skilled in the art from a review of the following description which proceeds with reference to the following illustrative drawings.

### BRIEF DESCRIPTION OF THE DRAWINGS

FIGS. 1A and 1B: Differential expression of F-spondin in normal and OA cartilage:

1A. RNA was extracted from 20 normal and 50 OA cartilage samples and pooled (each pool represents 10 individual cartilage samples). Affymetrix microarray was carried out.

1B. expression of F-spondin from normalized microarray data that is represented by arbitrary units.

FIG. 1C: Confirmation of differential expression of F-spondin in 10 normal and 18 OA cartilages by QPCR. Insert represents the average of normal and OA cartilage F-spondin expression with statistical significance (p<0.03).

FIG. 2: Western analysis of F-spondin in Normal and OA cartilage extracts: Fifty μg of total protein from normal and OA cartilage was resolved on 10% SDS-PAGE and F-spondin was immunodetected using rabbit anti-F-spondin polyclonal antibody. F-spondin was detected at ˜105 kDa.

FIG. 3: Immunodetection of F-spondin in OA cartilage: The expression of F-spondin was also confirmed by immunohistochemistry in lesional and non-lesional OA cartilage obtained at the time of surgery. Immunostaining demonstrates intense staining of F-spondin in superficial zone associated with chondrocytes and matrix. In non-lesional cartilage, immunostaining of F-spondin was similar but also observed in the middle zone. The F-spondin distribution was comparable to type II collagen in non-lesional cartilage.

FIG. 4: Microarray analysis of F-spondin expression in surgical model of OA.

FIGS. 5A and 5B: Distribution of F-spondin in the chick embryo growth plate FIG. 5A: Immunolocalization of NOS isoforms in the chick growth plate counterstained with Alcian blue. (A and B) Control section (incubated with pre-immune serum; (C and D) eNOS; (E and F) nNOS; (G and H) iNOS. In each case, the upper image is representative of the proliferative zone, while the lower image is from the hypertrophic region of the growth plate. Arrows indicate the presence of positively stained hypertrophic chondrocytes that border the vascular channels. A high level of eNOS and iNOS protein is also expressed by osteoblasts (arrow head). Magnifications 400×. FIG. 5B: Sections were immunostained using an antibody against F-spondin (B). Control sample (A) was incubated with pre-immune serum. Both sections were counterstained with alcian blue and photographed. Brown color indicates the presence of chondrocytes positive for F-spondin.

FIG. 6: Chondrogenesis of postnatal MSCs following exposure to TGF-β and BMP-2. Bone marrow-derived MSCs were modified to express 50 ng/ml BMP-2 or TGF-β1 using first generation recombinant adenoviral vectors and seeded into high density aggregates. After 21 d, aggregates were fixed, sectioned and stained for the presence of chondrogenic markers using toluidine blue (proteoglycan) and immunostaining for type II and X collagen. This pattern was consistent over three experiments.

FIG. 7: IL-1 inhibited F-spondin and aggrecan expression in OA cartilage. OA cartilage was stimulated with IL-1 (1 ng/ml) for 24-72 h and total RNA was isolated and QPCR was performed.

FIG. 8: Growth factors induce F-spondin expression in Human OA chondrocytes. The cells were adapted to serum free medium conditions for 24 h before treating with either Retinol (100 nM), TGF-β1 (2 ng/ml), FGF basic (25 ng/ml), FGF-18 (100 ng/ml) for 24 h and the cells were harvested for RNA isolation. All the growth factors induced F-spondin expression in chondrocytes.

FIGS. 9A and B: A: F-spondin deletion constructs with different domains: The F-spondin gene consists of various domains depicting an N-terminal signal peptide (SP), a reelin domain, spondin domain and a sequence of six thrombospondin domains in tandem. FS1: full length F-spondin; FS2, FS3, FS4 with one, three and five thrombospondin motifs; FS5: only the reelin domain; FS6: reelin and spondin domain with no thrombospondin motif and FS7: only six thrombospondin motifs. Mindin is a similar family member of F-spondin without a reelin domain and only one thrombospondin motif. B: Plasmin cleaves the C-terminus at two points, generating a soluble, 95 kDa, 656 amino acid F-spondin that contains all but TSP repeats number 5 and 6. The protease appears to cleave F-spondin between the FS segment. These fragments may then be used for diagnostic purposes.

FIG. 10: Transgene expression of F-spondin in human chondrocyte cell line C28I2 induced anabolic genes: Human chondrocyte cell line C28I2 was transfected by nucleofector reagent (Amaxa) with various F-spondin constructs (FS1: full length F-spondin, FS6: FS 7: and Min: Mindin) and the cells were harvested 24 h post transfection for RNA extraction and PCR.

FIG. 11: Blocking of F-spondin function by antibody: Human chondrocytes were grown in monolayer culture. The cells were adapted to serum free medium conditions for 24 h before treating with either F-spondin transfected supernatant (150 uL), LM609 (αvβ3) or R1 (F-spondin blocking antibody) for 24 h and the culture supernatant was collected for PGE2 estimation by RIA. The data is representative of one of the three experiments. F-spondin induced production of PGE2 was inhibited by R1 (TSR 3-6 blocking antibody). Similarly, blocking αvβ3 by LM609 also inhibited F-spondin induced PGE2 production.

FIG. 12: Activation of Latent TGF-β1 by exogenous addition of F-spondin in OA cartilage explant cultures: Human OA cartilage was grown as explant cultures in serum free Ham'F-12 medium. All the conditions were done in triplicate. To the cartilage explants recombinant human F-spondin (1 ug/ml) or IL-1 (1 ng/ml) were added and supernatants were collected after 24 h. Both active and total TGF-β1 was estimated. Addition of F-spondin increased the levels of active TGF-β1 observed in explants cultures without significant increase in total latent TGF-β1 secretion.

FIG. 13. Hypothetical actions of F-spondin and its fragments on chondrocyte functions: From the above preliminary studies, we hypothesize that F-spondin and its fragments activate both anabolic and catabolic effects. The catabolic effects of F-spondin may be activated via induction of PGE2/NURR1/cAMP pathway, whereas the anabolic effects may be via activation of latent TGF-β1. These actions of F-spondin are partly via ligation of integrin αvβ3, but may involve previously unidentified receptors.

FIG. 14. Chick chondrocytes express F-spondin as well as other growth plate maturation genes following RA treatment. Chick chondrocytes were stimulated with increasing doses of RA (10-100 nm) and harvested for gene expression analysis by qPCR after 5 days. The relative gene expression of type X collagen (ColX), alkaline phosphatae (AP), MMP-13, and F-spondin were assessed with increasing maturation on RA stimulation.

FIG. 15. F-spondin overexpression induces expression of chondrocyte maturation genes, AP and MMP-13, following RA treatment. Chick chondrocytes were transfected with either F-spondin or vector control (pcDNA3) and stimulated with RA at 100 nm for 5 days to induce maturation. *=p<0.05 vs pcDNA3.

FIG. 16. Inhibition of F-spondin decreases AP activity in RA stimulated cultures. Chick chondrocytes were treated with RA at 100 nm for 5 days with and without F-spondin antibodies and AP activity assessed. Antibodies with specificities to the spondin domain (medium grey) and TSR domain (black) inhibited AP activity compared to ctrl (noAb) (light grey). Note the greater inhibitory effect of the TSR domain antibody. * p=<0.05 versus no ab ctrl.

FIG. 17. The pro-maturation effect of F-spondin is not inhibited following neutralization of TGF-β activity by coculture with Latency Associated Peptide (LAP). AP activity was determined in chick chondrocyte cultures by ELISA following RA stimulation for 3 days. Cultures were either ctrl (RA only) or RA with either F-Spondin (1 ug/ml) or LAP (10 or 100 ng/ml), or RA with F-Spondin (1 ug/ml) and LAP (10 or 100 ng/ml) in combination.

FIG. 18. Blocking αvβ3 integrin inhibits the promaturation effect of F-spondin. Chondocyte cultures were transfected as previously and stimulated with RA for 3 days in the presence of IgG control, or αvβ3 blocking antibodies prior to assay for AP activity. Inhibition of αvβ3 led to a ˜50% suppression of F-spondin-induced AP activity but had no effect on baseline AP activity (vector ctrl).

FIGS. 19A and 19B. F-spondin is expressed in embryonic growth plate cartilage. (A) Immunohistochemical staining of F-spondin in embryonic chick tibia. Longitudinal sections from 18 d old chick embryos were stained with pre-immune serum (control—left panel) or F-spondin antibody R1 (f-spondin—right panel), and counterstained with alcian blue. The different regions of the growth plate representing different stages of chondrocyte maturation are indicated. Brown color shows the presence of chondrocytes and ECM positive for F-spondin. (B) Relative gene expression of F-spondin and chondrocyte maturation markers in chick tibial growth plate cartilage. Embryonic 18-day old chick tibias were separated into proliferative (P), hypertrophic (H) and calcified (C) regions by microdissection. RNA was extracted and analyzed for gene expression levels by semi-quantitative RT-PCR. Results are show as percentage change in mRNA levels from proliferative region.

FIGS. 20A, 20B and 20C. F-spondin regulates bone growth and morphology in mouse tibial organ cultures. Longitudinal growth and histological analysis of embryonic mouse tibia after 7 days of culture ex vivo. Tibia were treated with recombinant F-spondin FS (1 mg/ml) or an anti-F-spondin, TSR-domain specific (R1) antibody. (A) Representative images of whole tibia stained with alcian blue (proteoglycans) and alizarin red (mineral). (B) Tibial growth was measured after 7 days of organ culture and expressed as percent of original length at day 0. Average growth for control cultures has been set to 100% (C) H&E stained sections of corresponding limbs after 7 days treatment. Arrows indicate hypertrophic chondrocytes in the diaphyseal growth plate.

FIGS. 21A and 21B. F-spondin is upregulated during maturation of chick sternal chondrocytes. Upper sternal chondrocytes were exposed to 0, 10, 35 or 100 nM retinoic acid (RA) for 5 days in culture. (A) Alkaline phosphatase staining indicating dose-dependent maturation of chick chondrocytes in response to RA. (B) Relative gene expression levels of chondrocyte maturation markers were determined by semi-quantitative RT-PCR of RNA harvested from parallel cultures. Values represent mean±std deviation for triplicate samples. All results, except from F-spondin expression level with RA 10 treatment, are statistically significant different from RA 0, p<0.05.

FIGS. 22A and 22B. F-spondin enhances RA-induced chondrocyte maturation of embryonic chick chondrocytes. Upper sternal chondrocytes were transfected with full length F-spondin cDNA (FS1), or pcDNA3 vector control and treated with or without RA (100 nM). (A) Mineral accumulation by Von Kossa staining of transfected cultures following 7 d treatment with RA in b-glycerolphosphate containing medium. (B) Gene expression levels in transfected chondrocytes after 5 days in culture determined by semi-quantitative RT-PCR. Values are expressed as relative mRNA levels in comparison to pcDNA transfected control cells (=1). *=p<0.05 versus vector ctrl.

FIGS. 23A, 23B and 23C. F-spondin regulation of chondrocyte maturation requires the TSR domain. (A) Schematic representation of F-spondin protein domains and plasmid constructs encoding either full length (FS1) or TSR domain truncated (FS6) cDNAs. (B) Relative AP activity of FS or pcDNA3 control transfected cultures after 5 days treatment. (C) Relative AP activity following inhibition of F-spondin protein domains. Upper sternal chondrocytes were stimulated with 0, 10, 35 or 100 nM retinoic acid (RA) for 5 days in culture. Before RA treatment, endogenous F-spondin was inhibited by incubating cells with anti-FS antibodies with specificities to either the spondin domain (R4) or TSR domain (R1). Values represent mean standard deviation for triplicate samples. * Statistically significant different from control at the same RA concentration; p<0.05.

FIGS. 24A and 24B. TGF-β enhances chondrocyte maturation and is required for F-spondin mediated induction of AP. (A) Relative gene expression of chondrocyte maturation markers following TGF-β treatment. Upper sternal chondrocytes were treated with TGF-β at the indicated doses for 5 days and mRNA levels determined by quantitative PCR. Values are expressed relative to untreated controls. (B) Effect of TGF-β depletion on F-spondin-mediated induction of AP. The conditioned media of FS1 or pcDNA3 transfected chondrocytes was harvested and immunodepleted of TGF-β or left untreated, and added to separate cultures in the presence of RA. Graph shows relative AP gene expression after 24 h treatment. Values represent mean expression levels of triplicate samples relative to untreated supernatants from vector ctrl cultures (=1).

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The terms used herein have the meanings recognized and known to those of skill in the art, however, for convenience and completeness, particular terms and their meanings are set forth below.

The terms “patient” and “subject” mean all animals including humans. Examples of patients or subjects include humans, non-human primates, cows, dogs, cats, goats, sheep, pigs and any other domestic or non-domestic animals.

A “cartilage degenerative condition characterized by an increase in the level of expression of F-spondin”, as used herein, refers to a condition that presents itself in a patient with one or more of the symptoms associated with osteoarthritis or other cartilage degenerative conditions, such as joint stiffness, swelling or pain, while at the same time, exhibiting increased expression of the F-spondin gene or gene product, in one or more tissue samples from the subject, as described herein.

A “person prone to developing, or at risk for developing, a disease associated with an increase in the expression and/or activity/function of F-spondin”, as used herein, refers to a patient having a susceptibility to developing one or more conditions associated with an increase in the level of expression and/or activity/function of F-spondin in articular cartilage or in synovial fluid, as described herein, more particularly OA or other cartilage degenerative conditions, due to a genetic predisposition or due to overuse of a joint or damage or injury to a joint, or to any mixture of agents or risk factors for acquiring or developing OA or other cartilage degenerative conditions. An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of OA or a cartilage degenerative condition. An individual having one or more of these risk factors has a higher probability of developing OA or a cartilage degenerative condition than an individual without these risk factors.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted. In the present invention, “treatment” or “treating” refers to the amelioration of one or more symptoms or sequelae of arthritis, or a fibrosing disorder, including, but not limited to scleroderma, pulmonary fibrosis and retroperitoneal fibrosis. More particularly, treating refers to alleviating the symptoms of osteoarthritis, or other cartilage degenerative conditions, including but not limited to swelling or stiffness in one or more joints, or the pain associated with the swelling or stiffness. Most preferably, the treating is for the purpose of reducing or diminishing one or more symptoms or progression of a disease or disorder including any form of arthritis, particularly OA, as well as, other cartilage degenerative conditions or fibrosing disorders. Furthermore, in treating a subject, a medication useful for treating the conditions described herein may be administered to a subject already suffering from OA, as well as, other cartilage degenerative conditions, or to prevent or inhibit the occurrence of such condition or to slow or halt its progression.

A “biomarker” as used herein, refers to a specific molecule, the existence and levels of which are causally connected to a biological process, and reliably captures the state of said process. In the matter of the present invention, the nucleic acid of any one of SEQ ID NOs: 1, 3 or 5, (human, rat and mouse nucleic acid, respectively, which encode F-spondin) or the proteins of any one of SEQ ID NOs: 2, 4 or 6, (human, rat and mouse F-spondin protein, respectively) are envisioned for use in detecting osteoarthritis or other cartilage degenerative conditions or related conditions, or for use in predicting whether a subject may be predisposed to such diseases or conditions.

An “antibody” is any immunoglobulin, including antibodies and fragments thereof, that binds a specific epitope. Such an antibody that binds a specific epitope is said to be “immunospecific”. The term encompasses “polyclonal”, “monoclonal”, and “chimeric” antibodies, the last mentioned described in further detail in U.S. Pat. Nos. 4,816,397 and 4,816,567. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The “chimeric antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be a single chain antibody. (See, e.g., Curiel et al., U.S. Pat. No. 5,910,486 and U.S. Pat. No. 6,028,059). The antibody may be prepared in, but not limited to, mice, rats, rabbits, goats, sheep, swine, dogs, cats, or horses. As used herein, the term “single-chain antibody” refers to a polypeptide comprising a V_{H} region and a V_{L} region in polypeptide linkage, generally linked via a spacer peptide (e.g., [Gly-Gly-Gly-Gly-Ser]ₓ), and which may comprise additional amino acid sequences at the amino- and/or carboxy-termini. For example, a single-chain antibody may comprise a tether segment for linking to the encoding polynucleotide. As an example, a scFv (single chain fragment variable) is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope). In the present invention, antibodies of particular relevance include anti-spondin antibodies commercially available from GenWay (15-288-22651), which is a chicken anti-spondin antibody; from Novus Biologicals (H00010418-M01), which is a mouse anti-human F-spondin clone 3F4; and GeneTex (GTX14271), which is a chicken anti-spondin 1 antibody.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide. Exemplary protein or polypeptide fragments of F-spondin that may be used for diagnostic purposes include those shown in Table 2, although smaller fragments obtained from SEQ ID NOs: 2, 4, 6, or any one of SEQ ID NOs: 34-40 may be used (SEQ ID NOs: 34-39 correspond to thrombospondin repeats 1-6, respectively, and SEQ ID NO: 40 is the signal peptide). Included in this are possible fragments that may be motifs for latent TGF-beta binding and activation. These are found at residues 448-451 of SEQ ID NO: 2; residues 620-623 of SEQ ID NO: 2; residues 674-677 of SEQ ID NO: 2 and residues 783-786 of SEQ ID NO: 2. Fragments corresponding to residues 682-685 and 729-732 of SEQ ID NO: 2 may also be relevant for diagnostic use. Since possible protease cleavage sites may lie between residues 415-446 of SEQ ID NO: 2, it is envisioned that any peptide fragment resulting from such cleavage may be useful for diagnostic purposes.

A “small molecule” or “small organic molecule” is an organic compound (or organic compound complexed with an inorganic compound (e.g., metal)) that has a molecular weight of less than 3 kilodaltons, and preferably less than 1.5 kilodaltons. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, and preferably less than 1.5 kilodaltons, and more preferably less than about 1 kilodalton.

“Gene Product” as used herein, unless otherwise indicated, is a protein or polypeptide encoded by the nucleic acid sequence identified by the methods of the present invention, including but not limited to any one of SEQ ID NOs: 1, 3 or 5; a nucleic acid comprising a sequence hybridizable to any one of SEQ ID NOs: 1, 3 or 5, or its complement under conditions of high stringency, or a protein comprising a sequence encoded by said hybridizable sequence (SEQ ID NOs: 2, 4 or 6, respectively); a nucleic acid at least 90% homologous to any one of SEQ ID NOs: 1, 3 or 5, its complement as determined using, for example, the NBLAST algorithm; a nucleic acid at least 90% homologous to any one of SEQ ID NOs: 1, 3 or 5, or a fragment or derivative of any of the foregoing proteins or nucleic acids.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA. The oligonucleotides of the invention are preferably from 10 to 50 nucleotides in length, even more preferably from 20-30 nucleotides in length or from 15-25 nucleotides in length, and may be DNA, RNA or synthetic nucleic acid, and may be chemically or biochemically modified or may contain non-natural or derivatized nucleotide bases, as will be appreciated by those skilled in the art. Also included are synthetic molecules that mimic polynucleotides in their ability to bind to a designated sequence to form a stable hybrid. Such molecules are known in the art and include, for example, peptide nucleic acids (PNAs) in which peptide linkages substitute for phosphate linkages in the backbone of the molecule.

A labeled oligonucleotide or primer may be utilized in the methods, assays and kits of the present invention. The labeled oligonucleotide may be utilized as a primer in PCR or other method of amplification and may be utilized in analysis, as a reactor or binding partner of the resulting amplified product. In certain methods, where sufficient concentration or sequestration of the nucleic acid has occurred, and wherein the oligonucleotide label and methods utilized are appropriately and sufficiently sensitive, the nucleic acid may be directly analyzed, with the presence of, or presence of a particular label indicative of the result and diagnostic of a cartilage degenerative condition. After the labeled oligonucleotide or primer has had an opportunity to react with sites within the sample, the resulting product may be examined by known techniques, which may vary with the nature of the label attached. The label utilized may be radioactive or non-radioactive, including fluorescent, colorimetric or enzymatic. In addition, the label may be, for instance, a physical or antigenic tag which is characterized by its activity or binding.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence according to the invention. Pairs of single-stranded DNA primers can be annealed to sequences within a target nucleic acid. One probe that has proven useful in the present invention was obtained from Applied Biosystems as Catalog Number Hs00391824 ml.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

As used herein, “conditions of high stringency” refer to procedures that utilize the following conditions: Prehybridization of filters containing DNA is carried out for 15 minutes to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5−20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

“Operably linked” when describing the relationship between two polynucleotide sequences, means that they are functionally linked to each other. For example, a promoter is operably linked to a coding sequence if it controls the transcription of the sequence. As a regulatory sequence commonly used promoter elements as well as enhancers may be used. Generally, such expression regulation sequences are derived from genes that are expressed primarily in the tissue or cell type chosen. Preferably, the genes from which these expression regulation sequences are obtained are expressed substantially only in the tissue or cell type chosen, although secondary expression in other tissue and/or cell types is acceptable if expression of the recombinant DNA in the transgene in such tissue or cell type is not detrimental to the transgenic animal.

An “amplicon” is a nucleic acid sequence amplified by the specific primers during the course of a polymerase chain reaction (PCR), i.e., the fragment produced by PCR amplification using a primer pair of the present invention.

As used herein, “amplifying” refers to the generation of additional copies of a nucleic acid sequence. A variety of methods have been developed to amplify nucleic acid sequences, including the polymerase chain reaction (PCR). PCR amplification of a nucleic acid sequence generally results in the exponential amplification of a nucleic acid sequence(s) and or fragments thereof.

“Complementary” or a “complement” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA.

A “reporter gene” or “reporter molecule” refers to a gene whose phenotypic expression is easy to monitor and is used to study promoter activity in different tissues or developmental stages. Recombinant DNA constructs are made in which the reporter gene is attached to a promoter region of particular interest and the construct transfected into a cell or organism. As used herein, a “reporter” gene is a nucleic acid that is readily detectable and/or encodes a gene product that is readily detectable such as green fluorescent protein (as described in U.S. Pat. No. 5,625,048 issued Apr. 29, 1997, and WO 97/26333, published Jul. 24, 1997, the disclosures of each are hereby incorporated by reference herein in their entireties), or red fluorescent protein, or yellow fluorescent protein, or wheat germ agglutinin (WGA) or a WGA-type molecule or luciferase.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to selectively hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to selectively hybridize therewith and thereby form the template for the synthesis of the extension product.

By “homologous” is meant a same sense nucleic acid which possesses a level of similarity with the target nucleic acid within reason and within standards known and accepted in the art. With regard to PCR, the term “homologous” may be used to refer to an amplicon that exhibits a high level of nucleic acid similarity to another nucleic acid, e.g., the template cDNA. As is understood in the art, enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplified nucleic acid (i.e., amplicon) need not be completely identical in every nucleotide to the template nucleic acid.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Two amino acid sequences are “substantially homologous” when at least about 70% of the amino acid residues (preferably at least about 80%, and most preferably at least about 90 or 95%) are identical, or represent conservative substitutions.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350. The PCR methods of the invention include standard PCR, reverse transcriptase PCR (RT-PCR), real-time PCR and quantitative PCR, each of which are procedures known to those skilled in the art.

In certain embodiments, a method of the invention comprises detecting the presence of F-spondin nucleic acid, such as an mRNA, in a sample. Optionally, the method involves obtaining a quantitative measure of the F-spondin expressed nucleic acid in the sample. In view of this specification, one of skill in the art will recognize a wide range of techniques that may be employed to detect and optionally quantitate the presence of a nucleic acid. Nucleic acid detection systems generally involve preparing a purified nucleic acid fraction of a sample, and subjecting the sample to a direct detection assay or an amplification process followed by a detection assay. Amplification may be achieved, for example, by polymerase chain reaction (PCR), reverse transcriptase (RT) and coupled RT-PCR. Detection of a nucleic acid is generally accomplished by probing the purified nucleic acid fraction with a probe that hybridizes to the nucleic acid of interest, and in many instances detection involves an amplification as well. Northern blots, dot blots, microarrays, quantitative PCR, real-time PCR and quantitative RT-PCR are all well known methods for detecting a nucleic acid in a sample.

As used herein “arrays” or “microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522. Arrays or microarrays are commonly referred to as “DNA chips”. As used herein, arrays/microarrays may be interchangeably referred to as detection reagents or kits.

“Modulation” or “modulates” or “modulating” refers to up regulation (eg, activation or stimulation), or down regulation (eg, inhibition or suppression) of a response, or the two in combination or apart. In the manner of the present invention, modulation or modulating refers to either stimulation of expression or activity/function of F-spondin or suppression of expression or activity/function of F-spondin.

As used herein, the term “candidate compound” or “candidate therapeutic” or “test compound” or “test agent” refers to any compound or molecule that is to be tested. As used herein, the terms, which are used interchangeably, refer to biological or chemical compounds such as simple or complex organic or inorganic molecules, peptides, proteins, antibodies, oligonucleotides, polynucleotides, carbohydrates, or lipoproteins. A vast array of compounds can be synthesized, for example oligomers, such as oligopeptides and oligonucleotides, and synthetic organic compounds based on various core structures, and these are also included in the terms noted above. In addition, various natural sources can provide compounds for screening, such as plant or animal extracts, and the like. Compounds can be tested singly or in combination with one another. Candidate compounds can be randomly selected or rationally selected or designed. As used herein, a candidate compound is said to be “randomly selected” when the compound is chosen randomly without considering the specific interaction between the compound and the target site. As used herein, a candidate compound is said to be “rationally selected or designed”, when the compound is chosen on a nonrandom basis which takes into account the specific interaction between the compound and the target site and/or the conformation in connection with the compound's action. Moreover, the compound may be selected by its effect on the gene expression profile obtained from screening in vitro or in vivo. For example, the gene expression data for chondrocytes can be accessed online through databases including Pub Med, Human Genome Project (HGP), Gene Bank and PDB (Protein Data Bank).

By “effectiveness of therapy” is meant that upon treating a subject with an agent that modulates F-spondin expression or activity/function, one can determine whether the treatment has resulted in the desired outcome. For example, in the case of treating a patient having levels of F-spondin that are outside of the range that one might observe in a normal, non-arthritic subject, with an agent that either increases or decreases expression or activity/function of F-spondin, one may observe a change in one or more symptoms associated with the medical condition being treated (eg. the swelling, stiffness or pain associated with arthritis).

“Peripheral neuropathy” is failure of the nerves that carry information to and from the brain and spinal cord. This produces symptoms like pain, loss of sensation, and inability to control muscles. In some cases, failure of nerves controlling blood vessels, intestinal function, and other organs results in abnormal blood pressure, digestion, and loss of other basic involuntary processes. Peripheral neuropathy may involve damage to a single nerve or nerve group mononeuropathy or may affect multiple nerves (polyneuropathy). Risk factors for neuropathy include diabetes, heavy alcohol use, and exposure to certain chemicals and drugs. Some people have a hereditary predisposition for neuropathy. Prolonged pressure on a nerve is another risk for developing a nerve injury. Pressure injury may be caused by prolonged immobility (such as a long surgical procedure or lengthy illness) or compression of a nerve by casts, splints, braces, crutches, or other devices.

The phrase “neuromuscular disorders that put abnormal stress on a joint” refers to any type of medical condition in which there is damage to a nerve that results in partial or total loss of muscle control, which over time results in an undue stress to the joints. Such medical conditions include, but are not limited to, for example, muscular dystrophy, amyotrophic lateral sclerosis (ALS), post polio syndrome, multiple sclerosis, Parkinson's disease, spinal muscular atrophy, and the like.

“Cartilage Degenerative Condition” refers to any condition which results in the breakdown of cartilage in joints, thus resulting in pain, stiffness and sometimes swelling and inflammation in the affected area. Examples of such cartilage degenerative conditions include, but are not limited to, osteoarthritis, rheumatoid arthritis, gouty arthritis, psoriatic arthritis, chondrosarcomas, to name a few.

“Osteoarthritis”, or “OA”, which is the most common form of arthritis, is a complex disease whose etiology is unknown. Evidence is growing for the role of systemic factors, including, but not limited to, genetics, dietary intake, estrogen use, and bone density, as well as local biomechanical factors, including but not limited to, muscle weakness, obesity, and joint laxity. Injury, fractures around a joint surface, and overuse factors are also frequently involved in the development of osteoarthritis. These “risk factors for development of OA” are particularly important in weight-bearing joints, and modifying them may present opportunities for prevention of osteoarthritis-related pain and disability. Osteoarthritis may occur secondary to an injury to the joint due to a fracture, repetitive or overuse injury, or metabolic disorders (e.g., hyperparathyroidism). Additionally, gout and other forms of crystalline joint disease may lead to OA of a joint. Obesity, or being overweight, is a risk factor for knee osteoarthritis more commonly in females; this is less commonly seen in the hip joint. Recreational running does not increase the incidence of OA, but participation in competitive contact sports does. Specifically, impact sports that repetitively load a joint increase the injury to a joint. If cartilage in a joint is injured, it cannot regenerate, and the new forces that are created are abnormal, leading to further stresses, and the cycle may propagate. “Osteoarthritis” is also referred to as “degenerative arthritis” and is a disease that causes the breakdown of the cartilage in joints. Normally, cartilage acts as a smooth, cushioning material inside joints. In osteoarthritis, the cartilage becomes rough and flaky, and small pieces break off. The bone surface of the joint also becomes rough and irregular. As a result, movement of the joint becomes painful and difficult. Osteoarthritis occurs most often in weight-bearing joints, such as the neck, lower back, knees and hips. It also often affects the fingers. Osteoarthritis (OA) is thus a degeneration or ‘wear and tear’ of articular (joint surface) cartilage usually accompanied by an overgrowth of bone (osteophytes), narrowing of the joint space, sclerosis or hardening of bone at the joint surface, and deformity in joints. OA is not usually associated with inflammation, although swelling of the joint does frequently occur in OA. Osteoarthritis, is sometimes referred to as degenerative joint disease, DJD. Other forms of arthritis (rheumatoid, post-traumatic, and other inflammatory disorders) frequently may have OA as the end-stage, making differentiation difficult.

“Rheumatoid” and “juvenile rheumatoid” (affecting young people) arthritis (RA) are serious, painful joint diseases. RA primarily affects the cartilage and tissues that surround the lubricating fluid in the joint. The tissues in and around the joint are often degenerated or completely destroyed and replaced with scar tissue. RA can affect the entire body, but it most often affects the small joints of the fingers and hands. These joints become swollen, tender, and in advanced cases, deformed. The pain and deformity of advanced RA is often crippling. Rheumatoid arthritis affects over two million Americans. It occurs in women twice as frequently as men, often in people aged under 40 years old. Juvenile RA, as the name states, can involve even young children. The primary causes (onset factors) of rheumatoid arthritis appear to be linked to bacterial infections, nutritional deficiencies, or physical and/or emotional stress.

“Gouty” arthritis occurs mainly in people who are ‘living the high life’ eating rich foods, red meats, and regularly drinking alcohol. It is caused by the formation of uric acid crystals in the bloodstream (another chemistry imbalance), which find their way into the joints and their surrounding tissues, causing extremely sharp, needle-like pain in the joints (especially the joints of the big toe). Fever, body chills, sweats, and loss of joint motion often accompany this intense pain. Over 90% of gout sufferers are overweight males, over the age of forty. Health problems related to or caused by gout include indigestion, constipation, depression, headache, a higher risk of heart and kidney disease, and various skin conditions.

“Psoriatic” arthritis is similar to rheumatoid arthritis. Psoriatic arthritis usually affects people with psoriasis of the skin, and/or nails (common symptoms include a characteristic red, flaky or scaly skin rash, and thick, eroded nails) or those with a family history of psoriasis. Psoriatic arthritis causes pain, inflammation, swelling, and eventually degeneration, primarily in the joints of the fingers and toes, and sometimes the hips and spine.

“Development” or “progression” of OA or a cartilage degenerative condition herein means initial manifestations and/or ensuing progression of the disorder. Development of OA or a cartilage degenerative condition can be detectable and assessed using standard clinical techniques, such as measurement of swelling or stiffness in one or more joints, or pain in the joint. However, development also refers to disease progression that may be undetectable. For purposes of this invention, development or progression refers to the biological course of the disease state. “Development” includes occurrence, recurrence, and onset. As used herein “onset” or “occurrence” of OA or a cartilage degenerative condition includes initial onset and/or recurrence.

“Screening”, “diagnosing” or prognosing” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Agonist” refers to an agent that mimics or up-regulates (e.g., potentiates or supplements) the bioactivity of a protein. An agonist may be a wild-type protein or derivative thereof having at least one bioactivity of the wild-type protein. An agonist may also be a compound that up-regulates expression of a gene or which increases at least one bioactivity of a protein. An agonist may also be a compound which increases the interaction of a polypeptide with another molecule, e.g., a target peptide or nucleic acid. An agonist may also be a compound that increases or up-regulates the activity and/or function of a protein, peptide, an enzyme or biofactor.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. An antagonist may also be a compound that decrease or down-regulate the activity and/or function of a protein, peptide, an enzyme or biofactor.

The term “cartilage” refers to a type of connective tissue that contains chondrocytes or chondrocyte-like cells (having many, but not all characteristics of chondrocytes) and intercellular material (e.g., Types I, II, IX and XI collagen), proteoglycans (e.g., chondroitin sulfate, keratan sulfate, and dermatan sulfate proteoglycans) and other proteins. Cartilage includes articular and non-articular cartilage.

“Articular cartilage”, also referred to as hyaline cartilage, refers to an avascular, non-mineralized connective tissue, which covers the articulating surfaces of bones in joints and serves as a friction reducing interface between two opposing bone surfaces. Articular cartilage allows movement in joints without direct bone-to-bone contact. Articular cartilage has no tendency to ossification. The cartilage surface appears smooth and pearly macroscopically, and is finely granular under high power magnification. Articular cartilage derives nutrients partly from the vessels of the neighboring synovial membrane and partly from the vessels of the bone it covers. Articular cartilage is associated with the presence of Type II and Type IX collagen and various well-characterized proteoglycans, and with the absence of Type X collagen, which is associated with endochondral bone formation. For a detailed description of articular cartilage microstructure, see, for example, Aydelotte and Kuettner, Conn. Tiss. Res., 18, p. 205 (1988); Zanetti et al., J. Cell Biol., 101, p. 53 (1985); and Poole et al., J. Anat., 138, p. 13 (1984).

“Non-articular cartilage” refers to cartilage that does not cover articulating surfaces and includes fibrocartilage (including interarticular fibrocartilage, fibrocartilaginous disc, connecting fibrocartilage and circumferential fibrocartilage) and elastic cartilage. In fibrocartilage, the micropolysaccharide network is interlaced with prominent collagen bundles, and the chondrocytes are more widely scattered than in hyaline or articular cartilage. Interarticular fibrocartilage is found in joints which are exposed to concussion and subject to frequent movement, e.g., the meniscus of the knee. Examples of such joints include but are not limited to the temporo-mandibular, sterno-clavicular, acromio-clavicular, wrist and knee joints. Secondary cartilaginous joints are formed by discs of fibrocartilage. Such fibrocartilaginous discs, which adhere closely to both of the opposed surfaces, are composed of concentric rings of fibrous tissue, with cartilaginous laminae interposed. An example of such fibrocartilaginous disc is the intervertebral disc of the spine. Connecting fibrocartilage is interposed between the bony surfaces of those joints, which allow for slight mobility as between the bodies of the vertebrae and between the pubic bones. Circumferential fibrocartilage surrounds the margin of some of the articular cavities, such as the cotyloid cavity of the hip and the glenoid cavity of the shoulder.

Elastic cartilage contains fibers of collagen that are histologically similar to elastin fibers. Such cartilage is found in the auricle of the external ear, the eustachian tubes, the cornicula laryngis and the epiglottis. As with all cartilage, elastic cartilage also contains chondrocytes and a matrix, the latter being pervaded in every direction, by a network of yellow elastic fibers, branching and anastomosing in all directions except immediately around each cell, where there is a variable amount of non-fibrillated, hyaline, intercellular substance.

The term “synovial fluid” refers to a thin, lubricating substance within the synovial cavity that reduces friction within the joint. Synovial fluid lubricates and facilitates movement of the joint. The term “synovium” refers to the thin layer of connective tissue with a free smooth surface that lines the capsule of a joint. The “synovial membrane” refers to the connective-tissue membrane that lines the cavity of a synovial joint and produces the synovial fluid.

The F-spondin gene, as disclosed herein, is expressed in arthritic tissues (e.g., cartilage in a human afflicted with osteoarthritis) in elevated amounts relative to, i.e., to a greater extent than in the corresponding tissues of humans who do not suffer from osteoarthritis. Messenger RNA transcribed from the gene, and protein translated from such mRNA, is present in arthritic tissues and/or synovial fluid associated with such tissues in an amount at least about one and a half (1.5) times, or at least about five (5) times, or at least ten (10) times and more preferably about ninety (90) fold greater than the levels of mRNA and protein found in corresponding tissues found in humans who do not suffer from osteoarthritis, as measured by quantitative PCR (QPCR).

“Aggrecan” is the shortened name of the large aggregating chondroitin sulphate proteoglycan. Aggrecan, which is one of the most widely studied proteoglycans, is abundant; it represents up to 10% of the dry weight of cartilage (articular cartilage is up to 75% water). Many individual monomers of aggrecan bind to hyaluronic acid to form an aggregate, it is the monomer which is termed aggrecan. These aggregates are comprised of up to 100 monomers attached to a single chain of hyaluronic acid (HA). One distinct property of aggrecan is its extreme content of negatively charged polysaccharide chains. This contributes in excess of 10,000 negative charges to aggrecan and a couple of orders of magnitude more to the aggregate creating an osmotic environment that is responsible for the extremely high osmotic swelling pressure of cartilage. This swelling pressure is counteracted by the resistance of the intact collagen fibres giving cartilage its characteristic properties of being able to resist compressive forces and having a high tensile strength.

“Matrix metalloproteinase-13” (“MMP-13”, also known as collagenase 3) and “Matrix metalloproteinase-1” (“MMP-1”, also known as Interstitial collagenase) are capable of degrading triple-helical fibrillar collagens into distinctive ¾ and ¼ fragments. These collagens are the major components of bone and cartilage, and MMPs are the only known mammalian enzymes capable of degrading them. The MMPs play an important role in tissue remodeling.

“Bone morphogenic protein 2” or “BMP-2” is a protein that induces the formation of bone and cartilage. Bone morphogenic protein 2 belongs to a superfamily called transforming growth factor beta (TGF-beta). The gene for BMP2 is on chromosome 20 in band 20p12.3. Three sets of variations within the BMP2 gene reportedly triple the risk of developing osteoporosis.

“Prostaglandin E2” or “PGE2” is a member of the prostaglandins, a group of hormone-like substances that participate in a wide range of body functions such as the contraction and relaxation of smooth muscle, the dilation and constriction of blood vessels, control of blood pressure, and modulation of inflammation. Prostaglandin E2 (PGE-2) is released by blood vessel walls in response to infection or inflammation that acts on the brain to induce fever. Thus, PGE2 is the ultimate mediator of the febrile response.

Transforming growth factor: (TGF) One of several proteins secreted by transformed cells that can stimulate the growth of normal cells. Transforming growth factor alpha (TGF alpha or TGF-α) binds the epidermal growth factor receptor (EGFR) and stimulates the growth of endothelial cells (cells that line the inside of blood vessels). “Transforming growth factor beta” (“TGF-beta”, or “TGF-b” or “TGF-β”) is found in hemotopoietic (blood-forming) tissue and initiates a signaling pathway that suppresses the early development of cancer cells. Transforming growth factor beta is synthesised in a wide variety of tissues including platelets, placenta, and both normal and transformed cell lines. It acts synergistically with TGF-alpha in inducing phenotypic transformation and can also act as a negative autocrine growth factor. TGF-β also has a potential role in embryonal development, cellular differentiation, hormone secretion, and immune function. There are at least three forms of TGF-β: TGFD-β1, TGF-β2, and TGF-β1.2. The latter is a heterodimer made up of both TGF-β1 and TGF-β2. “Transforming growth factor-beta 1” (TGF-β1) is a potent profibrotic cytokine, which might contribute to airway wall thickening and fibrosis of bronchiolar and alveolar submucosa.

“Interleukin-8” or “IL-8” is a proinflammatory cytokine structurally related to platelet factor 4, which is released by several cell types (eg, monocytes, macrophages, T cells, endothelial cells, tumor cells) in response to an inflammatory stimulus. It activates neutrophils and is a chemokine for neutrophils and T lymphocytes. It is also an angiogenic factor and induces hypertrophic changes in chondrocytes.

“Interleukin-1” or “IL-1” is a pro-inflammatory cytokine (17 kD: 152 amino acids) secreted by monocytes, macrophages or accessory cells and is involved in the activation of both T-lymphocytes and B-lymphocytes and potentiates their response to antigens or mitogens. Its biological effects include the ability to replace macrophage requirements for T-cell activation, as well as affecting a wide range of other cell types. at least two IL-1 genes are active and alpha and beta forms of IL-1 are recognised. It is released early in an immune system response by monocytes and macrophages. It stimulates T-cell proliferation and protein synthesis. Another effect of IL-1 is that it causes fever.

“Tumor necrosis factor alpha” (“TNFα”, “cachexin” or “cachectin”) is an important inflammatory cytokine that is involved in systemic inflammation and the acute phase response.

“Chondrocytes” are the only cells found in cartilage. They produce and maintain the cartilagenous matrix. From least- to terminally-differentiated, the chondrocytic lineage is:
1. Colony-forming unit-fibroblast (CFU-F)
2. Mesenchymal stem cell/marrow stromal cell (MSC)
3. Chondrocyte
4. Hypertrophic chondrocyte

When referring to bone or cartilage, mesenchymal stem cells (MSC) are commonly known as osteochondrogenic (or osteogenic, chondrogenic, osteoprogenitor, etc.) cells since a single MSC has shown the ability to differentiate into chondrocytes or osteoblasts, depending on the medium. In vivo, differentiation of a MSC in a vascularized area (such as bone) yields an osteoblast, whereas differentiation of a MSC in a non-vascularized area (such as cartilage) yields a chondrocyte. Chondrocytes undergo terminal differentiation when they become hypertrophic during endochondral ossification. This last stage is characterized by major phenotypic changes in the cell.

Thus, chondrocytes emerging in the limb or other locations during embryogenesis are currently considered terminally differentiated cells and thus represent the last stage of differentiation in the chondrogenic cell lineage. Most chondrocytes, however, undergo further major phenotypic changes during late embryogenesis and early postnatal life as they take part in the endochondral ossification process. During this process, “resting” chondrocytes first enter an active, proliferative phase and then develop into large, round “hypertrophic chondrocytes” with unique phenotypic traits. Mature hypertrophic chondrocytes secrete a collagen X-rich matrix and eventually undergo apoptosis to leave a cartilage scaffold that is mineralized before deposition of new bone (Hunziker E B, (1994), Mechanism of longitudinal bone growth and its regulation by growth plate chondrocytes. Microsc Res Tech 28:505-519) It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### GENERAL DESCRIPTION

In its broadest aspect, the present invention is based on the identification of enhanced levels of F-spondin in osteoarthritic cartilage and synovium, during chondrocyte maturation, and as a late stage marker for chondrocyte differentiation. Further, the identification of F-spondin in articular cartilage and synovial fluid from patients suffering from osteoarthritis (OA) suggests that F-spondin may be used as a biomarker for the screening, diagnosis or prognosis of patients suspected of having OA or other cartilage degenerative conditions. The recognition and identification of factor(s) that regulate chondrogenesis and chondrocyte maturation is of great importance from both a pathophysiological and a therapeutic standpoint. Thus, one purpose of this invention is to utilize F-spondin, including fragments thereof, agonists and antagonists, and modulators thereof, that are for normal cartilage development and progression of cartilage disorders, including arthritis, to further understand chondrogenesis and cartilage degeneration and to provide new molecular targets for prediction, diagnosis and treatment of cartilage-related diseases. OA chondrocytes have been shown to express markers associated with growth plate chondrocyte maturation, and the present invention is further based on the recognition that F-spondin is expressed in embryonic growth plate cartilage and enhances the expression of chondrocyte maturation markers. Thus, F-spondin may be used to stimulate and enhance chondrocyte maturation, particularly in conditions where further or enhanced maturation is desired, such as in cartilage degeneration, cartilage repair, including as an adjunct to cell repair therapies and as a preventative after cartilage injury. Accordingly, methods are proposed for determining the presence of OA or a cartilage degenerative condition in a subject, or for monitoring cartilage repair, or for assessing the risk of developing OA or a cartilage degenerative condition, or for determining a patient's response to therapies through use of F-spondin as a biomarker for these conditions. Based on these identifications, the present invention provides methods of detecting F-spondin as well as reagents needed to accomplish this task. The invention specifically provides nucleotide probes for detecting the F-spondin gene and antibodies for detecting the proteins encoded by this gene, and methods of detecting the F-spondin gene or gene product in a sample, methods of determining a risk of having or developing a disorder associated with the presence of the F-spondin gene, methods of screening for candidate compounds used to treat cartilage disorders associated with the presence of the F-spondin gene, methods of treating cartilage disorders associated with the presence of the F-spondin gene, and methods of using the probes and antibodies of the present invention for detection of OA or other related cartilage degenerative conditions.

The inventors have demonstrated that F-spondin regulates articular cartilage metabolism and is involved in chondrocyte maturation. Further, the present invention demonstrates that F-spondin plays a role in endochondral bone formation and growth. Using chondrocyte maturation models and bone culture systems, F-spondin is shown to be upregulated during chondrocyte maturation, is a late stage marker of chondrocyte terminal differentiation, and regulates bone growth, particularly endochondral bone growth. Modulation of F-spondin has application in both degenerative diseases of articular cartilage and tracheal cartilage or other permanent cartilage structures and in transient cartilage or at sites or under conditions wherein cartilage is resorbed and replaced with bone. Thus, in embryonic cartilaginous skeleton, the epiphyseal growth plates of long bones, the cartilaginous callus formed at fracture sites, and the tissue created during distraction osteogenesis F-spondin and modulation thereof may be utilized. F-spondin therefore has application in situations or under conditions wherein cartilage is replaced by bone or bone growth is warranted or necessary. Modulation of F-spondin may be used in the modulation, alleviation or treatment of diseases of the transient growth cartilage of the long bones, including chondrodysplasias and dwarfism, on in bone diseases, bone degeneration, bone fractures or bone cancer where replacement of bone or endochondral bone formation is warranted or helpful.

To identify genes and gene products involved in human osteoarthritis, the present inventors have analyzed the differences in gene expression in diseased cartilage (cartilage from patients suffering from OA) compared to cartilage from healthy patients without OA using microarray analysis, quantitative PCR and immunoblot analysis. The inventors have discovered that patients diagnosed as having OA have a significant increase in expression of F-spondin in chondrocytes compared to normal patients not suffering from OA and based on these findings, propose methods for diagnosing and prognosing patients suspected of having, or under treatment for, osteoarthritis and other cartilage degenerative conditions. Moreover, the data presented herein demonstrates that F-spondin is overexpressed in OA cartilage and induced by prostaglandin E2 in a cAMP dependent pathway. Furthermore, overexpression of F-spondin in a chondrocyte cell line induced anabolic gene expression such as aggrecan, type II collagen, BMP-2 and TGF-β1 and inhibited pro-inflammatory cytokines, including TNF-alpha expression. It has also been shown that F-spondin is expressed predominantly in hypertrophic and calcified zones of chicken growth plate and significantly induced in an osteoarthritis rat model. An additional notable and previously unrecognized function of F-spondin was shown to be its capacity to activate latent TGF-beta. As such, TGF-β1 activates anabolic activities of chondrocytes and may promote synthesis of extracellular matrix. Accordingly, one aspect of the invention provides for the use of F-spondin as a hypertrophy and mineralization biomarker of chondrocytes and may be used to follow the progression of disease. Since it activates anabolic activities, and decreases pro-inflammatory cytokines, therapies that modulate F-spondin expression and/or function may have application in the treatment of osteoarthritis and inflammatory arthritis. Furthermore, since F-spondin activates latent TGF-β, inhibitors of its activity could be useful for treating fibrosing disorders, including, but not limited to scleroderma, pulmonary fibrosis and retroperitoneal fibrosis.

More particularly, the invention relates to the correlation between the presence of F-spondin in cartilage and the onset or predisposition for cartilage degenerative conditions, for example, osteoarthritis. Further, it is an object of the invention to utilize the nucleic acid and/or protein sequences for the preparation of reagents for determining the presence of osteoarthritis in a subject or for assessing the risk of developing osteoarthritis or other cartilage degenerative conditions. Based on these identifications, the present invention provides methods of detecting these nucleic acids or proteins, as well as reagents needed to accomplish this task. The invention specifically provides nucleic acids encoding the F-spondin protein, or fragments thereof, and methods of identifying the presence and/or level of either the nucleic acid or the protein encoded by the nucleic acids, antibodies to the proteins encoded by the nucleic acids and methods of detecting these in a sample, methods of determining a risk of having or susceptibility for developing a disorder associated with the presence of such gene, methods of screening for compounds used to treat disorders associated with the presence of such gene, methods of treating disorders associated with the presence of such gene, and methods of using the sequences of the present invention for detection of osteoarthritis or other cartilage degenerative conditions, or for determining the susceptibility of a subject to developing osteoarthritis, or other cartilage degenerative conditions and the pain associated with these disorders. The present invention also provides nucleotide sequences and encoded amino acid sequences for F-spondin. More particularly, the nucleic acid sequence encoding human F-spondin is shown in SEQ ID NO: 1; the nucleic acid sequence encoding rat F-spondin is shown in SEQ ID NO: 3, and the nucleic acid encoding mouse F-spondin is shown in SEQ ID NO: 5. In one particular embodiment, the human F-spondin protein of SEQ ID NO: 2 is encoded by a nucleic acid comprising the DNA sequence of SEQ ID NO: 1. In another particular embodiment, the rat F-spondin protein of SEQ ID NO: 4 is encoded by a nucleic acid comprising the DNA sequence of SEQ ID NO: 2.

In another particular embodiment, the mouse F-spondin protein of SEQ ID NO: 6 is encoded by a nucleic acid comprising the DNA sequence of SEQ ID NO: 3. Moreover, the present invention provides for commercial test kits and assays for determining the presence of F-spondin in a biological sample.

For example, the assays and methods of the present invention broadly and generally include and incorporate the following steps in determining the presence of F-spondin in a subject: (a) isolation of nucleic acid from the subject; (b) amplification of at least a portion of the nucleic acid sequence; and (c) analysis of the sequence; or alternatively, isolating the F-spondin protein or a fragment thereof, from a tissue or cell sample from a subject and analyzing the level of F-spondin protein or a fragment thereof by standard procedures known to those skilled in the art.

In practicing the assays and methods of the present invention, it is necessary to perform a step to obtain, purify or otherwise isolate nucleic acid, DNA or mRNA for analysis. The term “isolation”, “isolating” or “isolate” as used herein, and as applied to the methods and assays described herein, refers to and encompasses any method or approach known in the art whereby DNA, RNA or a protein or peptide fragment can be obtained, procured, prepared, purified or isolated such that it is suitable for analysis, amplification, restriction enzyme cleavage and/or sequencing as provided in the methods and assays of the present invention. Various methods for the isolation or procurement of nucleic acid may be employed, as any skilled artisan may know and practice. Such methods may include methods employed for the isolation of genomic DNA, or mRNA in various forms and states of purity and may not necessarily involve or require the separation of DNA, RNA or protein from all cellular debris, protein, etc. The term isolation as used herein is contemplated to include the preparation of cell or tissue samples whereby DNA, RNA, or protein may be analyzed, amplified, etc. in situ. In the event mRNA is utilized, a first copy of DNA may be generated therefrom, for instance by reverse transcription using e.g. reverse transcriptase (RT), followed by amplification of the DNA copy or cDNA. Furthermore, an isolated nucleic acid molecule is one that is separated from other nucleic acids present in the natural source of the nucleic acid. The important point is that the nucleic acid is isolated from remote and unimportant flanking sequences and is of appropriate length such that it can be subjected to the specific manipulations or uses described herein such as recombinant expression, preparation of probes and primers, and other uses specific to the nucleic acid sequences. Moreover, an isolated nucleic acid molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated. For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The “amplification” or “amplifying” step may be performed utilizing any method of amplification, including polymerase chain reaction (PCR), ligase chain reaction (Barany, F. (1991) Proc. Natl. Acad. Sci. 88:189-193), rolling circle amplification (Lizardi, P. M. et al (1998) Nature Genetics 19:225-232), strand displacement amplification (Walker, G. T. et al (1992) Proc. Natl. Acad. Sci. 89:392-396) or alternatively any means or method whereby concentration or sequestration of sufficient amounts of the nucleic acid for analysis may be obtained. The primers for use in amplification of at least the 3′ untranslated region (UTR) of the F-spondin gene may be selected and utilized by the skilled artisan employing the sequence of any one of SEQ ID NOs: 1 (human), 3 (rat) or 5 (mouse) as available at the National Center for Biotechnology Information (NCBI) (www.ncbi.nlm.nih.gov) as GenBank numbers NM_{—}006108 (human); NM_{—}172067 (rat); and NM_{—}145884 (mouse). The proteins encoded by these nucleic acid sequences may be found in GenBank as accession numbers NP_{—}006099 (human F-spondin); NP_{—}742064.1 (rat F-spondin) and NP_{—}663559.1 (mouse F-spondin). These particular nucleic acid sequences may be utilized in the design and sequence of primers for diagnostic use as described herein.

Based on the nucleic acid sequences provided herein, PCR primers are constructed that are complementary to the nucleic acid encompassing the F-spondin gene. A primer consists of a consecutive sequence of polynucleotides complementary to any region in the nucleic acid encompassing the gene of interest, eg. F-spondin. The size of these amplification/PCR primers range anywhere from five bases to hundreds of bases. However, the preferred size of a primer is in the range from 10 to 50 bases, most preferably from 15 to 35, or 15 to 25 bases. As the size of the primer decreases, so does the specificity of the primer for the targeted region. Hence, even though a primer which is less than five bases long will bind to the targeted region, it also has an increased chance of binding to other regions of the template polynucleotide which are not in the targeted region and do not contain the polymorphic/mutated base. Conversely, a larger primer provides for greater specificity, however, it becomes quite cumbersome to make and manipulate a very large fragment. Nevertheless, when necessary, large fragments are employed in the method of the present invention. To amplify the region of the genomic DNA of the individual patient, primers to one or both sides of the targeted position are made and used in a PCR amplification reaction, using known methods in the art (e.g. Massachusetts General Hospital & Harvard Medical School, Current Protocols In Molecular Biology, Chapter 15 (Green Publishing Associates and Wiley-Interscience 1991) and as particularly exemplified herein.

The analysis or “measuring” or “detecting” or “determining” step will utilize skills and methods available to the skilled artisan for determining and distinguishing a sequence and can include: direct sequencing of the amplified or otherwise sequestered product; hybridization utilizing a labeled probe or labeled probe set; direct visualization of the PCR product by gel separation or by the presence of a non-radioactive dye or fluorescent dye introduced with the primer, particularly wherein allele specific oligonucleotide primers are utilized (including fluorescence as provided by the molecular beacon technology (Tyagi, S, and Kramer, F. (1996) Nature Biotech 14:303-308; Tyagi, S. et al (1998) Nature Biotech 16:49-53); restriction enzyme analysis wherein restriction enzyme cleavage is characteristic of the upstream regulatory region sequence; sequencing by hybridization, etc. As noted herein, the F-spondin protein can be isolated and quantified from a cell or tissue sample from a patient who is suspected of having or prone to developing, a cartilage degenerative condition. The specific methods for measuring, or detecting, or determining, the level of the F-spondin gene or gene product (protein) are described herein.

### Nucleic Acid and Protein Sequences Useful in the Invention

The invention provides for the identification of elevated levels of expression of the F-spondin gene or gene product in patients suffering from osteoarthritis or other cartilage degenerative conditions as compared to the level observed in normal patients In one particular embodiment, the level of f-spondin in patients suffering from osteoarthritis or another cartilage degenerative condition is increased by 1.5 times, or 5 times, or 10 times or 90 times the level observed in normal patients (eg. patients not suffering from osteoarthritis or a cartilage degenerative condition), when measured by quantitative PCR. In another particular embodiment, the particular nucleic acid that is elevated in OA patients compared to normals is set forth in SEQ ID NO: 1 (GenBank accession number NM_{—}006108). In yet another particular embodiment, the particular protein that is elevated in OA patients compared to normals is set forth in the amino acid sequence of SEQ ID NO: 2 (GenBank accession number NP_{—}006099). The corresponding sequences found in rat and mouse are described above and are found in SEQ ID NOs: 3 and 4 for rat and SEQ ID NOs: 5 and 6 for mouse. The invention also provides methods of detecting or measuring a target nucleic acid sequence, such as the nucleic acid comprising F-spondin described herein; more particularly SEQ ID NO: 1, and also utilizes specific oligonucleotide primers for amplifying a particular template nucleic acid sequence and specific probes for identifying the target sequence. The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength, the temperature, and incidence of mismatched base pairs. The oligonucleotide is not necessarily physically derived from any existing or natural sequence but may be generated in any manner, including chemical synthesis, DNA replication, reverse transcription or a combination thereof.

When two different, non-overlapping oligonucleotides anneal to different regions of the same linear complementary nucleic acid sequence, and the 3′ end of one oligonucleotide points toward the 5′ end of the other, the former may be called the “upstream” annealed oligonucleotide and the latter the“downstream” annealed oligonucleotide.

### Nucleic Acid Probes and Primers Useful for Practicing the Methods of the Invention

The invention provides specific nucleic acid sequences from which oligonucleotide primers and probes may be prepared for detecting or measuring F-spondin, in particular, the nucleic acid sequences of SEQ ID NOs: 1, 3 and 5. In one particular embodiment, the probe utilized for the studies presented herein was obtained from Applied Biosystem (spon1 human probe Hs00391824_ml). Oligonucleotide primers useful according to the invention may be single-stranded DNA or RNA molecules that are hybridizable to a template nucleic acid sequence and prime enzymatic synthesis of a second nucleic acid strand. The primer is complementary to a portion of a target molecule present in a pool of nucleic acid molecules. It is contemplated that oligonucleotide primers according to the invention may be prepared by synthetic methods, either chemical or enzymatic. Alternatively, such a molecule or a fragment thereof may be naturally-occurring, and is isolated from its natural source or purchased from a commercial supplier. Oligonucleotide primers and probes are generally 5 to 100 nucleotides in length, ideally from 10 to 50 nucleotides, although primers and probes of different lengths may also be used. Primers for amplification are preferably about 15-25 nucleotides. Primers useful according to the invention are also designed to have a particular melting temperature (Tm) by the method of melting temperature estimation. Commercial programs, including Oligo™, Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65′ C and more preferably between about 50° and 60° C. Preferably, the Tm of a probe useful according to the invention is 7° C. higher than the Tm of the corresponding amplification primers.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of the primer to a second nucleic acid molecule. These factors, which include primer length, nucleotide sequence and/or composition, hybridization temperature, buffer composition and potential for steric hindrance in the region to which the primer is required to hybridize, will be considered when designing oligonucleotide primers according to the invention.

A positive correlation exists between primer length and both the efficiency and accuracy with which a primer will anneal to a target sequence. In particular, longer sequences have a higher melting temperature (T_{M}) than do shorter ones, and are less likely to be repeated within a given target sequence, thereby minimizing promiscuous hybridization. Primer sequences with a high G-C content or that comprising palindromic sequences tend to self-hybridize, as do their intended target sites, since unimolecular, rather than bimolecular, hybridization kinetics are generally favored in solution. However, it is also important to design a primer that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C., greater than about 3° C., and (most often) in excess of about 37° C. Longer fragments may require higher hybridization temperatures for specific hybridization. As several factors affect the stringency of hybridization, the combination of parameters is more important than the absolute measure of a single factor.

Oligonucleotide primers can be designed with these considerations in mind and synthesized according to the following methods.

### Oligonucleotide Primer Design Strategy

The design of a particular oligonucleotide primer for the purpose of sequencing, PCR, or for use in identifying target nucleic acid molecules involves selecting a sequence that is capable of recognizing the target sequence, but has a minimal predicted secondary structure. The oligonucleotide sequence binds only to a single site in the target nucleic acid sequence. Furthermore, the Tm of the oligonucleotide is optimized by analysis of the length and GC content of the oligonucleotide.

The design of a primer is facilitated by the use of readily available computer programs, developed to assist in the evaluation of the several parameters described above and the optimization of primer sequences. Examples of such programs are “Primer Express” (Applied Biosystems), “PrimerSelect” of the DNAStar™. “PrimerSelect” of the DNAStar™ software package (DNAStar, Inc.; Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3rd Edition, John Wiley & Sons).

It is well known by those with skill in the art that oligonucleotides can be synthesized with certain chemical and/or capture moieties, such that they can be coupled to solid supports. Suitable capture moieties include, but are not limited to, biotin, a hapten, a protein, a nucleotide sequence, or a chemically reactive moiety. Such oligonucleotides may either be used first in solution, and then captured onto a solid support, or first attached to a solid support and then used in a detection reaction. An example of the latter would be to couple a downstream probe molecule to a solid support, such that the 5′ end of the downstream probe molecule comprised a fluorescent quencher. The target nucleic acid could hybridize with the solid-phase downstream probe oligonucleotide, and a liquid phase upstream primer could also hybridize with the target molecule. This would cause the solid support-bound fluorophore to be detectable. Different downstream probe molecules could be bound to different locations on an array. The location on the array would identify the probe molecule, and indicate the presence of the template to which the probe molecule can hybridize.

The primers themselves are synthesized using techniques that are also well known in the art. Methods for preparing oligonucleotides of specific sequence are known in the art, and include, for example, cloning and restriction digest analysis of appropriate sequences and direct chemical synthesis. Once designed, oligonucleotides are prepared by a suitable chemical synthesis method, including, for example, the phosphotriester method described by Narang et al., 1979, Methods in Enzymology, 68:90, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using either a commercial automated oligonucleotide synthesizer (which is commercially available) or VLSIPS™ technology.

### Probes

The invention provides for probes useful for identifying sequences specific for the F-spondin gene.

As used herein, the term “probe” refers to a labeled oligonucleotide primer which forms a duplex structure with a sequence in the target nucleic acid, due to complementarity of at least one sequence in the probe with a sequence in the target region. The probe, preferably, does not contain a sequence complementary to sequence(s) used in the primer extension (s). Generally the 3′ terminus of the probe will be “blocked” to prohibit incorporation of the probe into a primer extension product. “Blocking” can be achieved by using non-complementary bases or by adding a chemical moiety such as biotin or a phosphate group to the 3′ hydroxl of the last nucleotide, which may, depending upon the selected moiety, serve a dual purpose by also acting as a label for subsequent detection or capture of the nucleic acid attached to the label. Blocking can also be achieved by removing the 3′-OH or by using a nucleotide that lacks a 3′-OH such as dideoxynucleotide.

In certain embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will be of particular utility. Longer contiguous identical or complementary sequences, e.g., those of about 20, 30, 40, 50, 100, 200, 500, 1000 (including all intermediate lengths) and even up to full length sequences also be of use in certain embodiments.

The ability of such nucleic acid probes to specifically hybridize to a sequence of interest will enable them to be of use in detecting the presence of complementary sequences in a given sample.

Polynucleotide molecules having sequence regions consisting of contiguous nucleotide stretches of 10-14, 15-25, 30, 50, or even of 100-200 nucleotides or so (including intermediate lengths as well), identical or complementary to a polynucleotide sequence disclosed herein, are particularly contemplated as hybridization probes for use in PCR assays. This would allow a gene product, or fragment thereof, to be analyzed, in various samples, including but not limited to biological samples. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequence set forth in SEQ ID NOs: 1, 3 or 5 or to any continuous portion of the sequence, from about 15-25 nucleotides in length up to and including the full length sequence, that one wishes to utilize as a probe or primer.

Small polynucleotide segments or fragments may be readily prepared by, for example, directly synthesizing the fragment by chemical means, as is commonly practiced using an automated oligonucleotide synthesizer.

For hybridization techniques, a partial sequence may be labeled (e.g., by nick-translation or end-labeling with ³²P) using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in a known region of a gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Probes of the present invention may also have one or more detectable markers attached to one or both ends. The marker may be virtually any molecule or reagent which is capable of being detected, representative examples of which include radioisotopes or radiolabeled molecules, fluorescent molecules, fluorescent antibodies, enzymes, or chemiluminescent catalysts. Within certain embodiments of the invention, the probe may contain one or more labels such as a fluorescent or enzymatic label (e.g., quenched fluorescent pairs, or, a fluorescent label and an enzyme label), or a label and a binding molecule such as biotin (e.g., the probe, either in its cleaved or uncleaved state, may be covalently or non-covalently bound to both a label and a binding molecule (see also, e.g., U.S. Pat. No. 5,731,146).

As noted above, the probes of the present invention may also be linked to a solid support either directly, or through a chemical linker. Representative examples of solid supports include silicaceous, cellulosic, polymer-based, or plastic materials.

Methods for constructing such nucleic acid probes may be readily accomplished by one of ordinary skill in the art, given the disclosure provided herein. Particularly preferred methods are described for example by: Matteucci and Caruthers, J. Am. Chem. Soc. 103:3185, 1981; Beaucage and Caruthers, Tetrahedron Lett. 22:1859-1862, 1981; U.S. Pat. Nos. 4,876,187 and 5,011,769; Ogilvie et al., Proc. Natl. Acad. Sci. USA 85:8783-8798, 1987; Usman et al., J. Am. Chem. Soc. 109:7845-7854, 1987; Wu et al., Tetrahedron Lett. 29:4249-4252, 1988; Chaix et al., Nuc. Acids Res. 17:7381-7393, 1989; Wu et al., Nuc. Acids Res. 17:3501-3517, 1989; McBride and Caruthers, Tetrahedron Lett. 24:245-248, 1983; Sinha et al., Tetrahedron Lett. 24:5843-5846, 1983; Sinha et al., Nuc. Acids Res. 12:4539-4557, 1984; and Gasparutto et al., Nuc. Acids Res. 20:5159-5166, 1992.

The probes of the preferred embodiment are based on and or derived from the human F-spondin gene of SEQ ID NO: 1. Moreover, the rat F-spondin nucleic acid is set forth in SEQ ID NO: 3 and the mouse F-spondin nucleic acid sequence is found in SEQ ID NO: 5.

### Detection Reactions

A wide variety of cycling reactions for the detection of a desired target nucleic acid molecule, such as the human F-spondin gene associated with the onset of osteoarthritis, or other cartilage degenerative conditions may be readily performed according to the general procedures set forth above (see also, U.S. Pat. Nos. 5,011,769 and 5,403,711).

In another embodiment, Cycle ProbeTechnology (CPT) can be used for detecting amplicons generated by any target amplification technology. For example CPT enzyme immunoassay (CPT-EIA) can be used for the detection of PCR amplicons. CPT allows rapid and accurate detection of PCR amplicons. CPT adds a second level of specificity which will prevent detection of non-specific amplicons and primer-dimers. The PCR-CPT method may also be used for mismatch gene detection.

Other variations of this assay include ‘exponential’ cycling reactions such as described in U.S. Pat. No. 5,403,711 (see also U.S. Pat. No. 5,747,255).

A lateral flow device (strip or dipstick) as described in U.S. Pat. Nos. 4,855,240 and 4,703,017, for example, represents another embodiment used in the assay for detecting the F-spondin gene. Instead of detecting an uncleaved F-spondin probe on streptavidin coated wells (i.e., EIA format), the uncleaved probe is captured by streptavidin impregnated on a membrane (i.e., strip format). There are several advantages for using this format. There are no additional detection reagents required, less hands-on time, and a short detection time. Representative examples of further suitable assay formats including any of the above assays which are carried out on solid supports such as dipsticks, magnetic beads, and the like (see generally U.S. Pat. Nos. 5,639,428; 5,635,362; 5,578,270; 5,547,861; 5,514,785; 5,457,027; 5,399,500; 5,369,036; 5,260,025; 5,208,143; 5,204,061; 5,188,937; 5,166,054; 5,139,934; 5,135,847; 5,093,231; 5,073,340; 4,962,024; 4,920,046; 4,904,583; 4,874,710; 4,865,997; 4,861,728; 4,855,240; 4,847,194 and 6,130,098).

In another embodiment, CPT can be carried out using the exponential formats with two sets of nucleic acid probe molecules, which are immobilized on solid support as described in U.S. Pat. No. 5,403,711. This would be advantageous since the assay can be carried out in a single container, the signal can be monitored over time and would result in a very rapid and sensitive assay.

In yet another embodiment, CPT-EIA can be used for detecting the F-spondin gene by use of reverse transcriptase to transcribe cDNA from mRNA expressed by the F-spondin gene followed by Cycling Probe Technology (RT-CPT) as described in U.S. Pat. No. 5,403,711. The uncleaved probe specific for the cDNA can than be detected by EIA.

In the area of DNA diagnostics, automated platforms based on labeled synthetic oligonucleotides immobilized on silicon chips work by fluorescence detection and are capable of the parallel analysis of many samples and mutations. Methods for preparing labeled, chemically activated nucleotide precursors for oligonucleotide synthesis are known to those skilled in the art. Nucleic acid amplification methods such as PCR have become very important in genetic analysis and the detection of trace amounts of nucleic acid from pathogenic bacteria and viruses. Analysis of many PCR reactions by standard electrophoretic methods becomes tedious, time consuming and does not readily allow for rapid and automated data acquisition. PCR has been adapted for use with fluorescent molecules by incorporation of fluorescently labeled primers or nucleotides into the PCR product which is then directly detected or detected indirectly using secondary probes, the binding of which is detectable. Removal of unincorporated, labeled substrates is usually necessary and can be accomplished by filtration, electrophoretic gel purification or chromatographic methods. However, the large amount of sample handling required by these analytical techniques make these purification methods labor intensive, not quantitative and they invariably leads to serious contamination problems. Affinity capture of PCR products by strepavidin coated beads or micro titer wells requires incorporation of biotin labels in addition to the fluorophores and still involves transfer steps that can lead to contamination. Instrumentation utilizing both gel electrophoresis and laser excitation optics represents an improvement in data acquisition but cannot handle large numbers of samples, retains the comparatively prolonged separation times characteristic of gels and still requires sample transfer.

### Polynucleotide Amplification Techniques

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™ bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Q beta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site, may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of a second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, discloses a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

The invention also provides a kit for generating a signal indicative of the presence of a target nucleic acid sequence in a sample, such as the human F-spondin gene as described herein, comprising a nucleic acid polymerase, a primer, a probe and a suitable buffer. In a preferred embodiment, the invention also provides a kit for generating a signal indicative of the presence of a target nucleic acid sequence from human F-spondin in a sample comprising one or more nucleic acid polymerases, primers and probes and a suitable buffer. In a preferred embodiment, the target nucleic acid sequence is the human F-spondin nucleic acid of SEQ ID NO: 1 or a fragment thereof.

In another preferred embodiment the kit further comprises a labeled nucleic acid complementary to the target nucleic acid sequence.

Further features and advantages of the invention are as follows. The claimed invention provides a method of generating a signal to detect and/or measure a target nucleic acid wherein the generation of a signal is an indication of the presence of human F-spondin as a target nucleic acid in a sample. The claimed invention also provides a PCR based method for detecting and/or measuring a target nucleic acid comprising generating a signal as an indication of the presence of a target nucleic acid. The claimed invention allows for amplification and detection and/or measurement of a target nucleic acid sequence, such as that set forth in SEQ ID NO: 1.

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

### Polymerase Chain Reaction (PCR)

Nucleic acids of the invention may be amplified from genomic DNA or other natural sources by any of the known methods of polymerase chain reaction (PCR). PCR methods are well-known to those skilled in the art.

PCR, may be performed as described in Mullis and Faloona, 1987, Methods Enzymol., 155: 335, herein incorporated by reference.

The polymerase chain reaction (PCR) technique, is also disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 10⁹. The PCR method is also described in Saiki et al., 1985, Science 230:1350.

In a particular embodiment of the present invention, the PCR procedure may be a real-time PCR procedure. Moreover, the PCR procedure employed may use the materials and methodology outlined in U.S. Pat. No. 6,130,098, incorporated herein by reference in its entirety.

Detection methods generally employed in standard PCR techniques use a labeled probe with the amplified DNA in a hybridization assay. Preferably, the probe is labeled, e.g., with ³²P, biotin, horseradish peroxidase (HRP), etc., to allow for detection of hybridization.

In a particular embodiment of the present invention, the probe utilized recognizes the sequence amplified for the human F-spondin gene comprising the sequence of SEQ ID NO: 1, allowing real-time detection by using fluorescence measurements

Other means of detection include the use of fragment length polymorphism (PCR FLP), hybridization to allele-specific oligonucleotide (ASO) probes (Saiki et al., 1986, Nature 324:163), or direct sequencing via the dideoxy method (using amplified DNA rather than cloned DNA). The standard PCR technique operates (essentially) by replicating a DNA sequence positioned between two primers, providing as the major product of the reaction a DNA sequence of discrete length terminating with the primer at the 5′ end of each strand. Thus, insertions and deletions between the primers result in product sequences of different lengths, which can be detected by sizing the product in PCR-FLP. In an example of ASO hybridization, the amplified DNA is fixed to a nylon filter (by, for example, UV irradiation) in a series of “dot blots”, then allowed to hybridize with an oligonucleotide probe labeled with HRP under stringent conditions. After washing, terramethylbenzidine (TMB) and hydrogen peroxide are added: HRP oxidizes the hydrogen peroxide, which in turn oxidizes the TMB to a blue precipitate, indicating a hybridized probe.

### Oligonucleotide Design for Real-Time PCR Assays

There are several different approaches to real-time PCR. SYBR green detection is utilized with real time PCR because multiple reactions can be set-up rapidly and inexpensively using standard oligonucleotides. Real-time PCR relies on the fluorescent quantification of PCR product during each cycle of amplification. Specific detection systems, such as molecular beacons and Taqman assays rely on the synthesis of a fluorescently labeled detection oligonucleotide. These specific assays have the advantage of specificity. Assay of PCR product through the use of the fluorescent dye SYBR green allows the reaction to be based on standard oligonucleotides. Because SYBR green will detect any PCR product, including non-specific products and primer-dimers, careful oligonucleotide design for the reaction is required.

Primers should be designed, if possible, within 1 kb of the polyadenylation site. Amplicons of 100-200 by are ideal for real time applications. It is advantageous to design the primers to have the same melting temperature so that PCR with different primer sets can be performed in the same run. Primers that are 20-mers with 55% GC content and a single 3′-G or C can be used. Candidate primers are tested for specificity by BLAST and for folding and self annealing using standard DNA analysis software. Primer pairs are first tested for specificity and absence of primer-dimer formation (low molecular weight products) by PCR followed by gel electrophoresis. Designing each primer pair takes about one hour.

### Real Time PCR

Real-time PCR requires a specialized thermocycler with fluorescent detection. A variety of commercial instruments are available. The ABI Prism 7700 allows assays to be performed in 96 well plate format. Good PCR technique is required to avoid contamination of subsequent reactions. This includes isolating PCR products and plasmids from RNA preparation and reaction setup. A dedicated bench for RNA isolation and PCR reaction set-up and dedicated pipettors should be maintained. Aerosol resistant pipette tips are used.

Commercial kits for SYBR green based PCR reactions are available from Applied Biosystems and perform reliably (SYBR Green PCR Core Reagents, P/N 4304886; SYBR Green PCR Master Mix, P/N 4309155).

“Hot start” taq polymeraase may be used. Platinum Taq, (Life Technologies), and Amplitaq gold, (Applied Biosystems), both perform well. The 10×SYBR Green I may be prepared by diluting 10 μl of the stock 10,000× concentrate (Cat#S-7563, Molecular Probes, Eugene, Oreg.) into 10 ml Tris-HCl, pH 8.0, and is stored in 0.5 ml aliquots at −20° C.

Accordingly, the present invention resides in part in a method for identifying the human F-spondin gene by isolating a nucleic acid sample from a subject, amplifying the nucleic acid present in the sample, and assaying for the presence of the nucleic acid of SEQ ID NO: 1 in the sample. The presence of the gene indicates a likelihood that the patient is suffering from osteoarthritis, or a related cartilage degenerative condition, or serves as a biomarker or predictor of the risk for development of osteoarthritis in the subject, or a related cartilage degenerative condition.

### Detection Kits, Nucleic Acid Arrays and Integrated Systems

The present invention further provides F-spondin detection reagents and kits, such as arrays/microarrays of nucleic acid molecules, or probe/primer sets, and other detection reagent sets, that are based on the sequences provided in the Sequence Listing. In a more specific embodiment, the kits will contain the PCR oligonucleotide primers capable of selectively hybridizing to a nucleic acid encoding a human F-spondin gene.

In one embodiment of the present invention, kits are provided which contain the necessary reagents to carry out one or more assays that detect the F-spondin gene.

Specifically, the invention provides a compartmentalized kit to receive, in close confinement, one or more containers which comprises: (a) a first container comprising one or more nucleic acid probes, that can bind to a fragment of the human genome containing the F-spondin gene; and (b) one or more other containers comprising one or more of the following: wash reagents or reagents capable of detecting the presence of a bound probe. Containers may be interchangeably referred to as, for example, “compartments”, “chambers”, or “channels”.

In detail, a compartmentalized kit includes any kit in which reagents are contained in separate containers. Such containers include small glass containers, plastic containers, strips of plastic, glass or paper, or arraying material such as silica. Such containers allow one to efficiently transfer reagents from one compartment to another compartment such that the samples and reagents are not cross-contaminated, and the agents or solutions of each container can be added in a quantitative fashion from one compartment to another. Such containers may include a container which will accept the test sample, a container which contains the probe, containers which contain “other reagents” such as wash reagents (such as phosphate buffered saline, Tris-buffers, etc.), and containers which contain “other reagents” used to detect the bound probe. The kit can further comprise “other reagents” known to those skilled in the art for PCR or other enzymatic reactions, and instructions for using the kit.

As used herein “Arrays” or “Microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522. Arrays or microarrays are commonly referred to as “DNA chips”. As used herein, arrays/microarrays may be interchangeably referred to as detection reagents or kits.

Any number of oligonucleotide probes may be implemented in an array. The oligonucleotides are synthesized at designated areas on a substrate using a light-directed chemical process. The substrate may be paper, nylon or other type of membrane, filter, chip, glass slide or any other suitable solid support.

Hybridization assays based on oligonucleotide arrays rely on the differences in hybridization stability of short oligonucleotides probes to perfectly matched and mismatched target sequence variants. Preferably, probes are attached to a solid support in an ordered, addressable array.

The test samples of the present invention include, but are not limited to, nucleic acid extracts, cells, and protein or membrane extracts from cells, which may be obtained from any bodily fluids (such as blood, urine, saliva, synovial fluid, serum, plasma, etc.), cultured cells, biopsies, or other tissue preparations, such as cartilage. The test sample used in the above-described methods will vary based on the assay format, nature of the detection method and the tissues, cells or extracts used as the sample to be assayed. Methods of preparing nucleic acid, protein, or cell extracts are well known in the art and can be readily be adapted in order to obtain a sample that is compatible with the system utilized.

### Diagnostic Tools, Assays and Methods for Identifying Novel Therapeutics

One aspect of the invention provides a means of determining whether a subject is responsive to treatment with an agent useful for treating osteoarthritis or other cartilage degenerative conditions, or of assessing the final outcome of therapy with such agent. Another aspect of the invention provides for the use of methods for screening for novel therapeutics for treating a disease associated with F-spondin expression, such as OA and related cartilage degenerative conditions. A further aspect provides for the use of methods for screening for novel therapeutics for treating cartilage and/or bone diseases disorders or conditions, including chondrodyplasia dwarfism, bone fractures, osteoporosis, bone cancer. A still further aspect of the invention provides for the use of methods for screening for novel therapeutics for treating a disease or in situations or under conditions wherein cartilage is replaced by bone or bone growth is warranted or necessary. Modulation of F-spondin may be used in the modulation, alleviation or treatment of diseases of the transient growth cartilage of the long bones, including chondrodysplasias and dwarfism, on in bone diseases, bone degeneration, bone fractures or bone cancer where replacement of bone or endochondral bone formation is warranted or helpful. The methods described herein are merely exemplary and are not meant to be limiting, and as such, it is to be recognized that one of skill in the art would be cognizant of the various other methodologies that may be used to determine effectiveness of therapy with such agents, or to identify novel analogues or derivatives or metabolites of such agents for use in treating OA or other cartilage degenerative conditions.

In another particular embodiment, such a method comprises determining the levels of expression of one or more genes or gene products (proteins) which are modulated in a cell of the subject undergoing treatment with an agent useful for treating OA or a related cartilage degenerative condition or a bone degenerative or bone disorder or injury condition and comparing these levels of expression with the levels of expression of the genes and gene products in a cell of a subject not treated with such agent, or of the same subject before treatment with such agent, such that the modulation (either up or down-regulation of the gene or gene product) of one or more genes is indicative that the subject is responsive to treatment with such agent. In one embodiment, the cell is obtained from a sample of whole blood, for example, white blood cells, including lymphocytes, monocytes, neutrophils and the like, although other cells expressing these genes are also contemplated for analysis. Other samples useful for analysis include urine, bone marrow, cerebrospinal fluid, saliva, chondrocytes, cartilage, synovium and synovial fluid. Samples from sites of cartilage damage or bone injury or disease, including cancer may be utilized. In one particular embodiment, the gene or gene product is the F-spondin nucleic acid, or protein, or a fragment thereof. In another embodiment, the gene may be any one or more of the genes or gene products from the PGE2, TGF-β or αvβ3 pathways. In yet another embodiment, the gene or gene product may be any one or more selected from the group consisting of COL2A, aggrecan, MMP-13, BMP2, PGE2, MMP-1, IL-8, Il-1β and TNF-α; or with activation of latent TGF-β1. A person of skill in the art will recognize that in certain diagnostic and prognostic assays, it will be sufficient to assess the level of expression of a single gene as noted above and that in others, the expression of two or more is preferred. For example, the level of expression of a gene or gene product (protein) may be determined by a method selected from, but not limited to, cDNA microarray, reverse transcription-polymerase chain reaction (RT-PCR), real time PCR and proteomics analysis. Other means such as electrophoretic gel analysis, enzyme immunoassays (ELISA assays), immunohistochemistry, Western blots, dotblot analysis, Northern blot analysis and in situ hybridization may also be contemplated for use, although it is to be understood that the former assays that are noted (eg. microarrays, RT-PCR, real time PCR and proteomics analysis) provide a more sensitive, quantitative and reliable measurement of genes or gene products that are modulated by an agent useful for treating OA or a related cartilage degenerative condition. Sequences of the genes or cDNA from which probes are made (if needed) for analysis may be obtained, e.g., from GenBank, other public databases or publications. Magnetic resonance imaging may also be used for assessing the effect of an agent useful for treating OA or a related condition on expression of F-spondin or other protein biomarkers.

In another particular embodiment, novel candidate therapeutics may be tested for activity by measuring their effect on expression of F-spondin, as described herein. The candidate therapeutics may be selected from the following classes of compounds: proteins, peptides, peptidomimetics, antibodies, derivatives of fatty acids, nucleic acids, including DNA or RNA, antisense molecules or siRNA molecules, or other small organic molecules, either synthetic or naturally derived. In some embodiments, the candidate therapeutics are selected from a library of compounds. These libraries may be generated using combinatorial synthetic methods.

### Use of Microarrays for Determining Gene Expression Levels

Microarrays may also be used for determining gene expression levels and may be prepared by methods known in the art, or they may be custom made by companies, e.g., Affymetrix (Santa Clara, Calif.) (see www.affymetrix.com). Numerous articles describe the different microarray technologies, (e.g., Shena, et al., Tibtech, (1998), 16: 301; Duggan, et al., Nat. Genet., (1999), 21:10; Bowtell, et al., Nat. Genet., (1999), 21:25; Hughes, et al., Nat. Biotechn., (2001), 19:342). While many of the microarrays utilize nucleic acids and relevant probes for the analysis of gene expression profiles, protein arrays, in particular, antibody arrays or glycosylation arrays also hold promise for studies related to protein or glycoprotein expression from biological samples (see for example, RayBiotech, Inc. at www.raybiotech.com/product.htm, Panomics at www.panomics.com, Clontech Laboratories, inc. at www.clontech.com, Procognia in Maidenhead, UK and Qiagen at www.qiagen.com.

### Samples for Analysis

While the efficacy of an agent useful for treating OA or a related cartilage degenerative condition may be tested in a subject for its effect on, for example, decreased pain, or decreased swelling, or an increase in mobility, it may also be of interest to assess its effects on the modulation of the F-spondin gene or gene product, or the other genes or gene products noted above. While it may be possible to look at the level of a particular gene in certain cellular samples (whole blood cells or peripheral blood mononuclear cells), a more particular method would involve the analysis of the protein expression in these cell types or in the plasma, serum, urine, or synovial fluid from the subjects exposed or treated with such agent. For example, protein and nucleic acids prepared from specimens may be obtained from an individual to be tested using either “invasive” or “non-invasive” sampling means. A sampling means is said to be “invasive” if it involves the collection of the biosamples from within the skin or organs of an animal (including, especially, a human, a murine, an ovine, an equine, a bovine, a porcine, a canine, or a feline animal). Examples of invasive methods include needle biopsy, pleural aspiration, etc. Examples of such methods are discussed by Kim, C. H. et al., J. Virol., (1992), 66:3879-3882; Biswas, B. et al., Annals NY Acad. Sci., (1990), 590:582-583; Biswas, B., et al., J. Clin. Microbiol., (1991), 29:2228-2233.

In one embodiment the assays of the present invention will be performed on cells including but not limited to whole blood cells, or isolated white blood cells from a mammal, or from cell cultures propagated for laboratory purposes, eg. chondrocytes. Primary cultures or cell lines can be used. Appropriate cell lines that can be obtained for screening purposes are commercially available from the ATCC. In yet another embodiment, a sample of whole blood, blood plasma or serum is obtained for further analysis.

### Other Methods for Determining Gene Expression Levels

In certain embodiments, it is sufficient to determine the expression of one or only a few genes, as opposed to hundreds or thousands of genes. Although microarrays may be used in these embodiments, various other methods of detection of gene expression are available.

For example, the modulation of gene expression can be performed using a RT-PCR or Real Time-PCR assay. Total RNA is extracted using procedures known to those skilled in the art and subjected to reverse transcription using an RNA-directed DNA polymerase, such as reverse transcriptase isolated from AMV, MoMuLV or recombinantly produced. The cDNAs produced can be amplified in the presence of Taq polymerase and the amplification monitored in an appropriate apparatus in real time as a function of PCR cycle number under the appropriate conditions that yield measurable signals, for example, in the presence of dyes that yield a particular absorbance reading when bound to duplex DNA. The relative concentrations of the mRNAs corresponding to chosen genes can be calculated from the cycle midpoints of their respective Real Time-PCR amplification curves and compared between cells exposed to a candidate therapeutic relative to a control cell in order to determine the increase or decrease in mRNA levels in a quantitative fashion.

In addition, a method for high throughput analysis of gene expression is the serial analysis of gene expression (SAGE) technique, first described in Velculescu, et al., Science, (1995), 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that may be used to identify the detected genes. Thus far, SAGE methodology has proved itself to reliably detect expression of regulated and nonregulated genes in a variety of cell types (Velculescu, et al., (1997), Cell, 88, 243-251; Zhang, et al., Science, (1997), 276, 1268-1272 and Velculescu, et al., Nat Genet, (1999), 23, 387-388. Techniques for producing and probing nucleic acids are further described, for example, in Sambrook, et al., Molecular Cloning: A Laboratory Manual (New York, Cold Spring Harbor Laboratory, 1989).

In other methods, the level of expression of a gene is detected by measuring the level of protein encoded by the gene. In the case of polypeptides which are secreted from cells, the level of expression of these polypeptides may be measured in biological fluids. While methods such as immunoprecipitation, ELISA, Western blot analysis, or immunohistochemistry using an agent, e.g., an antibody, that specifically detects the protein encoded by the gene may be contemplated, other more sensitive and quantitative methods are preferred, as described below. The invention is not limited to a particular assay procedure, and therefore is intended to include both homogeneous and heterogeneous procedures. General techniques to be used in performing the various immunoassays noted above are known to those of ordinary skill in the art. Antibodies useful for measuring F-spondin or fragments may be prepared using standard procedures known to those skilled in the art, or may be purchased from, for example, GenWay (15-288-22651), which is a chicken anti-spondin antibody; from Novus Biologicals (H00010418-M01), which is a mouse anti-human F-spondin clone 3F4; and GeneTex (GTX14271), which is a chicken anti-spondin 1 antibody.

### Proteomics: Rationale for Use

While genomic profiling provides information about susceptibility to disease, proteomic profiling reflects snapshots of metabolic dynamics, reveals heterogeneous gene expression, identifies biologically relevant phenotypes and generates information on protein structure-function relationships in the severity and prognosis of a disease. Thus, results from proteomic studies should offer insight into the pathology of OA and effects by therapy that modifies expression of F-spondin. Many forms of protein alterations can be associated with pathophysiological changes and therapeutic treatments. In addition to expression levels and patterns, these include alternative splicing, post-translational modifications, proteolytic processes, co-secretion and protein-protein interactions. Thus, the identification and quantification of proteins alone is not sufficient to understand functional interactions. Changes as small as the addition of a single phosphate, cleavage of a leader peptide, amidation, or oxidation, can drastically alter the biological function of a protein. Thus, it is important to detect these minute changes using sensitive and accurate proteomic technology.

### Proteomics: The Use of 2DE, MS, MALDI-TOF, MS/MS, LC/MS, and SELDI-TOF

2-dimensional polyacrylamide gel electrophoresis (2DE) coupled to mass spectrometry (MS) is currently the standard analysis in proteomics. Plasma samples may be subjected to 2DE (first dimension isoelectic focusing, second dimension SDS-PAGE). Selected spots from 2DE may be extracted from the gels, digested with trypsin and subjected to MS analysis to determine their identities (Aebersold, R. & Mann, M. Mass spectrometry-based proteomics. Nature 422, 198-207 (2003)). MALDI-TOF (matrix-assisted laser desorption/ionization coupled with time of flight (TOF)) is a method of choice to be used for proteins (Tanaka, K. The origin of macromolecule ionization by laser irradiation (Nobel lecture). Angew Chem Int Ed Eng142, 3860-70 (2003).). Tandem MS (MS/MS) may be used for selective isolation of peptide fragments to read out the (partial) amino acid sequence, and LC/MS (liquid chromatography coupled to MS) may be used for the identification of small peptides. However, the 2DE/MS detection is restricted to pI between 4 and 10 and proteins within an MW range of 10-200 kDa. Thus, peptides or small proteins (0.5-10 kDa), which may be related to OA pathogenesis may not be detected by 2DE/MS. Thus, additional initial separation systems such as C/MS using HPLC coupled MALDI-TOF for differential small peptide display is contemplated for use (America, A. H., Cordewener, J. H., van Geffen, M. H., Lommen, A., Vissers, J. P., Bino, R. J. & Hall, R. D. Alignment and statistical difference analysis of complex peptide data sets generated by multidimensional LC-MS. Proteomics 6, 641-53 (2006). Surface enhanced laser desorption ionization and time of flight (SELDI-TOF) using chromatographic chip surfaces based on amino acid sequence, protein structure, charge or hydrophobicity is also contemplated for use (Weinberger, S. R., Dalmasso, E. A. & Fung, E. T. Current achievements using ProteinChip Array technology. Curr Opin Chem Biol 6, 86-91 (2002)), as well as antibody proteomics based on immunoaffinity (Ingvarsson, J., Lindstedt, M., Borrebaeck, C. A. & Wingren, C. One-step fractionation of complex proteomes enables detection of low abundant analytes using antibody-based microarrays. J Proteome Res 5, 170-6 (2006)).

### Kits

In specific embodiments, in a screening assay described herein, the amount of protein or RNA product of a biomarker of the invention is determined utilizing kits. Such kits comprise materials and reagents required for measuring the expression of at least one or more proteins or nucleic acids encoding these proteins, or all or any combination of the biomarkers of the invention. In specific embodiments, the kits may further comprise one or more additional reagents employed in the various methods, such as: (1) reagents for purifying RNA from blood, chondrocytes or synovial fluid; (2) primers for generating test nucleic acids; (3) dNTPs and/or rNTPs (either premixed or separate), optionally with one or more uniquely labeled dNTPs and/or rNTPs (e.g., biotinylated or Cy3 or Cy5 tagged dNTPs); (4) post synthesis labeling reagents, such as chemically active derivatives of fluorescent dyes; (5) enzymes, such as reverse transcriptases, DNA polymerases, and the like; (6) various buffer mediums, e.g., hybridization and washing buffers; (7) labeled probe purification reagents and components, like spin columns, etc.; and (8) protein purification reagents; (9) signal generation and detection reagents, e.g., streptavidin-alkaline phosphatase conjugate, chemifluorescent or chemiluminescent substrate, and the like. In particular embodiments, the kits comprise prelabeled quality controlled protein and or RNA transcript (preferably, mRNA) for use as a control.

In some embodiments, the kits are RT-PCR kits. In other embodiments, the kits are nucleic acid arrays and protein arrays. Such kits according to the subject invention will at least comprise an array having associated protein or nucleic acid members of the invention and packaging means therefore. Alternatively the protein or nucleic acid members of the invention may be prepackaged onto an array.

In a specific embodiment, kits for measuring a RNA product of a biomarker of the invention comprise materials and reagents that are necessary for measuring the expression of the RNA product. For example, a microarray or RT-PCR kit may be used and contain only those reagents and materials necessary for measuring the levels of RNA products of one or more, or all or any combination of the biomarkers of the invention. Alternatively, in some embodiments, the kits can comprise materials and reagents that are not limited to SEQ ID NOs 1-6, or all, or any combination thereof. For example, a microarray kit may contain reagents and materials necessary for measuring the levels of RNA products of one or more of SEQ ID NOs 1-6. In a specific embodiment, a microarray or RT-PCR kit contains reagents and materials necessary for measuring the levels of RNA products of one of at least 1, at least 2, at least 3, at least 4, at least 5, or at least 6, or all or any combination of the biomarkers of the invention.

For nucleic acid micoarray kits, the kits generally comprise probes attached to a solid support surface. The probes may be labeled with a detectable label. In a specific embodiment, the probes are specific for the 5′ region, the 3′ region, the internal coding region, an exon(s), an intron(s), an exon junction(s), or an exon-intron junction(s), of 1, or more, or all or any combination of the biomarkers of the invention. The microarray kits may comprise instructions for performing the assay and methods for interpreting and analyzing the data resulting from the performance of the assay. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

For RT-PCR kits, the kits generally comprise pre-selected primers specific for particular RNA products (e.g., an exon(s), an intron(s), an exon junction(s), and an exon-intron junction(s)) of one or more, or all or any combination of the biomarkers of the invention. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for one or more, or all or any combination of the biomarkers of the invention. The probes may or may not be labeled with a detectable label (e.g., a fluorescent label). Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting and analyzing the data resulting from the performance of the assay.

For antibody based kits, the kit can comprise, for example: (1) a first antibody (which may or may not be attached to a solid support) which binds to protein of interest (e.g., a protein product of one or more, or all or any combination of the biomarkers of the invention); and, optionally, (2) a second, different antibody which binds to either the protein, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

Reporter gene-based assays may also be conducted to identify a compound to be tested for an ability to prevent, treat, manage or ameliorate osteoarthritis or a symptom thereof. In a specific embodiment, the present invention provides a method for identifying a compound to be tested for an ability to prevent, treat, manage or ameliorate osteoarthritis or a symptom thereof, said method comprising: (a) contacting a compound with a cell expressing a reporter gene construct comprising a reporter gene operably linked to a regulatory element of a biomarker of the invention (e.g., a promoter/enhancer element); (b) measuring the expression of said reporter gene; and (c) comparing the amount in (a) to that present in a corresponding control cell that has not been contacted with the test compound, so that if the amount of expressed reporter gene is altered relative to the amount in the control cell, a compound to be tested for an ability to prevent, treat, manage or ameliorate osteoarthritis or a symptom thereof is identified. In accordance with this embodiment, the cell may naturally express the biomarker or be engineered to express the biomarker. In another embodiment, the present invention provides a method for identifying a compound to be tested for an ability to prevent, treat, manage or ameliorate osteoarthritis or a symptom thereof, said method comprising: (a) contacting a compound with a cell-free extract and a reporter gene construct comprising a reporter gene operably linked to a regulatory element of a biomarker of the invention (e.g., a promoter/enhancer element); (b) measuring the expression of said reporter gene; and (c) comparing the amount in (a) to that present in a corresponding control that has not been contacted with the test compound, so that if the amount of expressed reporter gene is altered relative to the amount in the control, a compound to be tested for an ability to prevent, treat, manage or ameliorate osteoarthritis or a symptom thereof is identified.

Any reporter gene well-known to one of skill in the art may be used in reporter gene constructs used in accordance with the methods of the invention. Reporter genes refer to a nucleotide sequence encoding a RNA transcript or protein that is readily detectable either by its presence (by, e.g., RT-PCR, Northern blot, Western Blot, ELISA, etc.) or activity. Reporter genes may be obtained and the nucleotide sequence of the elements determined by any method well-known to one of skill in the art. The nucleotide sequence of a reporter gene can be obtained, e.g., from the literature or a database such as GenBank. Alternatively, a polynucleotide encoding a reporter gene may be generated from nucleic acid from a suitable source. If a clone containing a nucleic acid encoding a particular reporter gene is not available, but the sequence of the reporter gene is known, a nucleic acid encoding the reporter gene may be chemically synthesized or obtained from a suitable source (e.g., a cDNA library, or a cDNA library generated from, or nucleic acid, preferably poly A+RNA, isolated from, any tissue or cells expressing the reporter gene) by PCR amplification. Once the nucleotide sequence of a reporter gene is determined, the nucleotide sequence of the reporter gene may be manipulated using methods well-known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate reporter genes having a different amino acid sequence, for example to create amino acid substitutions, deletions, and/or insertions.

### Selecting Compounds or Agents

The present invention further provides a method of discovery of agents or compounds which modulate F-spondin expression. Compounds so identified can then be tested in appropriate in vitro or in vivo (animal) models of arthritis to determine whether they may be useful for modulating OA or related cartilage degenerative conditions. These compounds may also be assessed for modulation of endochondral bone formation and osteogenesis or for stimulation of bone growth in the epiphyseal growth plates of long bones. Thus, in one embodiment, methods are provided for screening agents or compounds which modulate F-spondin expression or function. In addition to the level of expression of F-spondin being elevated in patients suffering from osteoarthritis or a cartilage degenerative condition, the location of F-spondin in the extracellular matrix (ECM) is also important. Based on structure, normally F-spondin is associated with ECM, under pathological conditions, F-spondin may be released from ECM by the actions of plasmin and protease which causes F-spondin to have influence on the local metabolic activities.

In one embodiment, agents that modulate the expression or function of F-spondin are identified in a cell-based assay system. In accordance with this embodiment, cells expressing F-spondin, or a fragment thereof, are contacted with a candidate compound or a control compound and the ability of the candidate compound to alter the expression or function of F-spondin is determined. If desired, this assay may be used to screen a plurality (e.g. a library) of candidate compounds. The cell, for example, can be of prokaryotic origin (e.g., E. coli) or eukaryotic origin (e.g., yeast, insect or mammalian). Further, the cells can express F-spondin endogenously or be genetically engineered to express F-spondin, or a fragment or a fusion protein. In some embodiments, F-spondin, or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between F-spondin and a candidate compound. The ability of the candidate compound to interact directly or indirectly with F-spondin or a fragment thereof or a fusion protein or to modulate the expression of activity/function of F-spondin can be determined by methods known to those of skill in the art. For example, the interaction or modulation by a candidate compound can be determined by flow cytometry, a scintillation assay, immunoprecipitation or western blot analysis, based on the present description.

Another method includes the exposure of a chondrocyte cell culture to a candidate compound, and determining the duration and intensity of the response (for instance, the expression and/or function of the F-spondin) in the presence of the candidate compound and comparing the duration and intensity to that response in the absence of the candidate compound or in the presence of a known F-spondin inhibitor, such as an antibody. The comparison step of the invention can be preferably performed directly, i.e., by comparing the culture's response to the candidate F-spondin modulator to that of a known F-spondin modulator in a contemporaneous parallel culture. Alternatively, the comparison can be made with a historical control showing an effect on F-spondin expression and/or function that is comparable to that observed under the same conditions with the culture and a known F-spondin modulator.

In an alternative embodiment, the comparison is performed longitudinally. Replicate cultures, i.e., at least duplicate, are established and the candidate compound is introduced into the cultures. The response of the cultures at time points that are shortly after the introduction and before and at or after some time (for instance one hour) following the introduction is determined. An F-spondin modulator can be identified by the persistence of the response by comparison to a contemporaneous control.

The test/candidate compounds may first be chosen based on their structural and functional characteristics, using one of a number of approaches known in the art. For instance, homology modeling can be used to screen small molecule libraries in order to determine which molecules would be candidates to interact with F-spondin thereby selecting plausible targets. The compounds to be screened can include both natural and synthetic compounds. Furthermore, any desired compound may be examined for its ability to interact with F-spondin including as described below.

The present invention demonstrates the use of anti-F-spondin antibodies for inhibiting F-spondin activity. For example, in an organ culture system, F-spondin inhibited tibial growth and blocking F-spondin with an ant-FS antibody led to an increase in limb growth. In addition, using alkaline phosphatase as a quantitative measure of chondrocyte maturation, F-spondin TSR domain inhibition by transient transfection of cDNA constructs or coculture with TSR domain specific F-spondin antibodies were evaluated for their effects on chondrocyte maturation. Blocking endogenous F-spondin activity with TSR domain specific antibodies resulted in inhibition of AP activity.

### Candidate Compounds and Agents

Examples of agents, candidate compounds or test compounds identified by the methods of the present invention for treating or preventing osteoarthritis and other cartilage degenerative conditions, or fibrosing conditions, such as, but not limited to, scleroderma, pulmonary fibrosis and retroperitoneal fibrosis, include, but are not limited to, nucleic acids (e.g., DNA and RNA), carbohydrates, lipids, proteins, peptides, peptidomimetics, small molecules and other drugs. These agents may also be utilized in situations or under conditions wherein cartilage is replaced by bone or bone growth is warranted or necessary. Modulation of F-spondin may be used in the modulation, alleviation or treatment of diseases of the transient growth cartilage of the long bones, including chondrodysplasias and dwarfism, on in bone diseases, bone degeneration, bone fractures or bone cancer where replacement of bone or endochondral bone formation is warranted or helpful.

In one preferred aspect, agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683). In one particular embodiment, a candidate compound/agent may be an inducer of F-spondin expression/function in cartilage/chondrocytes and may be selected from the group consisting of prostaglandin E2 (PGE2), a cAMP inducer, Bone morphogenic protein 2 (BMP-2), Insulin-like growth factor (IGF), Fibroblast growth factor basic (FGFbasic) and Transforming Growth factor b1 (TGF-b1). These agents were shown to induce F-spondin expression in human chondrocytes as analyzed by TaqMan quantitative PCR and thus may be effective in a clinical setting, as described herein. Accordingly, it is envisioned that these agents may be used for treating conditions as described herein.

Phage display libraries may be used to screen potential F-spondin modulators. Their usefulness lies in the ability to screen, for example, a library displaying a billion different compounds with only a modest investment of time, money, and resources. For use of phage display libraries in a screening process, see, for instance, Kay et al., Methods, 240-246, 2001. An exemplary scheme for using phage display libraries to identify compounds that bind or interact with F-spondin, or alter the expression and/or function of F-spondin may be described as follows: initially, an aliquot of the library is introduced into microtiter plate wells that have previously been coated with target protein, e.g. F-spondin. After incubation (e.g. 2 hrs), the nonbinding phage are washed away, and the bound phage are recovered by denaturing or destroying the target with exposure to harsh conditions such as, for instance pH 2, but leaving the phage intact. After transferring the phage to another tube, the conditions are neutralized, followed by infection of bacteria with the phage and production of more phage particles. The amplified phage are then rescreened to complete one cycle of affinity selection. After three or more rounds of screening, the phage are plated out such that there are individual plaques that can be further analyzed. For example, the conformation of binding activity of affinity-purified phage for F-spondin may be obtained by performing ELISAs. One skilled in the art can easily perform these experiments. In one aspect, an F-spondin molecule used for any of the assays may be selected from a recombinant F-spondin protein, or an F-spondin fusion protein, an analog, derivative, or mimic thereof.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

The methods of screening compounds may also include the specific identification or characterization of such compounds, whose chondrocyte differentiating potential or bone growth potential was determined by the methods described herein. If the identity of the compound is known from the start of the experiment, no additional assays are needed to determine its identity. However, if the screening for compounds that modulate F-spondin is done with a library of compounds, it may be necessary to perform additional tests to positively identify a compound that satisfies all required conditions of the screening process. There are multiple ways to determine the identity of the compound. One process involves mass spectrometry, for which various methods are available and known to the skilled artisan.

Antibodies, including polyclonal and monoclonal antibodies, particularly anti-F-spondin antibodies may be useful as compounds to modulate chondrocyte differentiation and/or function. These antibodies are available from GenWay (15-288-22651), which is a chicken anti-spondin antibody; from Novus Biologicals (H00010418-M01), which is a mouse anti-human F-spondin clone 3F4; and GeneTex (GTX14271), which is a chicken anti-spondin 1 antibody, or they made be prepared using standard procedures for preparation of polyclonal or monoclonal antibodies known to those skilled in the art. Also, antibodies including both polyclonal and monoclonal antibodies, and drugs that modulate the activity of F-spondin may possess certain diagnostic applications and may for example, be utilized for the purpose of detecting and/or measuring conditions such as OA and related cartilage degenerative conditions and/or chondrocyte function or chondrocyte differentiation. F-spondin or fragments thereof may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of F-spondin may be discovered or synthesized, and may be used in diagnostic and/or therapeutic protocols.

Antisense and siRNA

Antisense oligonucleotides, ribozyme, and small interfering RNAs may be used to interfere with the expression of F-spondin or active fragments thereof at the translational level. The DNA sequences described herein may thus be used to prepare antisense molecules against, ribozymes and small interfering RNAs that cleave mRNAs or facilitate the degradation of mRNAs for F-spondin, active fragments of F-spondin. This approach can utilize antisense nucleic acid and ribozymes to block translation of a specific mRNA, either by masking that mRNA with an antisense nucleic acid or cleaving it with a ribozyme. Antisense nucleic acids are DNA or RNA molecules that are complementary to at least a portion of a specific mRNA molecule. (See Weintraub, 1990; Marcus-Sekura, 1988.) In the cell, they hybridize to that mRNA, forming a double stranded molecule. The cell does not translate an mRNA in this double-stranded form. Therefore, antisense nucleic acids interfere with the expression of mRNA into protein. The antisense or oligonucleotide may be modified to enhance nuclease resistance. Nucleic acids which contain at least one phosphorothioate modification are particularly preferred (Geary, R. S. et al (1997) Anticancer Drug Des 12:383-93; Henry, S. P. et al (1997) Anticancer Drug Des 12:395-408; Banerjee, D. (2001) Curr Opin Investig Drugs 2:574-80). Specific examples of some preferred oligonucleotides envisioned include those containing modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones. The amide backbones disclosed by De Mesmaeker et al. (1995) Acc. Chem. Res. 28:366-374) are also preferred. Also preferred are oligonucleotides having morpholino backbone structures (Summerton and Weller, U.S. Pat. No. 5,034,506). In other particular embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al., Science, 1991, 254, 1497). Nucleic acids may also contain one or more substituted sugar moieties. Antisense or oligonucleotides may comprise one of the following at the 2′ position: OH, SH, SCH₃, F, OCN, heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. Similar modifications may also be made at other positions on the oligonucleotide, particularly the 3′ position of the sugar on the 3′ terminal nucleotide and the 5′ position of 5′ terminal nucleotide.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, 1988.). Because they are sequence-specific, only mRNAs with particular sequences are inactivated. Investigators have identified two types of ribozymes, Tetrahymena-type and “hammerhead”-type. (Hasselhoff and Gerlach, 1988) Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The use of RNA interference strategies to inhibit the expression of F-spondin or active fragments thereof is further embodied in the invention. Thus, methods of RNA interference and small interfering RNA compositions are included in the methods and compositions of the present invention. RNA interference refers to the silencing of genes specifically by double stranded RNA (dsRNA) (Fine, A. et al (1998) Nature 391; 806-811). In one embodiment, short or small interfering RNA (siRNA) is utilized (Elbashir, S. M. et al (2001) Nature 411:494-498). In addition, long double stranded RNA hairpins may be employed (Tavernarakis, N. et al (2000) Nature Genet. 24:180-183; Chuang, C. F. and Meyerowitz, E. M. (2000) PNAS USA 97:4985-90; Smith, N A et al (2000) Nature 407:319-20). F-spondin siRNA, shRNA and lentiviral particles can be generated and tested by one of skill in the art and products are available commercially, including from Santa Cruz Biotechnology, Inc, CA (sc-60613) and Sigma Aldrich, St. Louis, Mo. Human Spon1 (Cat. No: SASI_Hs02_{—}00340896-902) and mouse Spon1 (SASI_Mm01_{—}00109024-9031). Human and Mouse Spon1 shRNA constructs are from Sigma Aldrich, St. Louis, Mo. Exemplary shRNAs for human F-spondin include:

(1)TRCN0000116979 NM_006108.1-827s1c1 TRC 1;
Region: CDS Alternate Species: NM_006108.2;
(SEQ ID NO: 47)
Sequence: CCGGGCCAAGTACAGACTCACATTTCTCGAGAAATGTGAGTCTGTACTTGGCTTT
TTG
(2)TRCN0000116981 NM_006108.1-1417s1c1 TRC 1;
Region: CDS Alternate Species: NM_006108.2
(SEQ ID NO: 48)
Sequence: CCGGCAGAGTTGTCATCGAGAGAATCTCGAGATTCTCTCGATGACAACTCTGTTT
TTG
(3)TRCN0000116980 NM_006108.1-2147s1c1 TRC 1;
Region: CDS Alternate Species: NM_006108.2
(SEQ ID NO: 49)
Sequence: CCGGGCAGAACTTGGAGACTGCAATCTCGAGATTGCAGTCTCCAAGTTCTGCTTT
TTG
(4)TRCN0000116978 NM_006108.1-1469s1c1 TRC 1;
Region: CDS Alternate Species: NM_006108.2
(SEQ ID NO: 50)
Sequence: CCGGCCTGACAATGTCGATGATATTCTCGAGAATATCATCGACATTGTCAGGTTT
TTG
(5)TRCN0000116977 NM_006108.1-2690s1c1 TRC 1;
Region: 3 UTR Alternate Species: NM_006108.2
(SEQ ID NO: 51)
Sequence: CCGGGCTGGATTATTTGCTTGTTTACTCGAGTAAACAAGCAAATAATCCAGCTTT
TTG

Exemplary shRNAs for mouse F-spondin include:

(1)TRCN0000090522 NM_145584.1-1284s1c1 TRC 1;
Region: CDS Alternate Species: NM_145584.1
(SEQ ID NO: 52)
Sequence: CCGGGACCTATGAGTCACCAAACAACTCGAGTTGTTTGGTGACTCATAGGTCTTT
TTG
(2)TRCN0000090521 NM_145584.1-2529s1c1 TRC 1;
Region: CDS Alternate Species: NM_145584.1
(SEQ ID NO: 53)
Sequence: CCGGGCGCTACATGACTGTGAAGAACTCGAGTTCTTCACAGTCATGTAGCGCTTT
TTG
(3)TRCN0000090520 NM_145584.1-814s1c1 TRC 1;
Region: CDS Alternate Species: NM_145584.1
(SEQ ID NO: 54)
Sequence: CCGGGCCAAGTACAGACTCACGTTTCTCGAGAAACGTGAGTCTGTACTTGGCTTT
TTG
(4)TRCN0000090519 NM_145584.1-1281s1c1 TRC 1;
Region: CDS Alternate Species: NM_145584.1
(SEQ ID NO: 55)
Sequence: CCGGCGTGACCTATGAGTCACCAAACTCGAGTTTGGTGACTCATAGGTCACGTTT
TTG
(5)TRCN0000090518 NM_145584.1-3218s1c1 TRC 1;
Region: 3UTR Alternate Species: NM_145584.1
(SEQ ID NO: 56)
Sequence: CCGGGCAGGTGATGATGGCTACTTTCTCGAGAAAGTAGCCATCATCACCTGCTTT
TTG

### Methods of Treatment and Therapeutic and Prophylactic Compositions and Use

Candidates for therapy with the agents identified by the methods described herein are patients either suffering from an arthritic condition, such as osteoarthritis, or other cartilage degenerative conditions. In addition, it is envisioned that agents identified by the methods disclosed herein may also be useful for treating fibrosing disorders, such as, but not limited to scleroderma, pulmonary fibrosis and retroperitoneal fibrosis. Also, since F-spondin is expressed in growth plate cartilage and enhances chondrocyte maturation, modulation of F-spondin is proposed for enhancing cartilage repair and preventing or treating cartilage degeneration. Thus, administration or stimulation of F-spondin or of F-spondin's effects can mediate cartilage repair and/or reduce cartilage degeneration. F-spondin may therefore be administered, a fragment thereof, nucleic acids encoding F-spondin or an active fragment thereof may be utilized, or an agent which modulates expression or activity of F-spondin may be used in enhancing cartilage repair, preventing cartilage degeneration or an arthritic condition, or treating an arthritic condition. The agents may be utilized in situations or under conditions wherein cartilage is replaced by bone or bone growth is warranted or necessary. Modulation of F-spondin may be used in the modulation, alleviation or treatment of diseases of the transient growth cartilage of the long bones, including chondrodysplasias and dwarfism, on in bone diseases, bone degeneration, bone fractures or bone cancer where replacement of bone or endochondral bone formation is warranted or helpful.

The invention provides methods of treatment comprising administering to a subject an effective amount of F-spondin, an active fragment thereof or an agent of the invention. In a preferred aspect, the compound is substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the agents identified by the methods described herein may be formulated as pharmaceutical compositions to be used for prophylaxis or therapeutic use to treat these patients.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled or sustained release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

In a preferred embodiment of the invention, a method of treating OA or a related cartilage degenerative condition, or a fibrosing disorder is envisioned by administering a compound or agent that normalizes the levels of F-spondin in a subject.

The present invention further contemplates therapeutic compositions useful in practicing the therapeutic methods of this invention. A subject therapeutic composition includes, in admixture, a pharmaceutically acceptable excipient (carrier) and one or more F-spondin modulators, as described herein as an active ingredient. In a preferred embodiment, the composition comprises one or more compounds or agents capable of normalizing the levels of F-spondin in cells or tissues.

Effects of the compounds or agents of the invention can first be tested for their ability to modulate expression levels (up or down-regulate) or one or more functions of F-spondin. More particularly, the selectivity of the compounds for F-spondin can be assessed using any of the methods described herein. Cells can be transfected with the nucleic acid encoding F-spondin and assays done to determine the effect of a compound on F-spondin expression levels or activity/function.

Modulators of F-spondin may be selected from the group consisting of prostaglandin E2 (PGE2), cAMP inducers, Bone morphogenic protein 2 (BMP-2), Insulin-like growth factor (IGF), Fibroblast growth factor basic (FGFbasic) and Transforming Growth factor b1 (TGF-b1). These agents induced F-spondin expression in human chondrocytes as analyzed by TaqMan quantitative PCR. Accordingly, it is envisioned that these agents may be used for treating conditions as described herein.

Further confirmation of the activity of the compounds can be tested in relevant in vivo models. Examples of animal models for studying arthritis and cartilage degenerative conditions may be found in the following publications, which are incorporated in their entireties: Ameye, L. G. et al., Current Opinion in Rheumatology (2006), 18(5):537-547; Warskyj, M. and Hukins, D W, Br. J. of Rheumatology (1990), 29:219-221; Botter, S. M. et al., Biorheology, (2006), 43(3-4):379-388; Carlson, C. S. et al., J. Bone Miner. Res. (1996), September; 11(9):1209-1217; Moreau, M. et al., J. Rheumatology (2006), June; 33(6):1176-1183; Laurent, D. et al. Skeletal Radiol., (2006), August; 35(8):555-564; Hotta, H. et al. J. Orthop. Sci. (2005), November; 10(6):595-607; Mastbergen, S. C. et al. Rheumatology (Oxford), (2006), April; 45(4):405-413; and Mastbergen, S. C. et al., Osteoarthritis Cartilage (2006), January; 14(1):39-46. Chambers M. G. et al., Arthritis and Rheumatism (2001) June 44(6):1455-1465.

The present compounds or agents that modulate F-spondin expression or function can be used as the sole active agents, or can be used in combination with one or more other active ingredients. In particular, combination therapy using the F-spondin modulators with one or more other agents that have an effect in treating OA or related cartilage degenerative conditions or fibrosing disorders are contemplated. These agents are known in the art, and can be selected from non-steroidal anti-inflammatory compounds, analgesics, or other compounds useful in enhancing bone turnover, including an antiresorptive drug, a bone-forming agent, an estrogen receptor antagonist and a drug that has a stimulatory effect on osteoclasts. More particularly, the antiresorptive drug may be selected a bisphosphonate, an estrogen or estrogen analogue, a selective estrogen receptor modulator (SERM) and a calcium source, Tibolone, calcitonin, a calcitriol and hormone replacement therapy. The bone-forming agent may be selected from parathyroid hormone (PTH) or a peptide fragment thereof, PTH-related protein (PTHrp), bone morphogenetic protein, osteogenin, NaF, a PGE₂ agonist, a statin, and a RANK ligand (RANKL). The drug that has a stimulatory effect on osteoclasts may be vitamin D, or a vitamin D derivative or mimic thereof. The estrogen receptor antagonist may be raloxifene, bazedoxifene and lasofoxifene. The bisphosphonate may be alendronate, risedronate, ibandronate and zoledronate. Compositions comprising one or more F-spondin modulators and one or more other antiresorptive or anabolic agents are provided and included in the invention.

When contemplating combination therapy with an F-spondin modulator and one or more of the above-noted agents, it is important to assess clinical safety by methods known to those skilled in the art. Appropriate dose titration may be necessary when certain groups of compounds are contemplated for use together.

The compounds or compositions of the invention may be combined for administration with or embedded in polymeric carrier(s), biodegradable or biomimetic matrices or in a scaffold. The carrier, matrix or scaffold may be of any material that will allow composition to be incorporated and expressed and will be compatible with the addition of cells or in the presence of cells. Preferably, the carrier matrix or scaffold is predominantly non-immunogenic and is biodegradable. Examples of biodegradable materials include, but are not limited to, polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to administration or implantation, e.g., by treatment with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof. Matrices suitable include a polymeric mesh or sponge and a polymeric hydrogel. In the preferred embodiment, the matrix is biodegradable over a time period of less than a year, more preferably less than six months, most preferably over two to ten weeks. The polymer composition, as well as method of manufacture, can be used to determine the rate of degradation. For example, mixing increasing amounts of polylactic acid with polyglycolic acid decreases the degradation time. Meshes of polyglycolic acid that can be used can be obtained commercially, for instance, from surgical supply companies (e.g., Ethicon, N.J). A hydrogel is defined as a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions, that have charged side groups, or a monovalent ionic salt thereof.

For use in treatment of animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The preparation of therapeutic compositions which contain small organic molecules polypeptides, analogs or active fragments as active ingredients is well understood in the art. The compositions of the present invention may be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, or intravenously. Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, nasal, or oral administration. Such compositions may be prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. The formulation will generally include a diluent as well as, in some cases, adjuvants, buffers, preservatives and the like. In addition, if desired, the composition can contain minor amounts of auxiliary substances such as wetting or emulsifying agents, pH buffering agents which enhance the effectiveness of the active ingredient.

A small organic molecule/compound, a polypeptide, an analog or active fragment thereof can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. For oral administration, the compositions can be administered also in liposomal compositions or as microemulsions. Suitable forms include syrups, capsules, tablets, as is understood in the art.

The compositions of the present invention may also be administered locally to sites in subjects, both human and other vertebrates, such as domestic animals, rodents and livestock, where bone formation and growth are desired using a variety of techniques known to those skilled in the art. For example, these may include sprays, lotions, gels or other vehicles such as alcohols, polyglycols, esters, oils and silicones. Such local applications include, for example, into the arthritic joint.

The administration of the compositions of the present invention may be pharmacokinetically and pharmacodynamically controlled by calibrating various parameters of administration, including the frequency, dosage, duration mode and route of administration. Thus, in one embodiment bone mass formation is achieved by administering a bone forming composition in a non-continuous, intermittent manner, such as by daily injection and/or ingestion. Variations in the dosage, duration and mode of administration may also be manipulated to produce the activity required.

The therapeutic F-spondin modulator compositions are conventionally administered in the form of a unit dose, for instance intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the agent selected for treating the subject, the dosage formulation, and in a therapeutically effective amount. The phrase “in an amount sufficient to modulate F-spondin expression or function” refers to the amount of an F-spondin modulator necessary to achieve localized (at the site of injury or diseased tissue or cells) concentrations of the modulator, ranging from about 0.01 nM to about 100 mM, more preferably about 0.1 nM to about 1 mM, and most preferably from about 1 nM to about 1 μM, to provide the desired effect. The desired effect refers to the effect of the agent on amelioration of at least one symptom, such as, pain or swelling associated with the arthritic or fibrosing condition, using the methods as described herein, or a slowing of disease progression. Moreover, the quantity of the F-spondin modulator to be administered depends on the subject to be treated, and degree of or the extent or severity of the disease. Precise amounts of active ingredient required to be administered depend on the judgment of the practitioner and are peculiar to each individual. However, suitable dosages to achieve the desired therapeutic effect may range from about 0.01 to 100 mg/kg body weight, preferably about 0.01 to 10, preferably about 0.01 to 0.1, preferably about 0.01 to 0.5, preferably about 0.1 to 0.5, preferably about 0.5 to about 10, and more preferably one to several, milligrams of active ingredient per kilogram body weight of individual per day and depend on the route of administration. However, dosage levels are highly dependent on the nature of the disease or situation, the condition of the subject, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary. Suitable regimes for initial administration and further administration are also variable, but are typified by an initial administration followed by repeated doses at one or more hour intervals by a subsequent injection or other administration. Alternatively, continuous intravenous infusion sufficient to maintain desired concentrations, e.g. in the blood, are contemplated. The composition may be administered as a single dose multiple doses or over an established period of time in an infusion.

It will be understood that the appropriate dosage of the substance should suitably be assessed by performing animal model tests, wherein the effective dose level (e.g. ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

The compound or composition of the present invention may be modified or formulated for administration at the site of disease. Such modification may include, for instance, formulation which facilitate or prolong the half-life of the compound or composition, particularly in the local environment. Additionally, such modification may include the formulation of a compound or composition to include a targeting protein or sequence which facilitates or enhances the uptake of the compound/composition to cartilage. In a particular embodiment, such modification results in the preferential targeting of the compound to cartilage or the arthritic joint versus other locations or cells.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

The pharmaceutical compositions of this invention may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Topical application can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference, 54^{th} Edition (2000).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Increased Expression of F-Spondin in Osteoarthritis

### A. Global Gene Expression Studies in OA Cartilage in Human Subjects

Genomic studies designed to identify novel differentially expressed genes in osteoarthritis were performed. Total RNA was isolated from 20 non-arthritic and 50 osteoarthritic cartilages and pooled. Five OA pools and two normal pools, each comprised of ten individual patient RNA samples (1 ug each) were analyzed by microarray analysis according to an Affymetrix protocol. The data was further normalized and analyzed using a dchip program. Over 200 genes, including F-spondin, were determined to be significantly upregulated in OA versus normal as determined by hierarchical clustering (Attur M G, Dave M N, Tsunoyama K, Akamatsu M, Kobori M, Miki J, et al. “A system biology” approach to bioinformatics and functional genomics in complex human diseases: arthritis. Curr Issues Mol Biol 2002; 4(4):129-46), as shown in FIG. 1a and b) and confirmed by quantitative polymerase chain reaction (QPCR) in 18 individual OA cartilage specimens as shown in FIG. 1C. The expression pattern of F-spondin in other normal tissues and carcinomas was further analyzed using the SOURCE database (http://source.stanford.edu), which indicated that F-spondin is highly expressed in brain, lung and intestine as compared to other tissues (data not shown). However, F-spondin has not been reported in chondrocytes, as revealed by our studies.

### B. Western Analysis of F-Spondin in OA Cartilage

Western blot analyses was performed in order to assess F-spondin protein expression in OA cartilage. Total protein from 4 normal and 4 osteoarthritic cartilage samples was extracted (Amin A R, Attur M, Patel R N, Thakker G D, Marshall P J, Rediske J, et al. Superinduction of cyclooxygenase-2 activity in human osteoarthritis-affected cartilage. Influence of nitric oxide. J Clin Invest 1997; 99(6):1231-7) and resolved using 10% SDS-PAGE and probed with rabbit polyclonal antiserum to human F-spondin (kindly provided by Dr. Klar) (FIG. 2); catalase was used as an internal control for protein loading. OA samples showed elevated expression of F-spondin as compared to normal cartilage.

### C. Immunodetection of F-Spondin in OA Cartilage

The expression of F-spondin was also confirmed by immunohistochemistry in lesional and non-lesional OA cartilage obtained at the time of surgery (FIG. 3). Immunostaining demonstrates intense staining of F-spondin in superficial zone associated with chondrocytes and matrix. In non-lesional cartilage, immunostaining of F-spondin was similar but also observed in the middle zone. The F-spondin distribution was comparable to type II collagen in non-lesional cartilage.

D. F-Spondin mRNA is Upregulated in a Surgical Rat Model of OA

Gene microarray analysis was utilized to study gene expression in the rat ACL and partial medial meniscectomy (ACL/PM) model of osteoarthritis. Rats were analyzed four weeks after surgery, at the onset of histologically confirmed osteoarthritis. RNA samples were obtained from the articular cartilage of sham (control), ipsilateral (operated) OA, and contralateral knees and hybridized to Affymetrix RAE230_{—}2.0 GeneChips®. As shown in FIG. 4, F-spondin gene expression increased 7-fold in the operated knee, and was among the most highly expressed genes in rat OA cartilage. Surprisingly, a moderate increase in F-spondin is also observed in contralateral knee, which may be due to increased mechanical strain. This suggests that F-spondin could be followed as a potential early OA biomarker.

### Example 2

### F-Spondin in Chondrogenesis

### A. Distribution of F-Spondin in the Chick Embryo Growth Plate

Because of its known role in neuronal development, studies were performed to determine whether F-spondin was expressed in chick embryo growth plate chondrogenesis. Recently, it has been shown (FIG. 5a) that expression of eNOS and the generation of NO enhanced maturation of chondrocytes occurs by upregulating alkaline phosphatase and collagen type X expression (Teixeira C C, Ischiropoulos H, Leboy P S, Adams S L, Shapiro I M. Nitric oxide-nitric oxide synthase regulates key maturational events during chondrocyte terminal differentiation. Bone 2005; 37(1):37-45). Eventually the chondrocytes in the center of this model undergo maturation, becoming hypertrophic and finally undergoing apoptosis. During hypertrophy cells express various hypertrophic markers such as increased plasma membrane alkaline phosphatase activity, elevated synthesis of type X collagen, down regulation of type II collagen production, enhanced secretion of osteonectin, and osteocalcin. In the epiphysial growth plate, chondrocytes form well defined morphologic zones: resting, proliferative, hypertrophic and calcified cartilage regions (FIG. 5b). As shown in FIG. 5b, F-spondin is expressed in the growth plate, and its expression is most prominent in hypertrophic and calcified zone. We will utilize the chick embryo growth plate model to assess a potential role for F-spondin in chondrocyte maturation and the development of the hypertrophic phenotype.

### B. Postnatal Mesenchymal Stem Cells

When cultured as high density aggregates, postnatal mesenchymal stem cells also undergo chondrogenesis (Johnstone B, Hering T M, Caplan A I, Goldberg V M, Yoo J U. In vitro chondrogenesis of bone marrow-derived mesenchymal progenitor cells. Exp Cell Res 1998; 238(1):265-72). Following exposure to chondrogenic growth factors, cultures exhibit metachromatic staining with toluidine blue and corresponding immunostaining for type II collagen, characteristic of cartilage extracellular matrix. In preliminary studies, we have found that adult bone marrow-derived MSCs undergo chondrogenesis in aggregate cultures following exposure to TGF-β 1 and BMP-2 in agreement with the findings of others

(Johnstone B, Hering T M, Caplan A I, Goldberg V M, Yoo J U. In vitro chondrogenesis of bone marrow-derived mesenchymal progenitor cells. Exp Cell Res 1998; 238(1):265-72; Barry F, Boynton R E, Liu B, Murphy J M. Chondrogenic differentiation of mesenchymal stem cells from bone marrow: differentiation-dependent gene expression of matrix components. Exp Cell Res 2001; 268(2):189-200; Palmer G D, Steinert A, Pascher A, Gouze E, Gouze J-N, Betz O, et al. Gene-Induced Chondrogenesis of Primary Mesenchymal Stem Cells in vitro. Molecular Therapy 2005; 12(2):219-228)

Upon histological examination important differences between TGF-β1 and BMP-2-treated cultures were noted (FIG. 6). BMP-2-treated cultures were typically larger, more cellular and showed more intense staining for proteoglycan, type II and type X collagen, whereas staining in TGF-β 1-treated aggregates was generally lower. These findings suggest that TGF-β 1 and BMP-2 modulate chondrocyte differentiation to differing extents. We propose to extend these studies further, in order to more fully characterize extracellular matrix protein expression during chondrocyte maturation and the relationship to F-spondin, by comparing the effects of various chondroinductive factors.

### Example 3

### F-Spondin and Chondrocyte Functions

Having demonstrated increased gene and protein expression of F-spondin in OA cartilage, studies were then done to characterize its function. Preliminary data suggest that F-spondin expression is regulated by IL-1 β and anabolic growth factors similar to other ECM matrix proteins, type II collagen and aggrecan. In addition, as reported below, F-spondin exerts significant effects on chondrocyte function, most notable for capacity to inhibit IL-1 β and TNFα expression as well as stimulation of anabolic growth factors such as TGF-β 1, BMP-2 and ECM proteins, type II collagen and aggrecan.

### A. Regulation of F-Spondin Expression: Effect of IL-1 β

Since IL-1 β appears to play a significant role in the pathogenesis of OA, its effects on F-spondin expression by QPCR in cartilage explants cultures was studied. Briefly, knee articular cartilage from patients undergoing knee replacement surgery was obtained and cut in 3-mm discs, and four to six discs (˜100 mg) were placed in organ culture in 2 ml of Ham's F-12 medium+0.1% human albumin (endo toxin fr) for 24-72 h, in the presence and absence of IL-1beta (1 ng/ml). At the end of the experiment the cartilage was frozen immediately in liquid nitrogen for RNA extraction. As shown in FIG. 7, unstimulated OA cartilage constitutively expressed F-spondin. Exposure to IL-1 (1 ng/ml) significantly inhibited the expression of F-spondin expression and aggrecan.

### B. Effect of Growth Factors on F-Spondin Expression in Human OA Chondrocytes

The expression of F-spondin in the presence of growth factors such as TGF-β1 or FGF was studied in human chondrocytes by QPCR (FIG. 8). Human chondrocytes were isolated from OA-affected cartilage. The cartilage was cut into small pieces and digested with Pronase (0.1%) for 30 min in phosphate-buffered saline, followed by digestion with collagenase P (0.1%) for 12-16 h in Ham's F-12 medium. This cell suspension was used to establish cell cultures and maintained at 37° C. in a humidified atmosphere of 95% air and 5% CO₂. The chondrocytes were maintained as monolayer cultures for not more than 3 days, to maintain the chondrocyte phenotype. The cells were serum starved for 24 h before stimulating with growth factors. The total RNA was isolated after 24 h post stimulation of cells using Qiagen Rneasy protocol. As shown, FGF basic, FGF-18 and TGF-β1 each enhanced the expression of F-spondin by 1.5 to 3 fold at 24 h. Similar increases were observed following exposure to retinol. These stimuli exerted comparable effects on the expression of type II collagen and aggrecan mRNA (not shown).

C. Effects of F-Spondin and its Cleavage Products on Chondrocyte Functions. Molecular Cloning and Domain Organization of F-Spondin

F-spondin is located on chromosome 11, the gene is 305 kb in size and the cDNA sequence is derived from 16 exons. We cloned the human full length F-spondin that is approximately 2.4 kb using RT-PCR and confirmed using di-deoxy sequencing. Computer analysis using Genelynx and Genecards revealed the presence of multiple domains of F-spondin with protein of molecular mass ˜80 kDa. Due to glycosylation, the protein migrates at 105 kDa. We also found that the antibody cross reacted with several small fragments ˜40-60 kDa. In order to do functional studies, various constructs were obtained representing different domains of F-spondin, as described in FIG. 9, for functional expression studies in a chondrocyte cell line, C28I2. In our initial screening of various chondrocyte cell lines, C28I2 expressed only 10-20 copies of F-spondin as assessed by TaqMan QPCR, and no protein was detected by western analysis. However, other cell lines including primary chondrocytes expressed F-spondin in the range of 3000-20,000 copies. For our initial functional studies, the following constructs were used: Ig.F-spondin.1 (FS1: full length), Ig.F-spondin.6 (FS6: representing only the reelin-like and spondin domain) and Ig.F-spondin.7 (FS7: representing only 1-6 TSR domains).

### D. Functional Expression Studies in C28I2 and Primary Chondrocytes

The following experimental strategy was used to study the role of F-spondin in primary chondrocytes. C28I2 cells were transfected with FS1, FS6 and FS7 and control vector by nucleofector reagent (Amaxa) and cells were grown in serum free medium for 24 h. The supernatants were collected and expression of F-spondin was confirmed using western blot analysis (not shown). C28I2 cells transfected with F-spondin were lysed with TRIzol and used for RNA isolation to study the effect of transgene expression of F-spondin on expression of various gene by TaqMan PCR.

### E. Genes Modulated by Transgene Expression of F-Spondin

The effect of transgene expression of F-spondin on various anabolic gene expression in the human chondrocyte cell line, C28I2, was studied. Full length F-spondin (FS1) induced expression of BMP-2, type II collagen, aggrecan and TGF-β1 (FIG. 10). Expression of FS6, which contains only reelin and spondin domains, in C28I2 cells, exhibited variable effects on the expression of the above genes, as compared to FS1. FS6 was less potent in inducing aggrecan, whereas, it markedly increased type II collagen (FIG. 10). FS7, which contains all TSP1-6 domains, was consistently less effective in inducing anabolic genes as compared to FS1 and FS6. Mindin, another non-thrombospondin family member or vector pCMV Ig were used as control. Mindin or control vector did not induce any of the anabolic genes studied.

### F. Effects of Exogenous F-Spondin on PGE2 Production by Chondrocytes

F-spondin consists of reelin, spondin and six TSR domains; these domains may activate various cellular functions by binding to different receptors. Previously, Terai et al (2001) have shown that F-spondin inhibits angiogenesis in endothelial cells by binding to αvβ3 (Terai Y, Abe M, Miyamoto K, Koike M, Yamasaki M, Ueda M, et al. Vascular smooth muscle cell growth-promoting factor/F-spondin inhibits angiogenesis via the blockade of integrin alphavbeta3 on vascular endothelial cells. J Cell Physiol 2001; 188(3):394-402). Blocking the effect of F-spondin using antibodies (R1, which recognizes the TSR domain 3-6 of F-spondin) (kind gift from Dr. Klar) specific for F-spondin or to αvβ3 (LM609; αvβ3 blocking Abs) would help us to identify the function of F-spondin in chondrocytes. Accordingly, human chondrocytes were incubated with the supernatant from F-spondin transfected cell culture for 24 h and the levels of PGE2 were measured under various conditions (FIG. 11). Addition of blocking antibody R1 or LM609, had no effect on spontaneous PGE2 production, however exposure to culture supernatants from F-spondin transfected cells significantly increased PGE2 production as compared to control vector transfected supernatants. F-spondin induced PGE2 production could be inhibited by anti-F-spondin blocking antibody R1 or integrin αvβ3 blocking Ab LM609. This study indicates that F-spondin increases PGE2 production via ligation of αvβ3.

### G. Effect of PGE2 on ECM Proteins and MMP Expression in Human Chondrocytes

F-spondin induced both catabolic and anabolic gene/gene products in chondrocytes including BMP-2, type II collagen, TGF-β1, COX-2, mPGES and PGE2. To begin to address the question of whether selected F-spondin effects in cartilage are mediated by prostaglandin production, we have begun studies to characterize the effect of PGE2 in cartilage. Addition of PGE2 (1-10 uM) to cartilage explants inhibited type II collagen, aggrecan expression, ³⁵SO4 incorporation, as well as increased type II collagen degradation as assessed by C12C ELISA (IBEX). PGE2, like F-spondin, also increased secretion of MMP-13, IL-6 and IL-8, but decreased MMP-1 secretion in chondrocytes (Attur M. Dave, M. Patel, J. Pillinger, M. Abramson, S. Prostaglandin E2 exerts catabolic effects in OA cartilage: evidence for signalling via the EP4 receptor. In: ACR; 2005; 2005. p. 1910). Our preliminary data indicate that these effects of PGE2 in cartilage are mediated via NURR1, an immediate early gene and member of the nuclear receptor superfamily. Using the identical clinical specimens (18 OA patients vs. 8 age-matched normal controls) in which we demonstrated increased expression of F-spondin reported above, we studied NURR1 mRNA expression using Affymetrix microarray. Relative to normals, NURR1 was overexpressed in OA cartilage (2-5 fold) and synovium (2 fold), confirmed by QPCR. Incubation of OA chondrocytes with PGE2 (1-10 uM) induced NURR1 expression (20-50 fold), as analyzed by QPCR. Since NURR1 binds to the NURR1 cis-acting sequence (NBRE) in the promoter region of a variety of genes, we examined the effect of adenoviral-mediated over-expression of NURR1 in chondrocytes. Chondrocytes transfected with NURR1 exhibited increased expression of mRNA for IL-6, IL-8 and MMP-13, and decreased expression of MMP-1 (Data not shown).

These effects were duplicated by addition of PGE2 (1-10 uM) to non-transfected chondrocytes. Strikingly, therefore, selected effects of the PGE2/cAMP/NURR1 pathway recapitulated selected effects of F-spondin, which we show increases PGE2 production in chondrocytes, as reported above. We therefore hypothesize that F-spondin exerts selected effects in cartilage via several distinctive signal transduction pathways, including those which result from its capacity to increase PGE2 production, the major eicosanoid product of articular cartilage (Amin A R, Attur M, Patel R N, Thakker G D, Marshall P J, Rediske J, et al. Superinduction of cyclooxygenase-2 activity in human osteoarthritis-affected cartilage. Influence of nitric oxide. J Clin Invest 1997; 99(6):1231-7). We will perform studies designed to elucidate prostaglandin-dependent and prostaglandin-independent effects of F-spondin in cartilage (as illustrated in FIG. 13).

### Example 4

### Potential Interaction Between F-Spondin and Other ECM Proteins

### A. Activation of Latent TGF-β1 by F-Spondin in OA Cartilage Explants

The effect of F-spondin on latent TGF-β1 activation in OA cartilage explant cultures was studied by ELISA (R&D systems). Unstimulated OA cartilage explants spontaneously released both latent TGF-β1 (˜11 ng/g cartilage) and active TGF-β1 (200 pg/g cartilage) in culture supernatants (FIG. 12). Addition of recombinant F-spondin (1 μg/ml) (R&D systems) increased the active TGF-β1 to 550 pg/g cartilage (by 225%) without significant changes in total latent TGF-β1 synthesis, as shown in FIG. 12.

### Example 5

### The Effects of F-Spondin and its Predicted Proteolytic Fragments on Chondrocyte Metabolism

Our preliminary observations demonstrated that F-spondin, originally described in neuronal tissue, but not previously observed in cartilage, is upregulated in human (and rat) osteoarthritis, where it exerts effects on key anabolic and catabolic chondrocyte functions (illustrated in FIG. 13). Studies will be done to characterize the specific cellular effects of F-spondin and the contributions to these effects of: 1) distinct properties of the intact molecule versus its proteolytic fragments, and 2) the activation of discrete signaling pathways (e.g., PGE2, TGF-β1) that amplify the biological actions of F-spondin in the extracellular matrix. The metabolic pathways regulated by F-spondin and its proteolytic fragments are characterized in order to elucidate its action in OA cartilage, a step toward the identification of novel target(s) for disease modification strategies.

Alginate cultures will be utilized, which provide a three-dimensional culture environment that allows the accumulation of synthesized extracellular matrix proteins, to assess the effects of F-spondin on chondrocyte cell responses in vitro. This and other methods, which will be used in this analysis are described below.

### Materials and Methods:

### Retroviral Vector Construction and Transduction

Full length F-spondin cDNA will be cloned into a modified version of a retroviral shuttle vector, pMSCVpuro (Clontech) containing an IRES GFP to enable positive selection of retroviral transfectants by fluorescence activated cell sorting (FACS). For vector construction, the F-spondin MIG-GFP shuttle vector will be cotransfected in 293T cells along with the retroviral helper plasmid, pCLEGO, encoding gag pol and env genes. Forty eight hours (48 h) after transfection, the supernatant will be collected, centrifuged briefly to remove cell debris and titrated for functional viral particles by infection of NIH3T3 cells (ATCC) in the presence of polybrene. Following confirmation of viable viral particles, viral supernatants will be used to infect human chondrocytes. At 72 h post infection, retrovirally transduced cells will be selected by FACS and expanded in culture prior to experiments. To generate vectors expressing truncated fragments of the F-spondin molecule, full-length F-spondin cDNA will be digested with restriction enzymes or exonuclease III so that expressed cDNAs correspond to predicted proteolytic fragments.

Knockdown of F-Spondin Gene Expression by siRNA

Short hairpin antisense siRNA probes targeting the 5′ upstream sequence of the F-spondin gene will be constructed using the Oligoengine software and synthesis program. Probes will be cloned into the pSUPERretroGFP retroviral vector shuttle plasmid (Oligoengine) and cotransfected with pCLEGO helper vector into 293T cells to generate MSCV retroviral particles (as described above). Gene knockdown will be confirmed by Western blot and RT-PCR of retrovirally infected chondrocyte monolayer cultures using F-spondin primary antibodies and primer sets, respectively.

### Alginate Cell Cultures

Following retroviral transduction and selection, human chondrocytes will be expanded in culture and seeded into filter-sterilized, low viscosity alginate solution (1.2%) at a concentration of 6×10⁶ cells/ml. The alginate solution will be slowly passed through a 22-gauge needle into a 125 mM CaCl₂ solution and allowed to precipitate for 10 min in CaCl₂ solution. The beads will be washed 2-3 times in 0.15 M NaCl and one wash in Ham's F-12 medium containing 10% FBS (Life Technologies, Inc.) and ascorbic acid (25 mg/ml) and seeded in 96 well plates containing chondrocyte growth medium Cells will be assayed for anabolic and catabolic responses at various time points following initiation of 3D culture.

These studies are designed to determine the distinct effects of F-spondin and its cleavage fragments on chondrocyte metabolism, as well as to dissect the discrete signaling pathways by which it acts. The use of a 3D alginate culture system will allow us to study the effects of F-spondin on chondrocytes within the context of an intact extracellular matrix over extended culture periods. Gene expression studies and the use of Affymetrix microarray analyses may also highlight the multi-function property of F-spondin with respect to matrix synthesis or degradation and other cellular pathway activation in chondrocytes. By inhibiting PGE2, TGF-β and αvβ3 pathways, we will be able to characterize discrete and separate signaling properties of F-spondin and thereby separate its anabolic and catabolic actions in an effort to define its role in disease pathogenesis.

5A. Effects of Overexpression of Full Length F-Spondin cDNA on Human Chondrocyte Functions

To determine the effects of F-spondin overexpression on chondrocyte function, human chondrocytes will be transduced with a MSCV retroviral vector encoding either full length F-spondin cDNA, or GFP, seeded into alginate gels at and assayed for anabolic and catabolic responses following periods of extended in vitro culture. Cultures will be incubated in the presence or absence of recombinant IL-1β (range: 1-10 ng/ml) to determine whether F-spondin antagonizes or enhances its catabolic effects.

Such experiments are performed routinely in our laboratory and are the subject of multiple prior publications (Attur M G, Dave M, Cipolletta C, Kang P, Goldring M B, Patel I R, et al. Reversal of autocrine and paracrine effects of interleukin 1 (IL-1) in human arthritis by type II IL-1 decoy receptor. Potential for pharmacological intervention. J Biol Chem 2000; 275(50:40307-15; Amin A R, Attur M, Patel R N, Thakker G D, Marshall P J, Rediske J, et al. Superinduction of cyclooxygenase-2 activity in human osteoarthritis-affected cartilage. Influence of nitric oxide. J Clin Invest 1997; 99(6):1231-7; Amin A R, Di Cesare P E, Vyas P, Attur M, Tzeng E, Billiar T R, et al. The expression and regulation of nitric oxide synthase in human osteoarthritis-affected chondrocytes: evidence for up-regulated neuronal nitric oxide synthase. J Exp Med 1995; 182(6):2097-102; Patel I R, Attur M G, Patel R N, Stuchin S A, Abagyan R A, Abramson S B, et al. TNF-alpha convertase enzyme from human arthritis-affected cartilage: isolation of cDNA by differential display, expression of the active enzyme, and regulation of TNF-alpha. J Immunol 1998; 160(9):4570-9). Cells will be cultured for 1, 2 and 4 weeks. This will allow us to compare chondrocyte responses in the presence of increasing accumulation of extracellular matrix. For each time point and group, we will perform immunohistochemistry on selected cultures using an antibody to F-spondin to monitor its deposition within the matrix, and its distribution among pericellular and interterratorial zones.

For initial experiments, chondrocytes isolated from OA knee joints will be used, building from our previous observations using monolayer cultures of these cells (Attur M G, Dave M, Cipolletta C, Kang P, Goldring M B, Patel I R, et al. Reversal of autocrine and paracrine effects of interleukin 1 (IL-1) in human arthritis by type II IL-1 decoy receptor. Potential for pharmacological intervention. J Biol Chem 2000; 275(51):40307-15). However, because OA chondrocytes endogenously express F-spondin, at least initially, there is a potential for a masking effect between control and F-spondin-transduced cultures. To address this, experiments will also be performed in both “normal” chondrocytes isolated from non-arthritic cartilage (obtained from NDRI, Philadelphia) and immortalized C28I2 chondrocytes, where endogenous F-spondin expression is low and absent, respectively (unpublished data). Using this experimental culture system, we expect similar metabolic responses among chondrocyte sources, however if variation between sources is observed, to F-spondin either with or without IL-1, they will be noted and investigated further. For each group and time point, alginate cultures and conditioned cell culture media supernatants will be analyzed by quantitative PCR, ELISA and biochemical assays for molecular markers associated with cartilage anabolism and catabolism. Table 1 below lists the molecules that will be analyzed for each group. Based on our preliminary findings with F-spondin and IL-1 treatment of monolayer chondrocytes, we predict that at least some of these molecules will be modulated in alginate cultures.

TABLE 1
Genes/gene products to be analyzed in chondrocyte metabolism studies

| Cytokines/Growth | | | | | |
|---|---|---|---|---|---|
| Factors | | Anabolic/Synthetic | | Catabolic/Inflammatory | |
| TNFα** | IL-1β** | SOX9 | AGC1* | PGE2* | MMP-13* |
| TGF-β* | BMP-2* | Runx2 | CBFA1 | Nitric oxide | MMP-1** |
| IL-8* | | COLXA1 | PG | COX-2 | Collagen, |
| IL-6* | | COL2A1* | synthesis | iNOS | aggrecan |
| | | | | NURR1 | fragments |

Asterisks denote F-spondin-mediated upregulation* or downregulation** in preliminary experiments with chondrocytes in monolayer culture.

We will also study the effect of F-spondin on global gene expression in these alginate cultures by microarray (Affymetrix). Based on our preliminary findings with IL-1 treatment of monolayer chondrocytes (Attur M D, M. Akamatsu, M. Tsunoyama, K. Yokota, H. Miki, J. Katoh, M. and Amin, A. Analysis of IL-1 specific global transcriptome profile in human chondrocyte and cartilage. In: 49th Annual Meeting of the Orthopeadic Research Society; 2003), we predict that a large number of genes from various functional groups (e.g., cytokines, chemokines, ECM proteins, etc) will be modulated by F-spondin. We will use GeneTraffic/SAM bioinformatics analyses to determine which gene clusters or metabolic pathways, predicted and unanticipated, are activated by F-spondin overexpression. Gene clusters related to catabolic, anabolic and developmental pathways are of particular interest.

5B. Effects of Blocking (siRNAs) F-Spondin in Human Chondrocyte Alginate Cultures

The purpose of this study is to determine whether blocking endogenous F-spondin activity in human chondrocytes affects the same metabolic pathways noted above. F-spondin activity will be blocked by knocking down its gene expression using siRNA oligos that target the 5′ end of the F-spondin gene. This approach is designed to block endogenously expressed F-spondin, thus normal and OA chondrocytes will be used for experiments. Chondrocytes will be modified with MSCV retroviral vectors encoding siRNA probes. Knockdown of gene expression will be confirmed by Western blot and RT-PCR. Following the timeline described above, siRNA-expressing alginate cultures will be analyzed for the various metabolic responses outlined in Table 1. Coupled with the results obtained from the other studies noted above, this study should provide verification of the metabolic and gene effects of F-spondin in chondrocytes. Distinct metabolic pathways activated only in the presence of relatively low F-spondin levels (such as in normal cartilage) and those activated when it is overexpressed (such as in OA) may also be identified.

### 5C. Effects of the Expression of Specific F-Spondin Domains on Human Chondrocyte Functions

The results from the experiments outlined above should identify specific anabolic and catabolic effects of F-spondin in cultured chondrocytes. We will then investigate whether particular F-spondin effects can be attributed to discrete regions of the F-spondin molecule and correspond to specific functional domains (FIG. 9a). For these studies, MSCV retroviral vectors encoding deletion constructs of F-spondin cDNA will be utilized based upon the predicted cleavage products of plasmin and other proteases (FIG. 13). Human chondrocytes will be transduced with the various constructs and cultured in alginate hydrogels as before. F-spondin-mediated metabolic effects, identified from experiments outlined above, will then be compared among transduced chondrocyte cultures to identify which of the truncated F-spondin fragments are biologically active. Since the expressed fragments are designed to reflect proteolytic degradation products, this study provides a biologically relevant context with which to examine the functional domains of F-spondin.

### 5D. To Determine the Effects of Exogenously Added F-Spondin and its Fragments on Chondrocyte Functions

We will determine if the F-spondin-mediated metabolic effects in alginate cultures are recapitulated by exogenous protein addition rather than via retroviral-mediated gene transfer. Purified F-spondin (R&D systems) will be added to alginate cultures to yield final concentrations ranging from 1-1000 ng/ml. The F-spondin-mediated metabolic effects from the studies noted above will then be measured for each dose. To verify the effects of functionally active F-spondin fragments from above, concentrated supernatants from retrovirus-transduced monolayer C28I2 chondrocytes expressing truncated F-spondin cDNAs will be added back to untransduced chondrocyte alginate cultures, as we have reported in Preliminary Studies (FIG. 11). These experiments are designed to confirm F-spondin-mediated activity in non-transduced cultures and also examine the relationship between F-spondin dose and its metabolic effects.

### 5E. Distinctive Effects of F-Spondin are Mediated by Engagement of Discrete and Separate Signaling Pathways

As illustrated in FIG. 13, we hypothesize that the effects of F-spondin on chondrocyte functions observed in monolayer cultures are due to both: 1) distinct properties of the intact molecule and its proteolytic fragments and 2) activation of discrete and separate signaling pathways that result from the biological actions of F-spondin in the extracellular matrix. Our preliminary findings suggest that F-spondin acts upon chondrocytes via at least three signal transduction pathways: 1) prostaglandin E2/cAMP/NURR1 (FIG. 11); 2) TGF-β1 (FIG. 12) and 3) outside-in signaling via ligation of the αvβ3 integrin (FIG. 11). In these studies, we will determine which and how the F-spondin metabolic responses are attributed to these three separate activation pathways.

### 1) Prostaglandin-Mediated Effects

To investigate PGE2/cAMP/NURR1 pathway activation, cultures will be incubated with three pathway inhibitors: 1) the COX-2 inhibitor, celecoxib or the dual COX-1/COX-2 inhibitor ibuprofen, at concentrations sufficient to inhibit PGE2 production by >75%; 2) 1-10 μM of A23858, an EP4 receptor antagonist (Sigma); or 3) 1-10 μM H-89 of the PKA inhibitor (Calbiochem).

### 2) TGF-β-Mediated Effects

TGF-β signaling will be inhibited by treatment with varying concentrations of anti-TGF β antibody, which neutralizes multiple isoforms of TGF-β (R&D systems). Isotype control antibodies will serve as controls in these experiments.

3) αvβ3 Integrin-Mediated Effects

αvβ3 signaling will be blocked by treatment with 5-10 μg of blocking antibody LM609 (Chemicon International). Inhibitors will be added for 24-72 h in alginate cultures after 1, 2 or 4 weeks. The mode of F-spondin addition i.e. transgenic vs protein, full length vs truncated fragment, and its subsequent metabolic responses will be determined from the previous studies.

### Example 6

### The Role of F-Spondin on Chondrocyte Differentiation

### F-Spondin Regulates Chondrocyte Hypertrophy and Maturation

We have demonstrated for the first time that there is preferential distribution of F-spondin in the hypertrophic and calcified regions of the chick embryo growth plate (FIG. 5), suggesting that the expression of the protein may be linked to the state of maturation of the cells. However, the role of F-spondin in chondrocyte differentiation has not been fully investigated. We use in vitro cultures of chick embryonic cells and adult mesenchymal stem cells (MSCs) to investigate the link between chondrocyte hypertrophy and F-spondin. Our results provided below demonstrate that F-spondin is expressed in embryonic growth plate cartilage and can enhance the expression of chondrocyte maturation markers.

Chick embryo cultures are a well-established in vitro system that permits the study of the temporal events associated with chondrocyte maturation (Iwamoto M, Shapiro I M, Yagami K, Boskey A L, Leboy P S, Adams S L, et al. Retinoic acid induces rapid mineralization and expression of mineralization-related genes in chondrocytes. Exp Cell Res 1993; 207(2):413-20; Iwamoto M, Yagami K, Shapiro I M, Leboy P S, Adams S L, Pacifici M. Retinoic acid is a major regulator of chondrocyte maturation and matrix mineralization. Microsc Res Tech 1994; 28(6):483-91). MSCs, isolated from postnatal human tissue, also undergo chondrogenesis in defined culture conditions and appear to mimic the differentiation events observed in embryonic chick cells (Johnstone B, Hering T M, Caplan A I, Goldberg V M, Yoo J U. In vitro chondrogenesis of bone marrow-derived mesenchymal progenitor cells. Exp Cell Res 1998; 238(1):265-72).

### Materials and Methods:

### Chick Embryonic Cell Culture and Transfection

Chondrocytes were isolated from the cephalic and caudal portion of day 14 chick embryo sterna as described by (Teixeira C C, Shapiro I M, Hatori M, Rajpurohit R, Koch C. Retinoic acid modulation of glutathione and cysteine metabolism in chondrocytes. Biochem J 1996; 314 (Pt 1):21-6). After 5 days of primary culture, chondrocytes were separated from the attached fibroblasts, harvested by centrifugation, counted, and plated in tissue culture dishes in complete medium supplemented with hyaluronidase (4-units/ml) to promote attachment. After becoming confluent, and to induce maturation, cells were treated daily with all-trans retinoic acid (35-100 nM) (Sigma) in the presence of ascorbate-2-phosphate (100 μM) for 1 week. Control cultures were treated with an equal volume of vehicle. Cultures were provided with β-glycerophosphate (5 mM) to serve as phosphate donor. For transfections, chondrocytes were transiently transfected with a plasmid encoding full length F-spondin cDNA under control of a CMV promoter, 1 day after plating in monolayer culture, using the FuGene 6 Transfection Reagent (Roche, Nutley, N.J.) according to the manufacturer's specifications. Parallel transfections with a CMV-GFP plasmid was used as a null control and to estimate transfection efficiency.

### Histology and Immunohistochemistry of Aggregate Cultures

Aggregate cultures were fixed, paraffin embedded, sectioned and stained for evidence of cartilage proteoglycan matrix synthesis by toluidine blue staining. For immunohistochemistry, sections were incubated with rabbit polyclonal antibodies to collagen type I, II X, COMP and F-spondin (purchased from Rockland or R&D systems). Positive staining was visualized by fluorescence microscopy following incubation of fluorescein conjugated anti-rabbit IgG secondary antibodies.

### Gene Expression Analyses by RT-PCR

RT-PCR analyses was used to characterize temporal expression of chondrogenic activity at the molecular level. Type II collagen α1 chain, type I collagen α2, type X collagen α1, aggrecan, COMP and F-spondin mRNA were each amplified suing specific primer sets (these human primers are commercially available from Applied Biosystems for real time PCR analysis). Analysis of PCR products from each of the chondrogenic cultures provides an indication of the timing and duration of expression of F-spondin and other chondrogenic marker genes.

### MSC Culture and Differentiation Assays

For these studies we will use primary human MSCs obtained from the Tulane Center for Gene Therapy distribution program. Prior to experiments, primary MSCs will be maintained as low density monolayer cultures in alpha MEM containing 10% FBS (screened lot). For chondrogenic assays, cells will be trypsinized and centrifuged to form high density aggregates at a concentration of 6×10⁵ cells/ml. The aggregates will be cultured in chondrogenic induction medium, consisting of serum-free DMEM containing 1% ITS+premix, 10⁻⁷M dexamethasone, and 100 μM ascorbate. Chondrogenesis will be induced by addition of either 50 ng/ml TGF-β1, 50 ng/ml TGF-β 3, 50 ng/ml BMP-2 or 100 ng/ml IGF-1 (all purchased from R&D systems). These growth factors have been reported to induce chondrogenesis of adult MSCs to varying degrees. We will compare their effects on chondrogenesis and the relationship to F-spondin expression. Histology, immunohistochemistry, and gene expression analyses will be performed as described above.

### Osteogenic and Adipogenic Differentiation Assays

For osteoinduction, MSCs will be cultured as monolayers in the presence of serum supplemented with DMEM containing 20 mM b-glycerolphosphate, 10⁻⁷ M dexamethasone and 100 μM ascorbate, according to the method of Haynesworth/Caplan (Haynesworth S E, Goshima J, Goldberg V M, Caplan A I. Characterization of cells with osteogenic potential from human marrow. Bone 1992; 13(1):81-8). After 2 weeks, cultures will be analyzed for evidence of osteogenesis by alzarin red staining of mineral deposits and alkaline phosphatase. Gene expression of osteogenic markers, cbfa1, osteopontin and osteocalcin will also be confirmed by RT-PCR. To induce adipocyte differentiation, MSC monolayers will be cultured in serum containing DMEM supplemented with 10 ng/ml Insulin, 10⁻⁶ M dexamethasone, and 0.5 μM IBMX for 2 weeks. Adipogenesis will be assessed by Oil Red O staining for visualization of lipid droplets, and RT-PCR for adipogenic markers PPARγ and adipsin.

6A. The Role of F-Spondin on Chondrocyte Maturation of Chick Embryo Chondroprogenitor Cells. F-Spondin Expression is Localized to Maturing Chondrocytes in the Tibial Growth Plate.

Tibial growth plate sections from 18 d old chick embryos were stained with either F-spondin antibody or control IgG. Positive F-spondin immunostaining (brown) was detected in only the hypertrophic and calcified regions. While abundant staining for F-spondin was seen within the hypertrophic and ossifying regions, no staining was observed in the immature chondrocytes of the resting and proliferating zones. This expression pattern implicates a role for F-spondin in late-stage terminal differentiation. Cartilage tissue corresponding to proliferative (P), hypertrophic (H) and calcified (C) regions of a tibial growth plate were harvested by microdissection and assayed for gene expression by qPCR. Relative expression of F-spondin and other cartilage maturation markers are displayed in Table 2.

TABLE 2
mRNA Levels in Growth Plate Chondrocytes
(% Change from PROLIFERATIVE)

| GENE | PROLIFERATIVE | HYPERTROHIC | CALCIFIED |
|---|---|---|---|
| Type II | 100 | 16.5 | 1.6 |
| Collagen | | | |
| Type X | 100 | 417 | 232 |
| Collagen | | | |
| MMP13 | 100 | 708 | 939 |
| VEGF | 100 | 252 | 194 |
| f-spondin | 100 | 109 | 201 |
| Type I | 100 | 323 | 1908 |
| Collagen | | | |

### Growth Plate Maturation can be Mimicked in Cell Culture Following Retinoic Acid (RA) Treatment of Chick Cephalic Chondrocytes

Chondroprogenitor cells isolated from the cephalic portion of embryonic chick sterna and treated with retinoic acid (RA) mimic the changes observed in vivo during growth plate chondrocyte maturation and hypertrophy (Silvestrini G, Mocetti P, Ballanti P, Di Grezia R, Bonucci E. In vivo incidence of apoptosis evaluated with the TdT FragEL DNA fragmentation detection kit in cartilage and bone cells of the rat tibia. Tissue Cell 1998; 30(6):627-33). These cells promptly express type X collagen, increase their alkaline phosphatase activity and exhibit rapid mineral deposition (Iwamoto M, Shapiro I M, Yagami K, Boskey A L, Leboy P S, Adams S L, et al. Retinoic acid induces rapid mineralization and expression of mineralization-related genes in chondrocytes. Exp Cell Res 1993; 207(2):413-20; Iwamoto M, Yagami K, Shapiro I M, Leboy P S, Adams S L, Pacifici M. Retinoic acid is a major regulator of chondrocyte maturation and matrix mineralization. Microsc Res Tech 1994; 28(6):483-91) which are markers for maturation. Alkaline phosphatase (AP) expression (red color) is a common marker of chondrocyte maturation. In contrast, cells isolated from the caudal portion of the sterna do not undergo maturation in response to RA. They represent a more immature state of chondrocyte differentiation and, as such, these cells can be used as negative controls for studies of maturation related events in vitro.

To investigate the role of F-spondin in chondrocyte maturation, we used an in vitro model in which chick sternal chondrocytes mimic growth plate chondrocytes and undergo terminal differentiation in response to RA treatment. RA stimulation for 5 days (10-100 nm) resulted in a 2- to 4-fold increase in F-spondin gene expression when compared to non-stimulated controls. Chick chondrocytes were stimulated with increasing doses of RA (10-100 nm) and harvested for gene expression analysis by qPCR after 5 days. The relative gene expression of type X collagen (ColX), alkaline phosphatae (AP), MMP-13, and F-spondin were assessed with increasing maturation on RA stimulation (FIG. 14). F-spondin expression increases with maturation, replicating its expression profile in the tibial growth plate. These results demonstrate that F-spondin is expressed in the developing growth plate where it acts to enhance terminal differentiation of chondrocytes via upregulation of hypertrophic markers.

Identification of Hypertrophic Markers that Respond to Increased Expression of F-Spondin.

F-spondin overexpression was found to induce expression of chondrocyte maturation genes, AP and MMP-13, following RA treatment. Chick chondrocytes were transfected with either F-spondin or vector control (pcDNA3) and stimulated with RA to induce maturation. Expression of F-spondin in cultured chondrocytes was increased by transfection with a plasmid encoding full length F-spondin cDNA. Briefly, cephalic or caudal sternal chondrocytes were transfected one day after secondary plating with the F-spondin plasmid or null construct as control and cultured in the presence or absence of the maturation agent retinoic acid (35-100 nM (RA). mRNA was collected for semi-quantitative real time RT-PCR analysis of chondrocyte markers (type II, and type X collagen, alkaline phosphatase, Runx-2 and MMP13). Overexpression of F-spondin cDNA by plasmid transfection, increased expression of chondrocyte terminal differentiation markers, MMP-13 (5-fold) and alkaline phosphatase (AP) (14-fold) in cultures stimulated with RA (p<0.05) (FIG. 15). F-spondin overexpression also enhances AP enzyme activity and mineralization of RA-treated chick chondrocytes. Chondrocytes were transfected as previously and assayed for AP activity by ELISA, or calcium deposition by Von Kossa staining with alizarin red (39). Both AP activity and amount of calcium deposition were significantly increased with F-spondin overexpression (data not shown). However, these effects were not observed in F-spondin transfected cultures without RA stimulation, suggesting that F-spondin acts as an enhancer rather than an inducer of terminal differentiation.

### Effect of F-Spondin Blocking Antibodies on Chondrocytes Maturation and Mineralization Markers

F-spondin function was inhibited using anti F-spondin antibody and the effect on different maturation markers investigated. Inhibition of F-spondin was found to decrease AP activity in RA stimulated cultures. Chick chondrocytes were treated with RA with and without F-spondin antibodies (R&D Systems Cat No. 3135-SP/CF) Antibodies with specificities to the spondin domain and TSR domain inhibited AP activity compared to control (no Ab) as shown in FIG. 16. The TSR domain antibody had a greater inhibitory effect. Consistent with the above findings, blocking F-spondin activity via addition of polyclonal F-spondin antibodies resulted in an inhibition of RA-induced chondrocyte maturation, evidenced by decreased AP activity. The level of inhibition was dependent on the antibody specificity to F-spondin protein domains; blocking the c-terminal, thrombospondin-like TSR domain caused a 50% (p<0.01) inhibition of AP activity, while targeting the n-terminal, spondin domain reduced this effect to 2-10%. This finding suggests that F-spondin induction of AP during chondrocyte maturation is mediated via its TSR domain.

We next determined that the pro-maturation effect of F-spondin is not inhibited following neutralization of TGF-β activity by coculture with Latency Associated Peptide (LAP) (R&D Systems, Cat No 246-LP/CF). TGF inhibition by LAP accelerates maturation and does not diminish the effect of F-spondin. AP activity was determined in chick chondrocyte cultures by ELISA following RA stimulation for 3 days. Cultures were stimulated with RA alone or in combination with either F-Spondin (1 ug/ml) or LAP (10-100 ng/ml) both alone and in combination (FIG. 17). The addition of LAP did not significantly alter the AP activity increase seen with F-spondin alone.

Blocking αvβ3 Integrin Inhibits the Promaturation Effect of F-Spondin.

Chondocyte cultures were transfected as previously and stimulated with RA for 3 days in the presence of IgG control, or αvβ3 blocking antibodies (antibody LM609, Chemicon International, Cat NoMAB1976Z) prior to assay for AP activity. Inhibition of αvβ3 by the blocking antibody led to about 50% suppression of F-spondin-induced AP activity but had no effect on baseline AP activity (FIG. 18).

### 6B. The Role of F-Spondin on Chondrogenic Differentiation of Postnatal Mesenchymal Stem Cells (MSCs).

While not as well characterized as the chick model, postnatal MSCs also undergo chondrogenesis and appear to hypertrophy after 21 days in culture, evidenced by enhanced expression of type X collagen and alkaline phosphatase (Johnstone B, Hering T M, Caplan A I, Goldberg V M, Yoo J U. In vitro chondrogenesis of bone marrow-derived mesenchymal progenitor cells. Exp Cell Res 1998; 238(1):265-72; Barry F, Boynton R E, Liu B, Murphy J M. Chondrogenic differentiation of mesenchymal stem cells from bone marrow: differentiation-dependent gene expression of matrix components. Exp Cell Res 2001; 268(2):189-200; Palmer G D, Steinert A, Pascher A, Gouze E, Gouze J-N, Betz O, et al. Gene-Induced Chondrogenesis of Primary Mesenchymal Stem Cells in vitro. Molecular Therapy 2005; 12(2):219-228; Yoo J U, Barthel T S, Nishimura K, Solchaga L, Caplan A I, Goldberg V M, et al. The chondrogenic potential of human bone-marrow-derived mesenchymal progenitor cells. J Bone Joint Surg Am 1998; 80(12):1745-57). TGF-β and BMP superfamily growth factors are typically required to stimulate chondrogenesis in these cells, but their effects on chondrocyte maturation vary. To investigate the role of F-spondin on chondrogenesis of postnatal, human MSCs, studies are performed using aggregate cultures exposed to the chondroinductive growth factors, TGF-β1, -β3, BMP-2 and IGF-1. These studies are aimed to establish a more detailed expression profile of F-spondin following exposure to chondrogenic growth factors and examine its effects on the chondrogenic fate of MSCs when its expression is blocked or increased.

### Analysis of F-Spondin Expression During Chondrocyte Differentiation of Post Natal, Human MSCs.

Aggregate cultures of MSCs are incubated with growth factors commonly used in chondrogenesis studies: TGF-β1 and β 3, BMP-2 or IGF-1. Aggregates will be analyzed for marker gene expression of extracellular matrix molecules day 0, prior initiation of chondrogenesis, and from aggregates incubated with the various growth factors at days 4, 14 and 21. The onset of F-spondin expression is determined in aggregates exposed to the various growth factors and its expression compared with other chondrocyte maturation markers including COMP, alkaline phosphatase and collagens I, II and X. Expression of markers is measured by RT-PCR and immunohistochemistry of aggregate sections.

### Effect of F-Spondin Blocking Antibody on Chondrocyte Differentiation.

The effects of blocking F-spondin during chondrogenesis of MSCs is determined in an additional set of experiments. Aggregate cultures are incubated with F-spondin antibodies at various time points prior to the onset of its expression (as determined above). The resulting effects on chondrogenesis, proteoglycan matrix staining and expression of chondrogenic markers as described above, are determined after 21 days.

### Determination of MSC Fate Following Overexpression of F-Spondin.

The retroviral construct encoding full length F-spondin cDNA and an IRES GFP (described above) is used to genetically modify primary MSCs. Transduced cells are sorted by FACS and expanded in monolayer cultures. Following expansion, MSCs are induced to either osteogenic, adipogenic or chondrogenic differentiation. Differentiation is assessed by histologic staining or RT-PCR of lineage-specific markers. For each group, the extent of differentiation is compared to control groups modified with a retroviral construct encoding GFP only.

### 6C. Identification and Characterization of the 5′ Promoter Regulatory Region of the Human F-Spondin Gene.

Because of its relatively unique expression profile in developing cartilage and upregulation in OA, F-spondin provides a novel target with which to study molecular regulation of gene expression in chondrocytes. To understand the regulation of F-spondin expression in chondrocytes, we sought to clone, identify and characterize the promoter regulatory region governing the expression of F-spondin. Since the human genome is completely sequenced, we utilized this information to retrieve the 5′ upstream sequence of F-spondin using S.O.U.R.C.E genome database from Stanford. The upstream sequence was further analyzed using TFSEARCH program to look for possible transcription factor binding sites. From the initial screen it is observed that F-spondin may be regulated by multiple transcription factors such as CREB (cAMP responsible element binding proteins), CCAAT/enhancer binding proteins (C/EBPB), NFkB, sex-determining region Y gene product (SRY) etc. Further characterizing the promoter region of the F-spondin gene will enable us to identify cis- and trans-acting factors that regulate chondrocyte differentiation and potentially OA progression.

### Cloning of 5′ Upstream Regulatory Region by RT-PCR.

BD Genome Walker kit is used for cloning the promoter and regulatory elements. The gene specific primer is: 5′ CTTCGTCGGGACCACTTCGGGCAGGAGTCGCGTGGCGAAGGC 3′ (SEQ ID NO: 33) and API primer supplied in the kit is used to amplify the 5′ upstream region and then cloned in TA cloning vector. The nucleotide sequence is verified by bi-directional sequence walking with gene specific primers. Promoter and transcription factor binding site prediction are performed using McPromoter and TSSG/TSSW prediction programs. The promoter fragment is sub-cloned into promoterless luciferase reporter plasmid (pGL3) at appropriate restriction sites. The full length promoter construct is used for various deletion constructs either by using appropriate restriction site or by amplifying the fragment by RT-PCR with specific internal primers with appropriate restriction sites and cloning into a promoter vector.

### Transient Transfection and Luciferase Assay.

To verify promoter activity, human OA chondrocytes—which express endogenous F-spondin in monolayer cultures following isolation—are transfected with the F-spondin promoter luciferase plasmid using either FuGene 6 or AMAXA protocols. Control luciferase reporter plasmids, pGL3-basic, pGL3-control and pGL3-enhancer (Promega) are transfected in parallel to provide a comparison for promoter strength. Reporter gene expression is compared to human C28I2 chondrocyte cells, which have no endogenous F-spondin expression. To determine if activity of the F-spondin promoter is regulated during chondrogenesis, embryonic chick cells are isolated and transfected (as described above) with the F-spondin promoter luciferase construct and induced to undergo chondrogenesis by addition of maturation agents. At 4, 24 and 48 h following transfection (using the same timeline described above) cells are harvested and assayed for promoter activity. Further deletional analyses are then performed to identify putative cis-acting regulatory regions. In all transfection studies, n-gal expression vector pCMV-gal is co-transfected as an internal control for transfection efficiency.

### Example 7

### Identification and Characterization of the Interacting Proteins of F-Spondin

F-spondin is a multi-domain glycoprotein, which has the potential to interact with other ECM proteins or cell surface receptors. Important biological functions of F-spondin on chondrocyte metabolism may be mediated by protein-protein interactions between the functional domains of F-spondin (reelin, spondin, TSR) and ECM proteins, latent TGF-β1, proteases (including plasmin and yet unidentified proteases) and cell surface receptors.

### 7A. The Utilization of Y2H and Co-IP/Proteomic Strategies to Identify F-Spondin-Binding Proteins

Using Y2H strategies, we have identified ADAMTS-7 and 12 as binding partners for COMP, an ECM protein, which like F-spondin, is a member of a family of proteins that belong to the subgroup of TSR (thrombospondin) type I class molecules (Liu C J, Kong W, Ilalov K, Yu S, Xu K, Prazak L, et al. ADAMTS-7: a metalloproteinase that directly binds to and degrades cartilage oligomeric matrix protein. Faseb J 2006; 20(7):988-90; Liu C, Kong W, Xu K, Luan Y, Ilalov K, Sehgal B, et al. ADAMTS-12 associates with and degrades cartilage oligomeric matrix protein. J Biol Chem 2006). Hoe et al., using co-immunoprecipitation techniques, have demonstrated that F-spondin interacts with an apoE receptor through its thrombospondin domain and the ligand binding domain of ApoEr2 (Hoe H S, Wessner D, Beffert U, Becker A G, Matsuoka Y, Rebeck G W. F-spondin interaction with the apolipoprotein E receptor ApoEr2 affects processing of amyloid precursor protein. Mol Cell Biol 2005; 25(21):9259-68). Selected biological functions of F-spondin on chondrocyte metabolism may be mediated by protein-protein interactions between the functional domains of F-spondin (reelin, spondin TSR) and ECM proteins, proteases and cell surface receptors.

Strategies previously employed to identify COMP-binding proteins are utilized to isolate the binding partners of F-spondin (Liu C, Kong W, Xu K, Luan Y, Ilalov K, Sehgal B, et al. ADAMTS-12 associates with and degrades cartilage oligomeric matrix protein. J Biol Chem 2006; Liu C, Dib-Hajj S D, Waxman S G. Fibroblast growth factor homologous factor 1B binds to the C terminus of the tetrodotoxin-resistant sodium channel rNav1.9a (NaN). J Biol Chem 2001; 276(22):18925-33). Focus is made particularly on 1) proteases, including but not limited to plasmin, that may cleave F-spondin, 2) cell surface receptors, including but not limited to αvβ3 and 3) previously unidentified binding proteins of potential biological interest. This is achieved through a series of molecular and proteomic approaches which are described below and also based on methods known in the art.

### Materials and Methods

cDNA Library Construction

A two-hybrid cDNA library is prepared from RNA isolated from 20 OA or non-arthritic cartilage specimens. RNA will be pooled to get sufficient polyA mRNA for cDNA library preparation. The Superscript Plasmid system cDNA synthesis and the pPC86 plasmid cloning kit (Invitrogen) are used. These libraries have been used previously (Liu C, Kong W, Xu K, Luan Y, Ilalov K, Sehgal B, et al. ADAMTS-12 associates with and degrades cartilage oligomeric matrix protein. J Biol Chem 2006; Liu C, Dib-Hajj S D, Waxman S G. Fibroblast growth factor homologous factor 1B binds to the C terminus of the tetrodotoxin-resistant sodium channel rNav1.9a (NaN). J Biol Chem 2001; 276(22):18925-33). Additionally, we have previously constructed normal and OA cartilage expression cDNA library using Strategene pBK-CMV vectors to clone various matrix metalloproteinases (MMPs) and the TNFα converting enzyme (TACE) (Attur M G, Dave M, Cipolletta C, Kang P, Goldring M B, Patel I R, et al. Reversal of autocrine and paracrine effects of interleukin 1 (IL-1) in human arthritis by type II IL-1 decoy receptor. Potential for pharmacological intervention. J Biol Chem 2000; 275(51):40307-15).

### Yeast Two-Hybrid Library Screen

Strategies that have led to identification of protease ADAMTS-7 and 4 by Y2H, which associates and cleaves COMP as shown in preliminary studies (data not shown) are utilized. A cDNA segment encoding the reelin and spondin domain (aa 43-173), spondin domain (174-415), (TSR (1-6) domain (aa 446-807) and full length (aa 24-807) are amplified by PCR and cloned in-frame into the SalI/NotI sites of pDBleu (Life Technologies) to serve as bait to screen the yeast expression libraries. Bait plasmids are introduced into an MaV203 yeast strain, selected and them transformed with a pPC86 cDNA library as described (30). The recombinant individual clones are sequenced and a blast search performed using the NCBI database to characterize the clones.

In Vivo and In Vitro Interaction of Factor ‘X’ with F-Spondin and its Expression Pattern

Following further identification of factors (eg factor X (s)) including by the procedure described above, F-spondin binding to this factor is verified employing in vitro pull down assays and in vivo Co-IP assays. Factor X may be cloned in-frame into an expression vector with a His-tag. An Ig fused C-terminal of F-spondin (TSR-1-6, FIG. 9) immobilized to protein A beads (Pharmacia) is used to pull down His-tagged X in vitro; cell lysates transfected with His-tagged F-spondin and Flag- or HA-tag factor X are immunoprecipitated with anti-F-spondin antiserum and immunoblot analysis performed with anti-HA or Flag antibody to detect factor X. expression plasmids encoding full-length F-spondin and any factor X (s) are cotransfected into HEK293 cells and dual indirect immunofluorescence is used to determine F-spondin and factor X co-localization.

### Expression and Purification of Ig F Spondin or Ig Mindin for Pull Down Assay to Identify Interacting Proteins by MS.

Full-length Ig F-spondin and Ig Mindin cDNAs are used for pull down assays to identify interacting proteins by MS. Mindin (which has only one TSR domain) is another member of non-thrombospondin family and will be used as a negative control. Expression vectors expressing full length F-spondin and Mindin are expressed in C28I2 cells. To purify the secreted Ig fusion protein, supernatants are harvested after 24 h to 48 h transfection, centrifuged, filtered, and applied to an anti-human IgG column (ICN Biomedicals, Eschwege, Germany). After elution and dialysis, protein concentrations are determined photometrically at 280 nm.

### Pull Down Assay Using Immobilized Ig-Fusion Proteins.

Total protein is extracted from OA chondrocytes, solubilized and incubated overnight at 4° C. with Ig-F-spondin or Ig-Mindin protein immobilized on Protein A sepharose. The are then resuspended in sample buffer, boiled and analyzed by SDS-PAGE on 10% Criterion gels (Bio-Rad, Hercules, and CA) and further analyzed by liquid chromatography MS of tryptic fragments

Identification of Proteins from SDS-PAGE Gels by MS.

After electrophoresis the proteins are visualized by Coomassie blue staining or a mass pectrometry-compatible silver staining kit (Invitrogen Inc.). Gel bands are excised from each lane destained, and the proteins digested in-gel with trypsin. The resulting peptides are extracted and analyzed using nanoflow LC/ESI-MS-MS. The Q-TOF Micro (Micromass, Manchester, United Kingdom) data acquisition involves MS survey scans and automatic data-dependent MS/MS acquisitions. The raw MS data are subsequently processed using manufacturer-supplied Protein-Lynx software. Control experiments demonstrate the sensitivity of this system is better than 100 fmol of each protein in the gel, and 10 fmol of each peptide injected onto the HPLC columns.

### Bioinformatics

The raw MS data is processed using Micromass ProteinLynx 3.5 software. Each query is used to search the most recent NCBI nr and Swissprot databases using Mascot version 2.1 (47) (Matrix Science, London, United Kingdom).

### Example 8

### Investigation of the Capacity of F-Spondin to Activate Latent TGF-β1

Thrombospondin (TSP-1) has been shown to bind directly to latent TGF-β via a WSxW motif found in each of three TSR repeats. This interaction facilitates the ability of a second motif, KRFK, found between 1^{st} and 2^{nd} TSR repeats to bind to the LAP portion of the TGF-β molecule and mediate the release the active TGF-β dimer. Peptides containing WSxW docking sequences have been used to competitively block activation of TGF-β by TSP-1 in both a chemically defined system and in various cell culture systems (Murphy-Ullrich J E, Poczatek M. Activation of latent TGF-beta by thrombospondin-1: mechanisms and physiology. Cytokine Growth Factor Rev 2000; 11(1-2):59-69). Like TSP-1, F-spondin also has conserved WSxW and KRFK motifs. We hypothesize that F-spondin binds to and activates latent TGF-β and provide data in cartilage explants consistent with this (FIG. 12). C2812 cell lines expressing F-spondin and F-spondin deletion mutants are generated and their ability to induce and activate TGF-β assessed by performing sequential experiments, described below.

### Materials and Methods

### Generation of F-Spondin and Deletion Mutants

To investigate the protein motifs involved in activation of latent TGF-β₁, C2812 cell lines are established expressing F-spondin and F-spondin deletion mutants. Site directed mutagenesis using the QuickChange Site—directed mutagenesis kit (Strategene, Germany) is performed to modify the KRFK motif to KRIR to create mutant F-spondin. Based on the observation by Schultz-Cherry S et al (1995) that TSP-1 can activate latent TGF-β₁ but TSP-2 cannot due to lack of KRFK motif and that, additionally, the KRFK but the not KRIR peptide, completely blocked complex formation between LAP and TSP-1, we presume that the mutant F-spondin, mutated to the KRIR motif, will not activate latent TGF-β₁ (Schultz-Cherry S, Chen H, Mosher D F, Misenheimer T M, Krutzsch H C, Roberts D D, et al. Regulation of transforming growth factor-beta activation by discrete sequences of thrombospondin 1. J Biol Chem 1995; 270(13):7304-10). For each cell line, total protein is extracted and immunoblot analysis performed to confirm expression of the various truncated constructs. For initial experiments a set of constructs is generated with point mutations. Following confirmation of protein expression, the % activation of latent TGF-β₁ is measured within the different groups by ELISA.

### Construction and Characterization of Stable C28I2 Cell Lines.

The human chondrocyte cell line C28I2 is transfected with various constructs of F-spondin in a pCMV-Ig vector with nucleofector reagent (Amaxa) and selected for resistance to puromycin. A number of puromycin resistant clones is selected and expression of various fragments of F-spondin is confirmed using two different antibodies recognizing different regions of F-spondin, R1 (which recognizes the TSR 3-6 domain) and R4 (recognizes N-terminal spondin domain) by western blot analysis. Once clones are characterized the supernatants from these stable cell lines is collected and concentrated using microcon 3 kDa cut off (Ambion) for TGF-β measurement studies. As negative controls, human albumin and a stable C28I2 cell line expressing another member of non-thrombospondin family mindin, which has only one TSR domain are used.

### 8A. Measurement of Total (Latent and Active) TGF-β in Supernatants of F-Spondin Transfected Cells

Total and active TGF-β (R&D systems) are measured in the supernatants of C28I2 stable cell lines expressing mutant and wild type F-spondin constructs. The measurement of increased levels of active TGF-β can be the result of increased latent TGF-β expression and secretion as well as increased latent TGF-β activation. To distinguish between these possibilities, measurement of the total/TGF-β (active+latent) is performed. The latent TGF-β is activated by acidification (as recommended by manufacturer, R&D Systems) of the media, thereby permitting measurement of total TGF-β present. This provides information on the induction of TGF-β expression by F-spondin in chondrocyte cells. The ELISA data establishes whether released TGF-β is biologically active. This is addressed this using a cell based reporter assay system, as described below.

### 8B. Determination of Biologically Active TGF-β in Culture Supernatants

Biologically active TGF-β is assayed using cultures of mink lung epithelial cells stably expressing a luciferase construct, p800neoLUC, under the control of TGF-β responsive elements (Abe M, Harpel J G, Metz C N, Nunes I, Loskutoff D J, Rifkin D B. An assay for transforming growth factor-beta using cells transfected with a plasminogen activator inhibitor-1 promoter-luciferase construct. Anal Biochem 1994; 216(2):276-84). Supernatants collected from various F-spondin expressing cell lines are added to the cultures and luciferase activity measured after 16-20 h. Specificity of the signal is determined by incubation of the samples with either LAP, which inhibits TGF-β activity, or specific anti-TGF-β neutralizing antibodies (R&D System). The results are presented as % of samples incubated in the absence of LAP or anti-TGF-β.

### 8C. Detection of TGF-β Signal Pathway Activation by Nuclear Localization of Smad2 and 3

TGF-β signaling is examined as a surrogate measure of active TGF-β levels by examining the nuclear localization of phosphorylated Smad proteins in primary human chondrocytes. Upon binding of active TGF-β to receptors, smad2/3 are phosphorylated and move from cytoplasm to nucleus. Thus, nuclear localization of p-smad2/3 can be used as a measure of TGF-β signaling. Human chondrocytes grown on glass coverslips are serum-starved for 24 h and incubated for 5-120 min with supernatants collected from various constructs of F-spondin expressing cells. After fixing and permeabilization, cells are incubated with polyclonal abs to Smad2/3 (Cell Signaling) for 1 h, followed by addition of a PE-conjugated goat anti-rabbit IgG (Vector Laboratories). Cultures are examined by fluorescent microscopy (Olympus1X71, IPLab program) Cells are counter stained with DAPI for nuclear staining. The images are superimposed.

### Example 9

### Investigation of the Expression and Function of F-Spondin in Cartilage In Vivo

Preliminary studies in both human and rat osteoarthritis demonstrate upregulation of F-spondin expression in cartilage (FIGS. 1a and 4). Given the effects that we have observed on both anabolic and catabolic chondrocyte functions, F-spondin is proposed to play a role in the development of disease. The role of F-spondin in bone and cartilage is assessed in vivo using a surgically induced mouse model of osteoarthritis (OA). Using the C57BL/6J strain, OA is induced by medial meniscectomy (Appleton et al., submitted; McErlain et al Osteo Cartilage 2007 Sep. 25 Epub). Previous microarray studies revealed a seven-fold upregulation of F-spondin mRNA in the operated joints compared to sham controls, consistent with our observations in human OA cartilage. Surgically induced rodent models therefore provide a relevant context with which to study F-spondin and its possible functions in OA. Accordingly, transgenic mice overexpressing F-spondin are generated to study its function in bone and cartilage development, as well as its role in OA following surgical-induction of skeletally mature animals.

### Materials and Methods

### Immunohistochemistry

Sample preparation and immunohistochemistry is performed as described (Wang et al, submitted; Appleton et al, submitted; Wu et al (2007) Arthritis Rheum November 56(11):3675-84; McErlain et al Osteo Cartilage 2007 Sep. 25 Epub). Freshly dissected bones are fixed with 10% buffered formalin and decalcified with 0.1 M EDTA. Specimens are embedded and sectioned at the Molecular Pathology Core Facility (London, ON). 5- to 8-mm sections are dewaxed and stained with hematoxylin/eosin or Safranin 0/fast green using standard protocols. For immunohistochemistry, sections are blocked with H₂O₂, treated to unmask antigens and incubated with above antibodies overnight at 4°. After secondary Ab addition, the sections are incubated for 2-10 minutes with DAB substrate solution, washed and mounted. For BrdU labeling, mice are injected intraperitoneally with BrdU (10 mg/ml; Roche) at a dose of 0.01 ml/g body weight one hour before sacrifice. Limbs are processed for paraffin sections as described above. BrdU incorporation is detected using an anti-BrdU antibody (Zymed Laboratories). TUNEL assay is performed using the DeadEnd™ Fluorometric TUNEL System (Promega) according to the manufacturer's instructions.

### In Situ Hybridization (ISH)

ISH is performed as described (Appleton, C T et al. (2006); J. Cell Physiology 207(3):735-745)). Probes for endogenous (mouse) and transgene (human) F-spondin is amplified by PCR, cloned into the pGEM T-Easy® vector (Promega) and sequenced. Vectors are linearized, and DIG-labeled sense and antisense riboprobes are produced with SP6 and T7 RNA polymerases using 10× DIG Labeling Mix (Roche), along with probes for known cartilage markers. Paraffin sections of bones and joints are dewaxed, digested with proteinase K, fixed and hybridized overnight at 55° C. with DIG-labeled sense or antisense riboprobes. Riboprobes are digested with RNase A and incubated with Anti-DIG primary antibody, conjugated to alkaline phosphatase (Roche). The blocking and detection of the DIG-labeled sections using NBT-BCIP colorimetric reaction is carried out according to the instructions of the manufacturer (Roche).

### 9A. Expression of F-Spondin in Cartilage In Vivo

F-spondin expression is examined during endochondral bone development and following medial meniscectomy of skeletally mature animals. Expression is studied in long bone and rib tissue sections from various embryonic and postnatal stages by in situ hybridization and immunohistochemistry (ISH/IHC). Similar studies are performed in the mouse OA model, from pre-surgery to 12 weeks post-surgery. Sham-operated mice are used as control. Surgery is performed on young mice (2-4 months) to avoid potential complications with superimposition of spontaneous OA, which can be apparent after 6 months of age. In situ analyses for localization of gene expression are complemented by quantitative analyses of cartilage-extracted RNA and protein by real-time PCR and western blotting/ELISA as described (Wang et al, submitted; Wu et al (2007) Arthritis Rheum 2007 November 56(11):3675-84). Parallel studies are performed in our rat model of OA to complement our existing microarray data.

### 9B. Overexpression of F-Spondin in the Cartilage of Transgenic Mice

Our expression data suggest a potential role of F-spondin in the pathogenesis of OA. To examine this possibility, a gain-of-function model is generated by overexpressing human F-spondin in transgenic mice under the control of the cartilage-specific collagen II promoter (Lefebvre V, Garofalo S, Zhou G, Metsaranta M, Vuorio E, De Crombrugghe B. Characterization of primary cultures of chondrocytes from type II collagen/beta-galactosidase transgenic mice. Matrix Biol 1994; 14(4):329-35). Mice are generated using standard procedures. Initially, 3-4 independent transgenic lines are examined to account for possible integration site or copy number effects. Transgenic lines are maintained on a C57BL/6J background and genotyped as described (Beier F, Leask T A, Hague S, Chow C, Taylor A C, Lee R J, et al. Cell cycle genes in chondrocyte proliferation and differentiation. Matrix Biol 1999; 18(2):109-20; Beier F, Ali Z, Mok D, Taylor A C, Leask T, Albanese C, et al. TGFbeta and PTHrP control chondrocyte proliferation by activating cyclin D1 expression. Mol Biol Cell 2001; 12(12):3852-63); Wang et al, submitted; Wu et al (2007) Arthritis Rheum 2007 November 56(11):3675-84). All transgenic mice are directly compared to wild type littermates. Transgene expression is examined in tissue sections (ISH/IHC) and quantified using real-time PCR and western blotting. In the first step, the effects of F-spondin overexpression on growth plate development and endochondral ossification is examines. Long bones from several developmental stages are examined for expression of cartilage marker genes, vascular invasion and ossification (ISH/IHC), proliferation (BrdU incorporation), apoptosis (TUNEL) and tissue organization, as described (Wang et al, submitted; Wu et al (2007) Arthritis Rheum 2007 November 56(11):3675-84). Alterations in key developmental pathways (Sox9, Runx2, IHH, PTH, FGFs, BMPs, VEGF etc) are examined by ISH/IHC. In addition, whether F-spondin overexpression affects TGF-β signaling is determined using IHC with phospho-specific Smad2/3 antibodies. Similar techniques are utilized to examine joint architecture in older mice (3-18 months) to examine whether F-spondin overexpression results in spontaneous cartilage degeneration. In parallel to the histological analyses, live animal microCT is performed to examine changes in subchondral bone and in joint space (Appleton et al, in prep.; McErlain et al Osteo Cartilage 2007 Sep. 25 Epub). Our medial meniscectomy model is employed to examine whether F-spondin overexpression alters the time course of joint degeneration. The studies described here are complemented by molecular and quantitative analyses (e.g., expression of OA markers in cartilage by real-time PCR, measurement of cytokines, proteases and ECM degradation products in urine and blood by ELISA etc.).

TABLE 3
Listing of Other Relevant GenBank Accession
Numbers and Sequence Identifiers

| | Gen Bank | | GenBank | |
|---|---|---|---|---|
| | Accession | SEQ | Accession | SEQ |
| Homo Sapiens | Number | ID | Number | ID |
| Gene Name | Nucleotide | No: | Protein | No: |
| SOX-9 | NM_000346 | 7 | NP_000337 | 8 |
| RUNX-2 | NM_001024630 | 9 | NP_001019801 | 10 |
| COX-2 (PGE2) | NM_000963 | 11 | NP_000954 | 12 |
| TGF-b1 | NM_000660 | 13 | NP_000651 | 14 |
| COL2A1 | NM_001844 | 15 | NP_001835 | 16 |
| Aggrecan | NM_001135 | 17 | NP_001126 | 18 |
| MMP-13 | NM_002427 | 19 | NP_002418 | 20 |
| MMP-1 | NM_002421 | 21 | NP_002412 | 22 |
| BMP-2 | NM_001200 | 23 | NP_001191 | 24 |
| IL-8 | NM_000584 | 25 | NP_000575 | 26 |
| IL-6 | NM_000600 | 27 | NP_000591 | 28 |
| IL-1beta | NM_000576 | 29 | NP_000567 | 30 |
| TNFalpha | NM_000594 | 31 | NP_000585 | 32 |
| Artificial sequence: | | 33 | | |
| primer | | | | |
| Fragment of human | | | | 34 |
| F-spondin: | | | | |
| TSR1: corresponds | | | | |
| to residues | | | | |
| 446-494 of | | | | |
| SEQ ID NO: 2 | | | | |
| Fragment of human | | | | 35 |
| F-spondin: | | | | |
| TSR2: corresponds | | | | |
| to residues | | | | |
| 504-552 of | | | | |
| SEQ ID NO: 2 | | | | |
| Fragment of human | | | | 36 |
| F-spondin: | | | | |
| TSR3: corresponds | | | | |
| to residues | | | | |
| 561-611 of | | | | |
| SEQ ID NO: 2 | | | | |
| Fragment of human | | | | 37 |
| F-spondin: | | | | |
| TSR4: corresponds | | | | |
| to residues | | | | |
| 618-666 of | | | | |
| SEQ ID NO: 2 | | | | |
| Fragment of human | | | | 38 |
| F-spondin: | | | | |
| TSR5: corresponds | | | | |
| to residues | | | | |
| 672-720 of | | | | |
| SEQ ID NO: 2 | | | | |
| Fragment of human | | | | 39 |
| F-spondin: | | | | |
| TSR6: corresponds | | | | |
| to residues | | | | |
| 759-807 of | | | | |
| SEQ ID NO: 2 | | | | |
| Fragment of human | | | | 40 |
| F-spondin: | | | | |
| F-spondin | | | | |
| signal sequence: | | | | |
| correspondes to | | | | |
| residues: 1-23 of | | | | |
| SEQ ID NO: 2 | | | | |

### Example 10

### F-Spondin Regulates Chondrocyte Maturation and Endochondral Bone Formation

This study reports a novel role for the thrombospondin-related, extracellular matrix protein, F-spondin, in embryonic cartilage. In developing chick tibia, F-spondin expression was found to localize to the hypertrophic and calcified zones of maturing cartilage. Functional studies using tibial organ cultures indicated that F-spondin inhibited longitudinal limb growth about 35% (p=0.02) relative to untreated controls. This was accompanied by increased alizarin red staining and greater numbers of hypertrophic chondrocytes, visualized by H&E staining. To investigate whether F-spondin regulates terminal maturation of chondrocytes, in vitro, we used a chick sternal model, in which differentiation is induced by retinoic acid (RA) treatment. We observed a 3.5-fold upregulation of F-spondin following RA treatment (p<0.05). F-spondin overexpression by plasmid transfection in these cells caused increased mineral deposition and an upregulation of AP and MMP-13 mRNA expression (p<0.05). All effects were dependent upon costimulation with RA. Using AP as a differentiation marker we then investigated the mechanism of F-spondin promaturation effects. F-spondin-mediated stimulation of AP activity following transient transfection was dependent on the presence of its thrombospondin (TSR) domain. Similarly coculture with a TSR domain specific F-spondin antibody inhibited RA induced AP activity 40% compared to controls (p<0.05). F-spondin mediated induction of AP gene expression was also found to be dependent upon TGF-β. Depletion of TGF-β from culture supernatants of F-spondin transfected cultures prevented its stimulatory effect on AP gene expression. Our findings indicate that F-spondin is expressed in embryonic cartilage, where it has the capacity to accelerate chondrocyte terminal differentiation via interactions in its TSR domain and TGF-β dependent pathways. Also, F-spondin plays a role in endochondral bone formation and growth.

### Introduction

The cartilage extracellular matrix (ECM) is essential for maintenance of chondrocyte phenotype and function. Under homeostatic conditions, a balanced equilibrium of synthesis and degradation or replacement of ECM components enables normal function of cartilage. Disruption of this equilibrium results in either osteoarthritis, a degenerative disease of permanent cartilage of the articular surfaces, or chondrodysplasias and dwarfism, diseases of the transient growth cartilage of the long bones.

While these two cartilages have the same embryonic origin they follow divergent differentiation pathways and fulfill different functions. In the articular, tracheal, and other permanent cartilage structures the chondrocytes maintain a stable phenotype and persist throughout life. In contrast, most of the embryonic cartilaginous skeleton, the epiphyseal growth plates of long bones, the cartilaginous callus formed at fracture sites, and the tissue created during distraction osteogenesis consist of transient cartilage^{1,2}. Through a series of maturational changes resulting in chondrocyte hypertrophy and ECM mineralization, this transient cartilage is gradually replaced by bone. These changes include an increase in alkaline phosphatase activity (O'Keefe, R. J., et al. (1990) Connective Tiss Res 24(1):53-66), elevated synthesis of type X collagen (Schmid, T. M., and Linsenmayer, T. F. (1985) J Cell Biol 100(2):598-605), 1985; Hoyland et al., 1991), and MMP-13 and, down-regulation of type II collagen production (Hillarby, M. C., et al. (1996) Ann NY Acad Sci 785:263-6) as well as, and raised secretion of bone matrix proteins including osteonectin (Metsaranta, M., et al. (1989) Calcif Tissue Int 45(3):146-52) and osteocalcin (Mark, M. P., et al. (1988) Differentiation 37(2): 123-36). The hypertrophic chondrocytes release matrix vesicles that serve as sites of nucleation for mineral formation (Anderson, H. C., (1969) J Cell Biol 41(1) 59-72) while avascular invasion of this calcified cartilage brings osteoprogenitor cells that resorb the cartilage and replace it with bone.

Articular chondrocytes under normal physiological conditions do not progress through these maturational stages. However, during OA, many of these cellular processes that characterize endochondral ossification during long bone development are recapitulated (Aigner, T., and Gerwin, N. (2007) Curr Drug Targets 8:377-85) Strikingly, during OA articular chondrocytes synthesize type X collagen (Walker, G. D., et al. (1995) J Orthop Res 13:4-12) (Von der Mark, K., et al. (1992) Arthritis Rheum 35:806-811) MMP-13 (Mitchell, P. G., et al. (1996) J Clin Invest 97(3): 761-8), osteopontin (Pullig, O., et al. (2000) Matrix Biol 19:245-55) and osteocalcin (Pullig, O., et al. (2000) Calcif Tissue Int 67:230-40), which are markers of hypertrophic chondrocytes. In addition, osteoarthritic chondrocytes exhibit high alkaline phosphatase activity, mineralize the extracellular matrix (Hashimoto, S., et al. (1998) Proc. Natl. Acad. Sci. USA 95: 3094-99) and die by apoptosis (Blanco, F. J, et al., (1995) Am JPathol. 146:75-85). Evidence from all these studies strongly supports the hypothesis that during osteoarthritis the articular chondrocyte switches its developmental program from a permanent cartilage cell to a transient cartilage one. Therefore, pathways controlling chondrocyte hypertrophy are extremely important not only as targets for growth therapies but also as targets for new therapies to OA. In this regard, developmental models of chondrocyte maturation and endochondral ossification are valuable tools for studying the function of OA-associated proteins.

We have previously reported upregulation of f-spondin in OA cartilage (see above examples; Attur, M et al (2009) FasebJ 23:79-89). Articular chondrocytes under normal physiological conditions do not progress through these maturational stages. However, during OA, many of these cellular processes that characterize endochondral ossification during long bone development are recapitulated (Aigner T, 2002) Strikingly, during OA articular chondrocytes synthesize type X collagen,³⁻⁵ MMP-13, osteopontin⁶ and osteocalcin⁷, markers of hypertrophic chondrocytes. In addition, osteoarthritic chondrocytes exhibit high alkaline phosphatase activity⁶ mineralize the extracellular matrix^{3,4,8,9}, and die by apoptosis^{10,11}. Evidence from all these studies strongly supports the hypothesis that during osteoarthritis the articular chondrocyte switches its developmental program from a permanent cartilage cell to a transient cartilage one. Therefore, pathways controlling chondrocyte hypertrophy are extremely important not only as targets for growth therapies but also as targets for new therapies to OA. In this regard, developmental models of chondrocyte maturation and endochondral ossification are valuable tools for studying the function of OA-associated proteins.

We have previously reported upregulation of f-spondin in OA cartilage (see above examples; Attur, M et al (2009) FasebJ 23:79-89). F-spondin is an ECM, heparin-binding glycoprotein that regulates neuronal outgrowth in the embryonic floor plate.¹². It is a member of a family of proteins that collectively belong to a subgroup of TSR (thrombospondin) type I class molecules, which include COMP, CTGF, ADAMTS-7&12 and CILP (Tucker, R. P. (2004) Int J Biochem Cell Biol 36:969-74).^{13,14}. In OA cartilage explant cultures, F-spondin treatment was found to increase both type II collagen degradation and MMP-13 secretion, suggesting it ability to regulate articular chondrocyte metabolism. However, F-spondin's role in normal cartilage biology, endochondral ossification and bone growth has not been described. In the present study, we examined F-spondin expression in growth plate cartilage and investigate its role in chondrocyte hypertrophy, using well established developmental biology models.

### Materials and Methods

### Immunohistochemistry of Embryonic Chick Tibia.

To determine whether F-spondin is expressed in embryonic cartilage, immunohistochemical analysis of sections of 18 day old chick tibial growth plates was performed. Specimens were fixed in 10% phosphate buffered formalin and decalcified in 4.1% disodium ETDA at 4° C. for 2 weeks. Following paraffin embedding and tissue processing, 5-μm-thick sections were cut, deparaffinized, rehydrated and immunostaining performed using a TSR domain specific, polyclonal F-Spondin antibody (R4; 1:100 diln) (the R4 antibody was kindly provided by Dr. Avihu Klar (Hebrew University, Jerusalem, Israel) and the Vectastain ABC kit (Vector Laboratories, Burlingame, Calif.) according to the manufacturer's instructions. Sections were counter stained with 1% alcian blue. The stained sections were mounted under glass coverslips, and scanned on Scan Scope GL series optical microscope (Aperio, Bristol, UK) at 20× magnification.

### Mouse Tibia Organ Culture.

All animal experiments were conducted according to the guidelines of the Institutional Animal Care and Use Committee (IACUC) of New York University. CD1 timed-pregnant mice (Charles River Laboratories, Wilmington, Mass.) were euthanized, and tibiae isolated from E15.5 embryos using a stereomicroscope (Nikon Instruments, Melville, N.Y.). Dissection day was considered day 0, and tibiae were allowed to recover from dissection overnight in serum-free D-MEM media containing 0.2% bovine serum albumin (BSA), 0.5 mM L-glutamine, 40 U/mL penicillin, and 40 μg/ml streptomycin as described by Serra and coworkers (1999). After 12 hours, tibiae were placed in 24-well Falcon plates and initial longitudinal length was measured using a stereomicroscope. Tibiae then were treated with either 0.5 μg/ml F-spondin recombinant protein (R&D Systems, Minneapolis, Minn.) or 1 μg/ml of F-spondin neutralizing antibody (R1). (purchased from R&D Systems, Minneapolis, Minn. (Cat No AF3135). Media was changed every 24 hours beginning on day one, and tibial length was measured again at the end of one week. The results were expressed as percentage changes in length relative to day one.

### Histological Staining of Mouse Tibiae.

For alizarin red/alcian blue staining (staining for mineral and proteoglycan respectively), at the end of the culture period tibiae were fixed with glycerol/ethanol [fixed with 1% glycine in 70% ethanol] and placed in staining solution (0.05% Alizarin red, 0.015% Alcian blue, 5% acetic acid in 70% ethanol) for 45-60 minutes. Digital images of stained tibia were collected using a stereomicroscope (Nikon Instruments, Melville, N.Y.) and a digital camera. To determine cellular organization after F-spondin treatment, tibiae were fixed, paraffin embedded, sectioned as described above and stained with Hematoxylin & Eosin.

### In Vitro Chondrocyte Maturation Model.

Chondrocytes were isolated from the cephalic (upper) portion of 14 day old chick embryo sterna as previously described (Teixeira, C. C., et al. (1995) Calcif Tissue Int 56:252-56).¹⁵. After 5 days of primary culture, floating chondrocytes were separated from the attached fibroblasts, and plated in tissue culture dishes in DMEM supplemented with 10% FBS and hyaluronidase (4-units/ml) to promote attachment. At confluence, differentiation was induced by daily treatment with all-trans retinoic acid (RA) from 0.1% v/v aliquots of a 1000-fold stock solution in 95% ethanol. Final concentrations ranged from 10 to 100 nM. To examine the effect of F-spondin overexpression, cultures were transfected at 70-80% confluence, with either full length (pFSI), truncated (pFS6) F-spondin cDNAs or control vector pcDNA3 (Invitrogen) using GenePorter transfection reagent (Genlantis, San Diego, Calif.). Differentiation was induced by daily treatments with 100 nM RA, which were initiated the following day. For F-spondin inhibition studies, a TSR domain specific F-spondin antibody (R1), or a spondin domain specific F-spondin antibody (R4) (R1 and R4 antibodies were provided by Dr. Avihu Klar (Hebrew University, Jerusalem, Israel), were added to chondrocyte cultures (1:100 dilution), every other day 1 h prior to RA treatment.

To study the effect of TGF-β depletion, chondrocytes were transfected with pcDNA3 or pFS1 and supernatants harvested after 48 h. Conditioned supernatants were then incubated with 5 mg/ml anti-TGF-β antibody (R&D Systems, Minneapolis, Minn.) for 30 mins at room temperature and added to freshly seeded chondrocytes in the presence of RA. After 24 h incubation, cells were harvested for determination of mRNA levels by RT-qPCR.

### Alkaline Phosphatase Activity Determination and Von Kossa Staining.

To measure levels of activity of alkaline phosphatase, cells were harvested in 0.1% triton-X 100 and 1/10^{th} volume was mixed with a freshly prepared solution of 1.5M tris-HCl pH 9.0 containing 7.5 mM p-nitrophenylphosphate, 1 mM ZnCl₂, and 1 mM MgCl₂. Changes in absorbance were measured spectrophotometrically at 410 nm for 10 min; changes over time correspond to the p-nitrophenylphosphate hydrolysis to p-nitrophenol. AP activity was expressed as nmol of product/min/mg of protein; 1 absorbance unit change corresponds to 64 nmol of product.¹⁶ To assess mineral deposition in F-spondin transfected cultures, cells were washed and incubated with 5% silver nitrate for 60 min under light, followed by incubation with 5% sodium thiosulfate for 2 min. Mineral accumulation was visualized by the appearance of a silver/grey precipitate on the cell surface.

### Gene Expression Analyses.

Different regions of the chick growth plate were visually identified and dissected into separate tubes, frozen in liquid nitrogen and crushed. Total RNA was extracted using Trizol® reagent (Life Technologies, Gaitherburg, Md.) per manufacturer's instructions. RNA was purified using RNA micro kit (Qiagen Inc, Chatsworth, Calif.) according to RNeasy cleanup protocol. Total RNA was also extracted from chondrocyte cultures and purified using Qiagen RNeasy mini columns according to the manufacturer's protocol (Qiagen Inc, Chatsworth, Calif.). One-step RT-PCR was performed using 20 ng total RNA and QuantiTect SYBR Green RT-PCR reagents, a DNA Engine Optican2 system (Roche Molecular Systems, Pleasanton, Calif.), and primers specific for chick genes: type X collagen (forward: AGTGCTGTCATTGATCTCATTGGA (SEQ ID NO: 41); reverse: TCAGAGGAATAGAGACCATTGGATT (SEQ ID NO: 42), type I collagen (forward: GCCGTGACCTCAGACTTAGC (SEQ ID NO: 43), reverse: TTTTGTCCTTGGGGTTCTTG (SEQ ID NO: 44), Collagen type II a1 (II) chainMMP-13. Acidic ribosomal protein (RP) mRNA was used as a reference for quantification as a housekeeping gene (forward: AACATGTTGAACATCTCCCC (SEQ ID NO: 45), reverse: ATCTGCAGACAGACGCTGGC (SEQ ID NO: 46)). Primers were purchased from Qiagen Inc (Valencia, Calif.). Relative expression levels of various transcripts were calculated using the 2-delta computed tomography method (Livak, K. J., and Schmittgen, T. D. (2001) Methods 25, 402-08). Statistical analyses.

Statistical analyses were performed using SPSS 13.0 (Chicago, Ill.). All experiments were repeated 3-4 times and the mean and standard deviation were determined. Significant differences between test groups and controls were assessed by ANOVA or student's t test. Significance was set at p<0.05.

### Results

Localized Expression of F-Spondin in Embryonic Growth Plate Cartilage.

Our previous work has identified F-spondin as a marker of osteoarthritic cartilage (Attur, M et al (2009) FasebJ 23:79-89). In this study, we investigated whether F-spondin is also expressed in differentiating embryonic chondrocytes during endochondral bone development by immunohistochemistry. We observed F-spondin expression within the growth plate cartilage of chick embryonic tibia (FIG. 19A). Strikingly, the brown positive staining was limited to the hypertrophic and mineralized regions of the growth cartilage. To confirm expression of F-spondin as a marker of chondrocyte maturation, we isolated the proliferative, hypertrophic and mineralized regions of tibial cartilage by micro-dissection and performed RT-PCR on mRNA extracted from the different regions. FIG. 19B shows the relative expression levels of F-spondin within each region, in parallel with established hypertrophic markers. As expected, type II collagen expression peaked in the proliferative region, while type X collagen was highest in the hypertrophic region. F-spondin expression, along with MMP 13, was highest within the calcified region, providing further evidence that it is a late stage marker of chondrocyte terminal differentiation.

F-Spondin Regulates Tibia Growth Ex Vivo.

To investigate the possible functional effects of F-spondin on endochondral bone formation and growth, we employed an organ culture system in which embryonic mouse tibia are isolated and cultured ex vivo in the presence of various modulators. Mouse tibia were cultured for 8 days with or without recombinant F-spondin (1 μg/ml) or a TSR-domain-specific F-spondin antibody. Tibia length was recorded daily and after 7 days specimens were prepared for alizarin red/alcian blue staining and histological analysis. Relative to untreated controls, F-spondin inhibited tibial growth approximately 37% (p=0.02; FIG. 20B). This was accompanied by a slight increase in alizarin red staining within the epiphysis of the tibia (FIG. 20B). In accordance with these observations, blocking F-spondin via antibody treatment led to the opposite effects: Limb growth was increased approximately 32% (p=0.008; FIG. 20B), and there was a modest reduction in alizarin red staining compared to control (untreated limbs) (FIG. 20A).

Chondrocyte morphology within the different regions of the growth cartilage was examined histologically at the end of the culture period (FIG. 20C). H&E stained sections show increased numbers of hypertrophic chondrocytes in the growth plate cartilage adjacent to the mineralized core, following F-spondin treatment. Conversely, inhibition of F-spondin by antibody treatment caused a reduction in the number of hypertrophic chondrocytes within the same region. Based on these observations we hypothesized that F-spondin functions to regulate limb growth by enhancing chondrocyte maturation within the growth plate.

F-Spondin is Expressed During RA Induced Maturation of Chick Sternal Chondrocytes in Vitro.

To investigate the effects of F-spondin in chondrocyte maturation, we used a well established in vitro model in which chick sternal chondrocytes mimic growth plate chondrocyte maturation in response to RA treatment. Increasing doses of RA (0-100 nm) for 5 days led to increased chondrocyte hypertrophy, consistent with previous observations (Iwamoto, M., et al. (1994) Microsc Res Tech 28(6): 483-91). M). FIG. 21A shows a dose dependant increase in AP activity (red staining) in response to RA. We next studied the expression of chondrocyte maturation markers in these cells by RT-PCR. Results show a marked downregulation of type II collagen, accompanied by a pronounced upregulation of type I collagen in response to RA treatment (FIG. 21B). Also corroborating our observations in the growth plate, late-stage maturation markers such as MMP-13 and VEGF increased with increased dose of RA. Interestingly, F-spondin also increased in a dose dependent manner, and was approximately 3-fold higher than controls in the presence of 100 nM RA (p=0.04; FIG. 21B). Somewhat surprisingly, type X collagen did not increase in response to RA. This may be reflected by the fact that, following isolation, cells were expressing high levels of type X collagen and thus already at the hypertrophic stage prior to RA treatment (data not shown). Therefore subsequent RA stimulation would likely ‘push’ the cells toward terminal maturation, resulting in a downregulation of type X collagen. Supporting this hypothesis, we found that the calcified region of the growth plate, containing terminally differentiated cells, had lower expression of type X collagen compared to the hypertrophic region (FIG. 19B).

F-Spondin Enhances Expression of Chondrocyte Maturation Markers.

We next examined the effects of F-spondin on maturation of chick sternal chondrocytes in vitro. Firstly, mineralization was assessed in RA-treated chondrocytes following overexpression of full length F-spondin cDNA (FS1) or control vector cDNA by plasmid transfection. F-spondin overexpression caused a moderate increase in mineral accumulation, detectable by von kossa staining, relative to vector controls (FIG. 22A). The same culture system was employed to assess the effects of F-spondin on the expression of chondrocyte maturation markers. In the presence of RA, F-spondin induced a 5-fold increase in MMP-13 and a 14-fold increase in AP expression, relative to pcDNA3-transfected controls (FIG. 22B). F-spondin had no effect on type X collagen and runx-2. Interestingly, in the absence of RA, F-spondin alone did not change the expression of these maturation markers (data not shown). These results indicate that in the presence of RA, F-spondin enhances chondrocyte terminal differentiation by increasing AP and MMP-13 expression, and enhancing mineralization.

F-Spondin Induces AP Activity Via its TSR Domain

We have previously shown that F-spondin effects on OA chondrocyte metabolism are mediated in part via its c-terminal thrombospondin-like TSR domain (Attur, M et al (2009) Faseb J 23:79-89). Using alkaline phosphatase activity as a quantitative measure of chondrocyte maturation, we investigated the role of the F-spondin TSR domain inhibition by either transient transfection of cDNA constructs or coculture with domain specific antibodies.

Overexpression of full-length F-spondin cDNA (FS1) increased AP activity approximately 3-fold compared to vector controls (FIG. 23B). In the absence of RA, F-spondin had no effect. This stimulatory effect was absent following overexpression of an F-spondin cDNA construct containing a deletion of the TSR domain (FS6) (FIG. 23A, B). Blocking endogenous F-spondin activity, via coculture with domain specific antibodies also resulted in an inhibition AP activity (FIG. 23C). The level of inhibition was dependent upon RA dose and antibody specificity. At higher doses of RA (35 and 100 nm), blocking the TSR domain caused a ˜40-50% inhibition of AP activity (p<0.02 vs ctrl; FIG. 23C), while targeting the n-terminal spondin domain had no inhibitory effect (FIG. 23C). These findings suggest that F-spondin induction of AP during chondrocyte maturation is mediated via its TSR domain.

We have previously reported that F-spondin can regulate the synthetic activity of articular chondrocytes via activation of latent TGF-β (Attur, M et al (2009) Faseb J 23:79-89). Since the TSR domain harbors conserved TGF-β binding motifs, we proposed that F-spondin-mediated enhancement of chondrocyte maturation similarly occurs via TGF-β. Consistent with this hypothesis, we observed that treatment of embryonic chick chondrocytes with TGF-β for 5 days led to dose dependent increases in expression of hypertrophic markers AP and MMP-13, as well as F-spondin (FIG. 24A). This finding indicates that TGF-β can act as an inducer of chondrocyte maturation, and may provide a mechanism for the promaturation effects of F-spondin. To determine whether TGF-II is required for F-spondin induction of AP, we transfected chick chondrocytes with FS1 or control vector cDNA and harvested conditioned media supernatants after 48 h. Supernatants were then depleted of TGF-β by coincubation with anti-TGF-b antibody and added to separate cultures of chick chondrocytes in the presence of retinoic acid for 24 h. Chondrocytes treated with conditioned media generated from FS1 transfected cultures demonstrated a 2-fold increase in AP gene expression compared to those receiving media from vector control transduced cells (open bars; FIG. 24B). Immunodepletion of TGF-β in the conditioned media of both control and FS1 transduced cultures led to a marked reduction in AP expression (grey bars; FIG. 24B). Since TGF-b depletion of the conditioned media of FS1 transfected cultures completely abolished its stimulatory effect on AP gene expression, our results suggest that TGF-β is required for F-spondin mediated induction of AP.

### DISCUSSION

In the present study we employed developmental models of chondrogenesis to investigate the role of F-spondin on chondrocyte phenotype and function. Our findings show that F-spondin is a late-stage marker of chondrocyte maturation. Functional studies indicate that F-spondin has the capacity to regulate endochondral bone growth in developing tibiae and promote hypertrophy and mineralization of cultured chondrocytes undergoing RA-induced maturation.

Gene expression profiling of mouse embryonic growth plate cartilage has also revealed F-spondin expression (Yamane, S., et al. (2007) Tissue Eng 13(9): 2163-73). F-spondin levels were approximately 20-fold higher in the resting zone of the growth plate compared to surface articular chondrocytes, however the authors did not compare expression between zones. In our study, positive staining for F-spondin was observed solely within the hypertrophic and calcified zones (FIG. 19A) of chick tibia, while mRNA expression gradually increased with terminal differentiation (FIG. 19B; FIG. 21B). Consistent with this observation, in developing mouse tibia (E15.5) we have observed the highest expression of F-spondin in the mineralized mid-diaphysis zone by microarray analysis (data not shown).

Our organ culture experiments indicate that F-spondin inhibits limb growth while increasing mineralization of growing tibiae. H&E staining of F-spondin treated limbs indicated increased numbers of hypertrophic chondrocytes adjacent to the bone forming dyaphysis. Accordingly, F-spondin inhibition, via antibody treatment had the reverse effect. We propose that inhibition of limb growth occurs due to accelerated chondrocyte hypertrophy and premature mineralization, at the expense of cell proliferation. F-spondin gene deletion studies are being performed to confirm this hypothesis. Interestingly, other TSR (thrombospondin) type I class molecule family members have been shown to play a role in normal skeletal development. Mutations in the cartilage oligomeric matrix protein (COMP) have been reported to cause pseudoachondroplasias, a well described form of dwarfism also associated with severe osteoarthritis requiring joint replacement (Hecht, J. T., et al. (1995) Nat Genet. 10(3):325-9; Briggs, M. D., et al. (1995) Nat Genet. 10(3): 330-6; Adams and Horton 1998). Murine models of pseudoachondroplasia, generated by engineering mutations in both globular and thrombospondin domains of COMP, exhibit short limb dwarfism, and growth plate disorganization (Schmitz, M., et al. (2008) Matrix Biol 27(2): 67-85; and Pirog-Garcia, K. A., et al. (2007) Hum Mol Genet. 16(17):2072-88), suggesting a deregulation of chondrocyte terminal differentiation.

While F-spondin function is most closely associated with neuronal outgrowth and differentiation, there has been more recent evidence it can also promote mineralization of connective tissues. Kitagawa et al. have reported upregulation of F-spondin in cementoblasts—specialized cells that deposit a calcified matrix of cementum at the roots of teeth (periodontium) (Kitagawa et al. (2006) Biochem Biophys Res Commun 349(3):1050-6). Overexpression of F-spondin in periodontal ligament cells, possible progenitors of cementoblasts, caused an upregulation of bone-related proteins including alkaline phosphatase and MMP-13. Interestingly, as in our model, F-spondin had no effect on gene expression of Runx-2, suggesting an alternate mechanism of osteoinduction.

To begin to elucidate the mechanism of F-spondin-mediated acceleration of chondrocyte maturation, we investigated the effects of blocking specific protein domains. We found TSR-dependent regulation of AP activity following stimulation with RA. In accordance with our observations, others have reported TSR-domain dependent biological effects of F-spondin, including floor plate cells migratory and growth promoting activities (Burstyn-Cohen T et al. 1999) Neuron 23(2): 233-46).[1, 2]. In OA chondrocytes, we have observed TSR-dependent induction of PGE2 (Attur, M et al (2009) Faseb J 23:79-89). This region also harbors a highly conserved latency associated peptide (LAP)-binding KFRK motif, which may be responsible for latent TGF-β activation. Indeed, addition of F-spondin to cartilage explant cultures was found to increase active TGFβ-1 levels in culture supernatants (Attur, M et al (2009) Faseb J 23:79-89). At present it is not clear if F-spondin effects on chondrocyte maturation are fully TGF-β dependant. While TGF-β is generally regarded as an inhibitor of chondrocyte hypertrophy, increased TGF-β activation has also been reported in chick embryonic chondrocytes undergoing terminal differentiation (D'angelo, M., et al. (2001) J Bone Miner Res 16(12):2339-47) Moreover, activation was found to be dependant on MMP-13.

In conclusion, the thrombospondin-related protein, F-spondin, is expressed in embryonic growth plate cartilage and can enhance the expression of chondrocyte maturation markers, providing a regulatory role for this protein in chondrocyte terminal differentiation, ECM mineralization and endochondral bone formation.

### REFERENCES

1. Iwamoto M, Higuchi Y, Enomoto-Iwamoto M, Kurisu K, Koyama E, Yeh H et al. The role of ERG (ets related gene) in cartilage development. Osteoarthritis Cartilage 2001; 9 Suppl A:S41-47.
2. Ferguson C, Alpern E, Miclau T, Helms J A. Does adult fracture repair recapitulate embryonic skeletal formation? Mech Dev 1999; 87:57-66.
3. Walker G D, Fischer M, Gannon J, Thompson R C, Jr., Oegema T R, Jr. Expression of type-X collagen in osteoarthritis. J. Orthop. Res. 1995; 13:4-12.
4. von der M K, Kirsch T, Nerlich A, Kuss A, Weseloh G, Gluckert K et al. Type X collagen synthesis in human osteoarthritic cartilage. Indication of chondrocyte hypertrophy. Arthritis Rheum. 1992; 35:806-811.
5. Eerola I, Salminen H, Lammi P, Lammi M, von der Mark K, Vuorio E et al. Type X collagen, a natural component of mouse articular cartilage: association with growth, aging, and osteoarthritis. Arthritis Rheum 1998; 41:1287-1295.
6. Pullig O, Weseloh G, Gauer S, Swoboda B. Osteopontin is expressed by adult human osteoarthritic chondrocytes: protein and mRNA analysis of normal and osteoarthritic cartilage. Matrix Biol 2000; 19:245-255.
7. Pullig O, Weseloh G, Ronneberger D, Kakonen S, Swoboda B. Chondrocyte differentiation in human osteoarthritis: expression of osteocalcin in normal and osteoarthritic cartilage and bone. Calcif Tissue Int 2000; 67:230-240.
8. Hashimoto S, Ochs R L, Rosen F, Quach J, McCabe G, Solan J et al. Chondrocyte-derived apoptotic bodies and calcification of articular cartilage. Proceedings of the National Academy of Sciences of the United States of America 1998; 95:3094-3099.
9. Hashimoto S, Creighton-Achermann L, Takahashi K, Amiel D, Coutts R D, Lotz M. Development and regulation of osteophyte formation during experimental osteoarthritis. Osteoarthritis Cartilage 2002; 10:180-187.
10. Blanco F J, Ochs R L, Schwarz H, Lotz M. Chondrocyte apoptosis induced by nitric oxide. American Journal of Pathology 1995; 146:75-85.
11. Aigner T, Kurz B, Fukui N, Sandell L. Roles of chondrocytes in the pathogenesis of osteoarthritis. Curr Opin Rheumatol 2002; 14:578-584.
12. Klar A, Baldassare M, Jessell T M. F-spondin: a gene expressed at high levels in the floor plate encodes a secreted protein that promotes neural cell adhesion and neurite extension. Cell 1992; 69:95-110.
13. Feinstein Y, Klar A. The neuronal class 2 TSR proteins F-spondin and Mindin: a small family with divergent biological activities. Int J Biochem Cell Biol 2004; 36:975-980.
14. Tucker R P. The thrombospondin type 1 repeat superfamily. Int J Biochem Cell Biol 2004; 36:969-974.
15. C. Teixeira C, Ischiropoulos H, Leboy P S, Adams S L, Shapiro I M. Nitric oxide mediates retinoic-acid induced chondrocyte maturation.; 2001.
16. Teixeira C C, Hatori M, Leboy P S, Pacifici M, Shapiro I M. A rapid and ultrasensitive method for measurement of DNA, calcium and protein content, and alkaline phosphatase activity of chondrocyte cultures. Calcified Tissue International 1995; 56:252-256.

1. A method for screening, diagnosing or prognosing a cartilage degenerative condition in a subject, or for measuring cartilage degeneration resulting from aging, trauma or a sports related injury in a subject, or for monitoring the state of chondrocyte cell transplant to a lesioned area, wherein said condition or said cartilage degeneration is characterized by an increase in the level of expression of F-spondin, said method comprising:
(I) measuring an amount of an F-spondin gene or gene product, or a fragment thereof, in a tissue sample obtained from the subject, wherein said F-spondin gene or gene product is:
(a) a DNA corresponding to any one of SEQ ID NOS: 1, 3 or 5 or a nucleic acid derived therefrom;
(b) a protein comprising any one of SEQ ID NOS: 2, 4 or 6;
(c) a nucleic acid comprising a sequence hybridizable to any one of SEQ ID NOS: 1,3 or 5, or their complements under conditions of high stringency, or a protein comprising a sequence encoded by said hybridizable sequence;
(d) a nucleic acid at least 90% homologous to any one of SEQ ID NOS: 1, 3 or 5, or their complements as determined using the NBLAST algorithm; or a protein encoded thereby; and
(II) comparing the amount of said F-spondin gene or gene product in said subject with the mount of F-spondin gene or gene product present in a normal tissue sample obtained from a subject who does not have a cartilage degenerative condition characterized by an increase in the level of expression of F-spondin, or in a predetermined standard, wherein an increase in the amount of said F-spondin gene or gene product in said subject compared to the amount in the normal tissue sample or pre-determined standard indicates the presence of a cartilage degenerative condition in said subject.

2. The method of claim 1, wherein said cartilage degenerative condition is selected from the group consisting of osteoarthritis, rheumatoid arthritis, psoriatic arthritis and chondrosarcomas.

3. The method of claim 1, wherein the measuring of said F-spondin gene or gene product is achieved by a method selected from the group consisting of reverse transcription-polymerase chain reaction (RT-PCR), real time PCR, northern blot analysis, in situ hybridization, cDNA microarray, electrophoretic gel analysis, an enzyme immunoassay (ELISA assay), immunohistochemistry, a Western blot, a dotblot analysis, a protein microarray, a flow cytometric technique, mass spectroscopy and proteomics analysis.

4. The method of claim 3, wherein the enzyme immunoassay is a competitive assay or a sandwich technique, and wherein antibody binding in combination with a reporter molecule is used to quantify the F-spondin gene product.

5. The method of claim 4, wherein the reporter molecule is selected from the group consisting of an enzyme, a fluorophore, a radiolabel, a colored dye, a light absorbing dye, a chemiluminescent molecule and a heavy metal.

6. The method of claim 5, wherein the heavy metal is colloidal gold.

7. The method of claim 3, wherein the proteomics analysis is accomplished by 2-dimensional polyacrylamide gel electrophoresis (2DE) coupled to mass spectrometry (MS).

8. The method of claim 1, wherein the tissue sample is selected from the group consisting of whole blood, blood cells, whole blood cell lysates, serum, plasma, urine, bone marrow, cerebrospinal fluid, saliva, chondrocytes, cartilage, synovium and synovial fluid.

9. The method of claim 8, wherein the blood cells are selected from the group consisting of white blood cells or red blood cells.

10. The method of claim 9, wherein the white blood cells are selected from the group consisting of lymphocytes, monocytes or macrophages, neutrophils, basophils and eosinophils.

11. The method of claim 1, wherein said method is used for monitoring the effect of therapy administered to a subject having a cartilage degenerative condition.

12. A method for screening, diagnosing or prognosing a bone condition, disease, disorder or degenerative condition in a subject, or for measuring bone degeneration or endochondral bone formation resulting from fracture, cancer, ageing, trauma or a sports related injury in a subject, wherein said condition, disorder or said degeneration is characterized by an alteration in the level of expression of F-spondin, said method comprising:
(I) measuring an amount of an F-spondin gene or gene product, or a fragment thereof, in a tissue sample obtained from the subject, wherein said F-spondin gene or gene product is:
(a) a DNA corresponding to any one of SEQ ID NOS: 1, 3 or 5 or a nucleic acid derived therefrom;
(b) a protein comprising any one of SEQ ID NOS: 2, 4 or 6;
(c) a nucleic acid comprising a sequence hybridizable to any one of SEQ ID NOS: 1,3 or 5, or their complements under conditions of high stringency, or a protein comprising a sequence encoded by said hybridizable sequence;
(d) a nucleic acid at least 90% homologous to any one of SEQ ID NOS: 1, 3 or 5, or their complements as determined using the NBLAST algorithm; or a protein encoded thereby; and
(II) comparing the amount of said F-spondin gene or gene product in said subject with the amount of F-spondin gene or gene product present in a normal tissue sample obtained from a subject who does not have a bone condition, disease, disorder or degenerative condition or in a predetermined standard, wherein an alteration in the amount of said F-spondin gene or gene product in said subject compared to the amount in the normal tissue sample or pre-determined standard indicates the presence of a bone condition, disease, disorder or degenerative condition in said subject.

13. The method of claim 12, wherein said bone condition, disease, disorder or degenerative condition is selected from the group consisting of bone fracture, bone cancer, brittle bone disease, osteoporosis, spondylosis, osteoporosis, fracture healing, skeletal dysplasias, osteochondrodysplasias and dwarfism.

14. A method for evaluating the effectiveness of therapy with an agent useful for treating a cartilage degenerative condition or a bone disease, disorder or condition, comprising collecting a series of tissue or cellular samples from a subject suffering from a cartilage degenerative condition or a bone disease, disorder or condition, wherein the samples are obtained before the initiation of therapy and during treatment with the agent and measuring the level of F-spondin, or a fragment thereof, in the subject using the method according to claim 12 wherein a normalization of F-spondin, or a fragment thereof, correlates with the effectiveness of therapy with the agent.

15. The method of claim 12, wherein the measuring of the F-spondin gene or gene product correlates with a change in the level of expression of at least one gene or gene product, which is a member of the PGE2, TGF-P or avβ3 pathways.

16. The method of claim 12, wherein the measuring of the F-spondin gene or gene product correlates with an increase in expression of at least one gene or gene product selected from the group consisting of COL2A, aggrecan, MMP-13, BMP2 and PGE2; or with a decrease in expression of at least one gene or gene product selected from the group consisting of MMP-1 and TNF-α; or with activation of latent TGF-βI.

17. A method of measuring chondrocyte hypertrophy in a sample, wherein said hypertrophy is the result of an increase in the level of expression of F-spondin, the method comprising hybridizing a probe derived from the nucleic acid of any one of SEQ ID NOS: 1,3 or 5, or a portion of at least 15-25 nucleotides thereof, or a full complement thereof, with a nucleic acid from said sample, wherein said hybridizing is indicative of chondrocyte hypertrophy resulting from an increase in the level of expression of F-spondin.

18. (canceled)

19. (canceled)

20. (canceled)

21. The method of claim 12, wherein the method further comprises evaluating the disease or condition using a method selected from the group consisting of ultrasound, MRI, CT scan, bone scan, X-ray analysis, or evaluation of synovial fluid aspirate.

22. A method of screening for an agent or a candidate compound that blocks or inhibits Fspondin expression or activity/function, the method comprising:
(a) contacting the F-spondin molecule, or fragments thereof, or cells containing the F-spondin molecule, with an agent or a candidate compound, wherein said F-spondin molecule comprises the nucleic acid sequence of any one of SEQ ID NOS: 1, 3 or 5 and/or the amino acid sequence of any one of SEQ ID NOS: 2, 4 or 6; and
(b) determining the level of F-spondin expression or activity/function in the presence or absence of the agent or candidate compound;
wherein the agent or candidate compound is considered to be effective if the level of F-spondin expression or activity/function is lower in the presence of the agent or candidate compound as compared to in the absence of the agent or candidate compound.

23. The method of claim 22, further comprising:
(c) measuring the effect of the candidate compound on the level of expression or activity/function of at least one gene or gene product, which is a member of the PGE2, TGF-β or αvβ3 pathways.

24. The method of claim 23, wherein the member of the PGE2, TGF-β or αvβ3 pathways is selected from the group consisting of COL2A, aggrecan, MMP-13, BMP2, PGE2, MMP-1, TNFα, and TGF-β1; wherein the candidate compound is identified as a positive candidate compound if the expression or activity of one or more molecules selected from the group consisting of COL2A, aggrecan, MMP-13, BMP2 and PGE2 is decreased in the presence, but not the absence of the candidate compound; or if the expression or activity of one or more molecules selected from the group consisting of MMP-land TNF-a is increased in the presence, but not the absence of the candidate compound; or if activation of latent TGF-α is inhibited in the presence, but not the absence of the candidate compound.

25. A method of screening for an agent or a candidate compound capable of modulating the expression or activity/function of F-spondin, said method comprising:
(a) contacting the F-spondin molecule, or a cell containing F-spondin, or a fragment thereof with said agent or candidate compound, wherein said F-spondin molecule is:
(i) a DNA corresponding to any one of SEQ ID NOS: 1, 3 or 5;
(ii) a protein comprising any one of SEQ ID NOS: 2, 4 or 6;
(iii) a nucleic acid comprising a sequence hybridizable to any one of SEQ ID NOS: 1, 3 or 5, or a complement thereof under conditions of high stringency, or a protein comprising a sequence encoded by said hybridizable sequence; or
(iv) a nucleic acid at least 90% homologous to any one of SEQ ID NOS: 1,3 or 5, or a complement thereof as determined using an NBLAST algorithm or a protein encoded thereby;
(b) determining whether or not the agent or candidate compound modulates the expression or activity/function of the F-spondin molecule;
wherein an agent or a candidate compound that increases the expression or activity/function of the F-spondin molecule, or a fragment thereof is considered to be an agonist of F-spondin, and wherein a candidate compound that decreases the expression or activity/function of the F-spondin molecule or a fragment thereof is considered to be an antagonist of F-spondin.

26. The method of claim 25, further comprising:
(c) measuring the effect of the agent or candidate compound on the level of expression or activity/function of at least one gene or gene product, which is a member of the PGE2, TGF-β or avβ3 pathways.

27. The method of claim 26, wherein the member of the PGE2, TGF-β or avβ3 pathways is selected from the group consisting of COL2A, aggrecan, MMP-13, BMP2, PGE2, MMP-1, TNFα, and TGF-β1;
wherein an agent or a candidate compound is identified as an agonist of F-spondin if the agent or candidate compound increases the expression or activity/function of one or more of the molecules selected from the group consisting of COL2A, aggrecan, MMP-13, BMP2 and PGE2;
and/or decreases the expression or activity/function of one or more of the molecules selected from the group consisting of MMP-I and TNF-a; and/or activates latent TGF-β1; and
wherein an agent or a candidate compound is identified as an antagonist of F-spondin if the candidate compound decreases the expression or activity/function of one or more of the molecules selected from the group consisting of COL2A, aggrecan, MMP-13, BMP2 and PGE2; and/or increases the expression or activity/function of one or more of the molecules selected from the group consisting of MMP-I and TNF-a; and/or prevents or inhibits activation of latent TGF-β.

28. (canceled)

29. (canceled)

30. (canceled)

31. (canceled)
